(19) United States
(12) Patent Application Publication
(10) Pub. No.: US 2003/0087243 A1
(43) Pub. Date: May 8, 2003

(54) Diagnosis, prognosis and treatment of cancer related to the Barx2 gene

(76) Inventors: Barry D. Nelkin, Baltimore, MD (US); Hani Gabra, Edinburgh (GB); Grant C. Sellar, Edinburgh (GB); Janet E. V. Watson, Edinburgh (GB); David John Porteous, Edinburgh (GB)

Correspondence Address: NIXON & VANDERHYE P.C., 8th Floor, 1100 North Glebe Road, Arlington, VA, 22201, US

(21) Appl. No.: 10/012,456

(22) Filed: Dec. 12, 2001

Related U.S. Application Data
(63) Continuation of application No. PCT/GB00/02328, filed on Jun. 15, 2000.
(60) Provisional application No. 60/139,320, filed on Jun. 15, 1999.

(30) Foreign Application Priority Data
Mar. 8, 2000 (GB) 0005466.8

(51) Int. Cl. C12Q 1/68

(52) U.S. Cl. 435/6

(57) ABSTRACT

5Surprisingly, it has been found that the Barx2 gene is mutated in ovarian cancer. The invention provides methods of diagnosis, prognosis and treatment of cancer related to the Barx2 gene.

### The present invention relates to cancer and in particular to ovarian cancer.

Cancer is a serious disease and a major killer. Although there have been advances in the diagnosis and treatment of certain cancers in recent years, there is still a need for improvements in diagnosis and treatment.

Cancer is a genetic disease and in most cases involves mutations in one or more genes. There are believed to be around 200,000 genes in the human genome but only a handful of these genes have been shown to be involved in cancer. Although it is surmised that many more genes than have been presently identified will be found to be involved in cancer, progress in this area has remained slow despite the availability of molecular analytical techniques. This may be due to the varied structure and function of genes which have been identified to date which suggests that cancer genes can take many forms and have many different functions.

Ovarian cancer is the most frequent cause of death from gynaecological malignancies in the Western World, with an incidence of 5,000 new cases every year in England and Wales. It is the fourth most common cause of cancer mortality in American women. The majority of patients with epithelial ovarian cancer present at an advanced stage of the disease. Consequently, the 5 year survival rate is only 30% after adequate surgery and chemotherapy despite the introduction of new drugs such as platinum and taxol (Advanced Ovarian Cancer Trialists Group (1991) BMJ 303, 884-893; Ozols (1995) Semin Oncol. 22, 61-66). However, patients who have stage I disease (confined to the ovaries) do better with the 5 year survival rate being 70%. It is therefore desirable to have techniques to detect the cancer before metastasis to have a significant impact on survival.

Epithelial ovarian cancer constitutes 70-80% of ovarian cancer and encompasses a broad spectrum of lesions, ranging from localized benign tumours and neoplasms of borderline malignant potential to invasive adenocarcinomas. Histologically, the common epithelial ovarian cancers, are classified into several types, that is, serous, mucinous, endometrioid, clear cell, Brenner, mixed epithelial, and undifferentiated tumours. The heterogeneity of histological subtypes reflects the metaplastic potential of the ovarian surface Mullerian epithelium which shares a common embryological origin with the peritoneum and the rest of the uro-genital system. Germ cell, sex cord/stromal tumours and sarcomas represent the remainder of ovarian cancers. The histogenesis and biological characteristics of epithelial ovarian cancer are poorly understood as are the molecular genetic alterations that may contribute to the development of such tumours or their progression. Epidemiological factors related to ovulation seem to be important, whereby ovarian epithelial cells undergo several rounds of division and proliferative growth to heal the wound in the epithelial surface. These lead to the development of epithelial inclusion cysts and frank malignant tumours may arise from them (Fathalla (1971) Lancet 2, 163).

A review of ovarian cancer screening is given in Bell et al (1998) Health Technology Assessment 2, 1-50.

Genetic changes in the tumour are critical for the development of cancer. Many chromosomal regions (chromosomes 3, 5, 6, 8, 11, 13, 17, 18, 22, and X) have been implicated to contain tumour suppressor genes involved in tumour progression of sporadic ovarian cancer, but only the p53 gene (chromosome arm 17p) has been found to be frequently mutated (Shelling et al (1995) Br. J. Cancer 72, 521-527). The BRCA1 gene (chromosome arm 17q) and the BRCA2 gene (chromosome arm 13q) isolated in 1994 and 1996 respectively, are mutated in a proportion of patients with familial breast/ovarian cancer (Ford & Easton (1995) Br. J. Cancer 72, 805-812). Familial ovarian cancer only accounts for 5-10% of all ovarian tumours. In tumours from patients with sporadic ovarian cancer, only five mutations in the BRCA1 gene and four in the BRCA2 gene have been reported (Takahashi et al (1995) Cancer Res. 55, 2998-3002; Takahashi et al (1996) Cancer Res. 56, 2738-2741) suggesting that they are rare in sporadic ovarian cancer. Mutations in the mismatch repair genes have been reported at a frequency of 10% (Tangi et al (1996) Cancer Res. 56, 2501-2505; Fujita et al (1995) Int. J. Cancer 64, 361-366; Orth et al (1994) Proc. Natl. Acad. Sci. USA 91, 9495-9499). Thus genes that may be more critical in tumour progression in sporadic ovarian cancer have not yet been fully characterised.

WO 96/05306, WO 96/05307 and WO 96/05308 relate to methods and materials used to isolate and detect a human breast and ovarian cancer predisposing gene (BRCA1), some mutant alleles of which are alleged to cause susceptibility to cancer, in particular breast and ovarian cancer.

Tumour suppressor activity has been suggested to be encoded on chromosome 11 (Tanaka et al (1991) Nature 349, 340-342; Rimessi et al (1994) Oncogene 9, 3467-3474; Satoh et al (1993) Mol. Carcinogenesis 7, 157-164; Yoshida et al (1994) Mol. Carcinogenesis 9, 114-121; Gabra et al (1996) Int. J. Oncol. 8, 625-631; Gabra et al (1996) Cancer Res. 56, 950-954; Gabra et al (1995) Br. J. Cancer 72, 367-375; EP 0 727 486; Gabra et al (1998) Proc. AACR 39, Abstract #4236; and Gabra et al (1998) Br J. Cancer 78, Poster P185) but none of these papers identify a candidate gene, nor do they provide any evidence for a single gene being involved in tumour suppressor activity.

Colorectal tumours of the large intestine are a frequent cause of human cancer mortality in the Western world with approximately 19,000 deaths in the UK per annum.

The majority of cancers of the colorectum are adenocarcinomas (Jass and Morson (1987) J. Clin. Pathol. 40, 1016-1023; Morson (1974) Proc. R. Soc. Med. 67, 451-457). The literature remains divided on the true origins of colorectal carcinomas and it has been proposed that carcinomas may arise both from within existing benign neoplasms (termed adenomas), in what has been termed the adenoma to carcinoma sequence (Muto et al (1975) Cancer 30, 2251-2270), or via areas of generalised dysplasia (de novo) without an adenomatous stage. Whilst it is probable that some colorectal cancers originate in adenomas, the majority of adenomas do not appear to progress to carcinoma and indeed may even regress (Knoernschild (1963) Surg. Forum XIV 137-138). Whilst evidence on environment, diet, age and sex suggest that these are all risk factors for colorectal cancer, the lack of confirmation of involvement of these factors in all cases suggests an underlying genetic basis for colorectal tumour formation. The majority of colorectal cancers are not associated with clear inherited syndromes although hereditary forms do exist, including Familial Polyposis Coli (FPC), Gardner's Syndrome, Hereditary non-Polyposis Colorectal Cancer (HNPCC) and Turcot's Syndrome.

Several oncogenes and tumour suppressor genes have now been shown to play a definite role in colorectal tumorigenesis, whilst at other loci a correlation between LOH and colorectal cancer is less well defined.

Jones et al (1997) Proc. Natl. Acad. Sci. USA 94, 2632-2637 describes the mouse Barx2 gene, a homeobox gene expressed in neural and craniofacial structures. The human homologue of the Barx2 gene has been described in Gen Bank accession number AF031924 where it is described as being a ras-responsive transcription factor and a candidate for involvement in Jacobsen syndrome which is a rare congenital disorder typically characterised by a number of craniofacial abnormalities, heart defects and thrombocytopeenia (Jacobsen et al (1973) Hum. Hered. 23, 568-585; Lewanda et al (1995) Am. J. Hum. Genet. 59, 193-198; and Penny et al (1995) Am. J. Hum. Genet. 56, 676-683). In the chick, the Barx2 gene appears to be a marker for myogenic cells also expressed in branchial arches and neural structures (Smith & Tabin (1999) Mechanisms of Development 80, 203-206).

Surprisingly, out of the plethora of genes in the genome, it has now been found that the Barx2 gene is mutated in sporadic epithelial ovarian cancer, and the 5′ end of the Barx2 transcript is not expressed in several ovarian cancer cell lines. Furthermore, we have shown that expression of Barx2 suppresses growth in certain ovarian cancer cell lines, and northern blot analysis indicates that several ovarian cancer cell lines do not express Barx2. It is believed that the Barx2 gene is involved in ovarian cancer as a tumour suppressor gene.

The protein encoded by the Barx2 gene binds DNA. A property of the Barx2 polypeptide is the ability to bind the consensus sequence YYTAATGRTTTTY.

A first aspect of the invention provides a method for determining the susceptibility of a patient to cancer comprising the steps of (i) obtaining a sample containing nucleic acid from the patient; and (ii) contacting the said nucleic acid with a nucleic acid which hybridises selectively to the Barx2 gene, or a mutant allele thereof, or a nucleic acid which hybridises selectively to Barx2 cDNA, or a mutant allele thereof, or their complement.

A second aspect of the invention provides a method of diagnosing cancer in a patient comprising the steps of (i) obtaining a sample containing nucleic acid from the patient; and (ii) contacting the said nucleic acid with a nucleic acid which hybridises selectively to the Barx2 gene, or a mutant allele thereof, or a nucleic acid which hybridises selectively to Barx2 cDNA, or a mutant allele thereof, or their complement.

A third aspect of the invention provides a method of predicting the relative prospects of a particular outcome of a cancer in a patient comprising the steps of (i) obtaining a sample containing nucleic acid from the patient; and (ii) contacting the said nucleic acid with a nucleic acid which hybridises selectively to the Barx2 gene, or a mutant allele thereof, or a nucleic acid which hybridises selectively to Barx2 cDNA, or a mutant allele thereof, or their complement. Identification of mutations in, or lack of activity of, Barx2 are believed to be particularly useful for prognosis (ie link to poorer outcome) and in determining whether a patient may be one suitable for treatment by gene therapy (see below).

Preferably, the patient is a human patient and, generally, reference to Barx2 is a reference to human Barx2.

The Barx2 gene is located on various PAC clones from library No. 709 (RPCI6) from the Resource Centre/Primary Database (RZPD) of the German Human Genome Project at the Max Planck Institute for Molecular Genetics, Heubrerweg 6, 14059 Berlin-Charlottenburg, Germany (www.rzpd.de). Exons 1, 2, 3 and 4 of the Barx2 gene are found on PAC1 from this library (PAC1 corresponds to picked clone LLNLP709O0720Q2 from this library; see Examples for further details). It will readily be appreciated by the skilled person that the Barx2 gene or parts thereof may readily be obtained from other suitable human gene libraries, such as standard cosmid, or yeast artificial chromosome (YAC) or P1-artificial chromosome (PAC) libraries using the aforementioned PAC clones, or fragments thereof, as probes. Similarly, a Barx2 cDNA may be used as a probe to identify all or parts of the Barx2 gene.

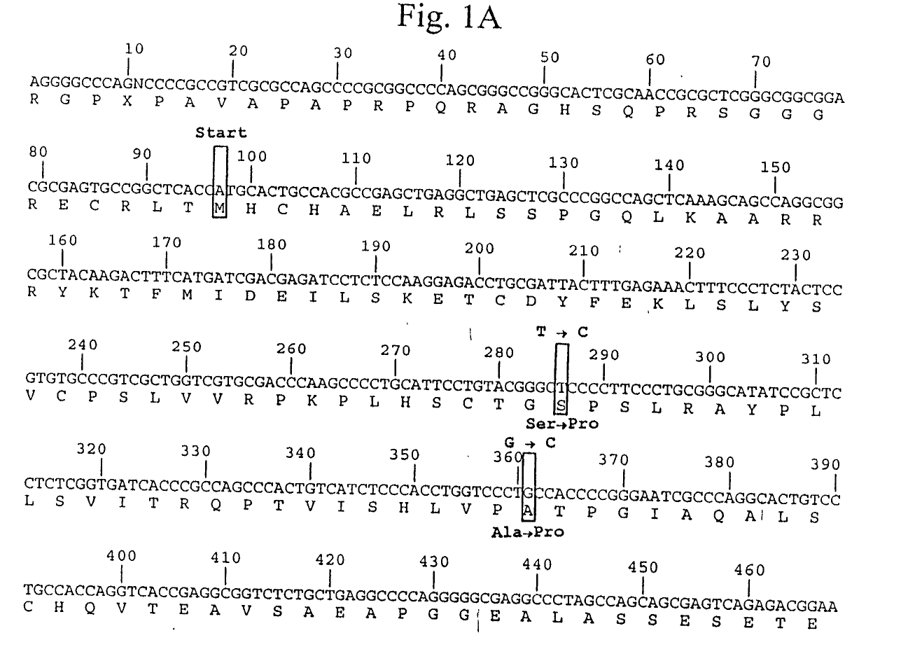

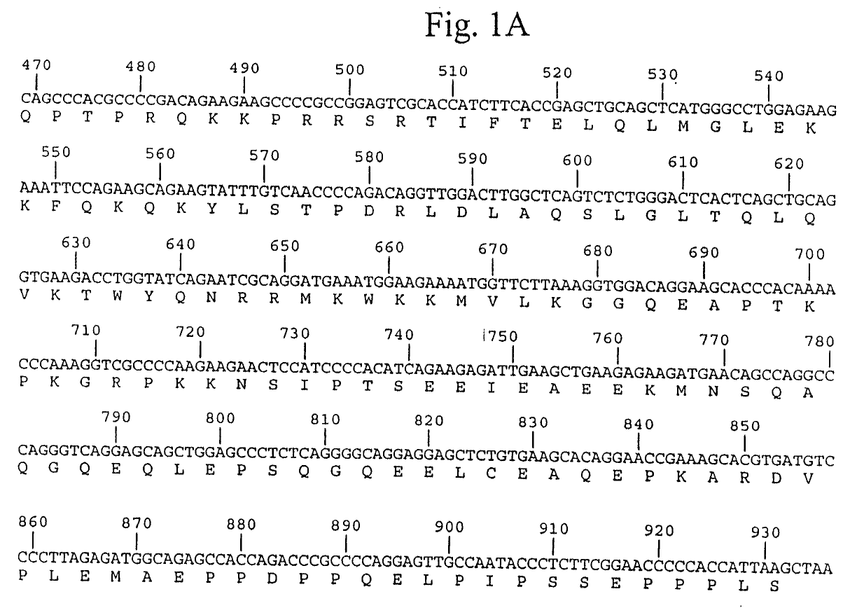

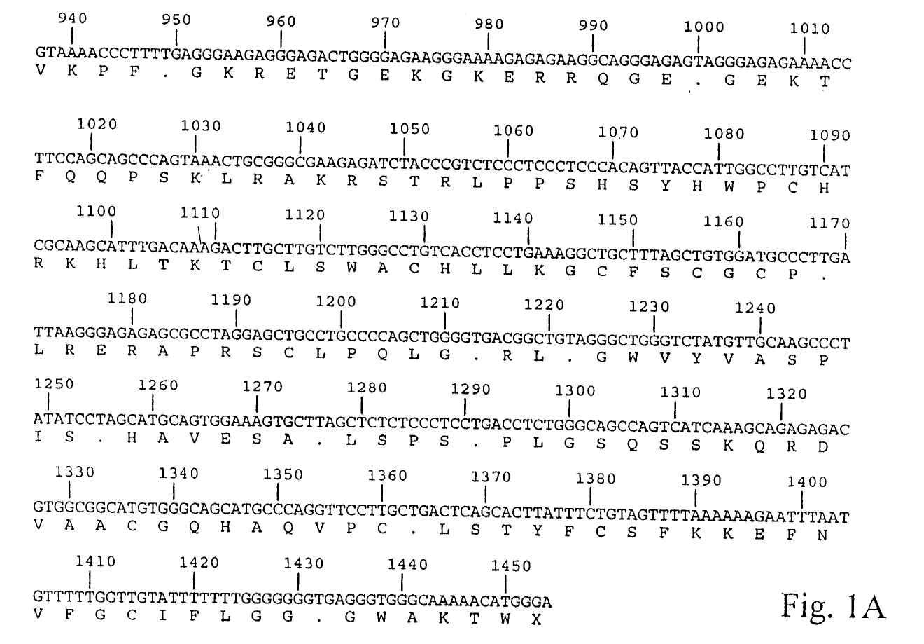

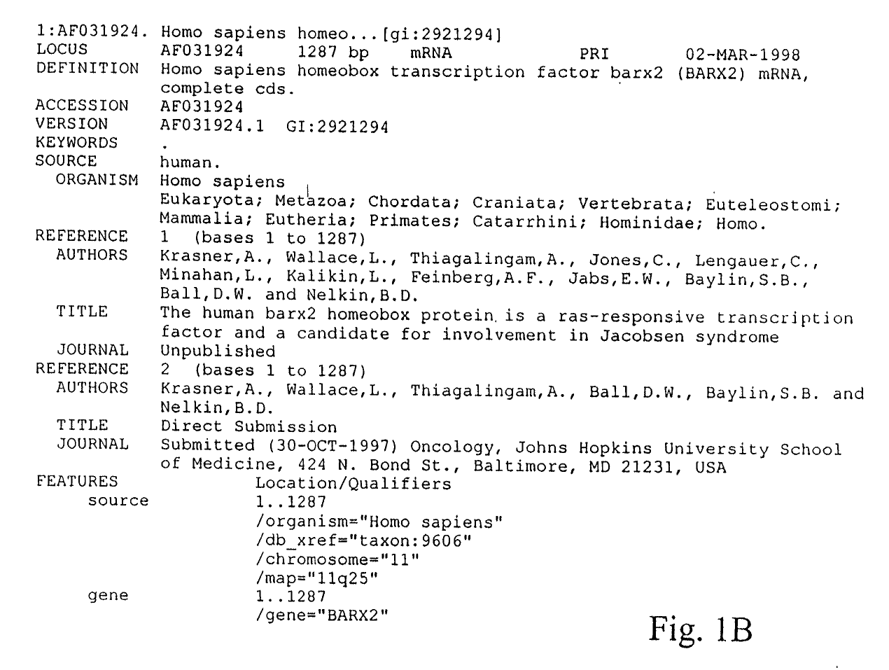

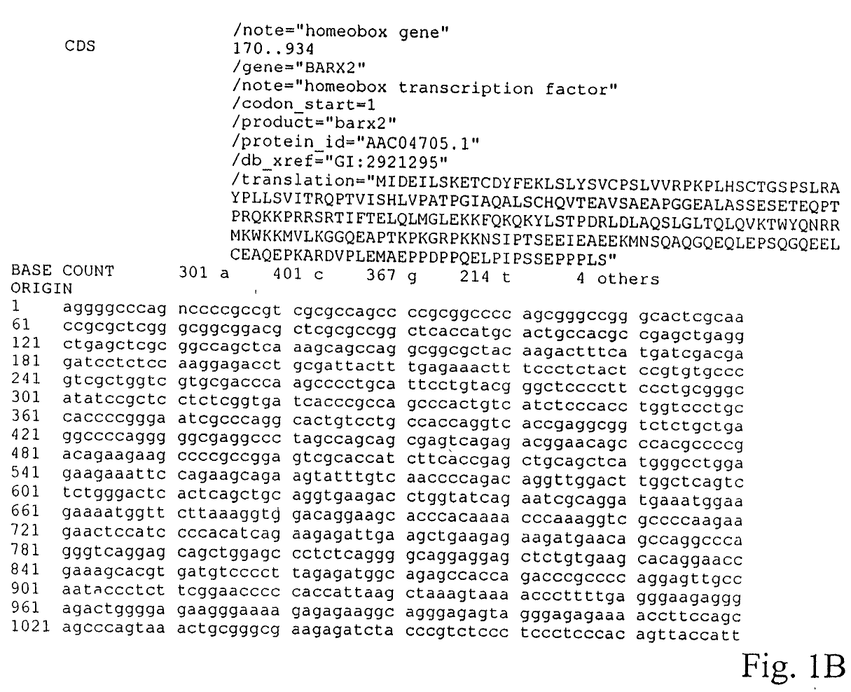

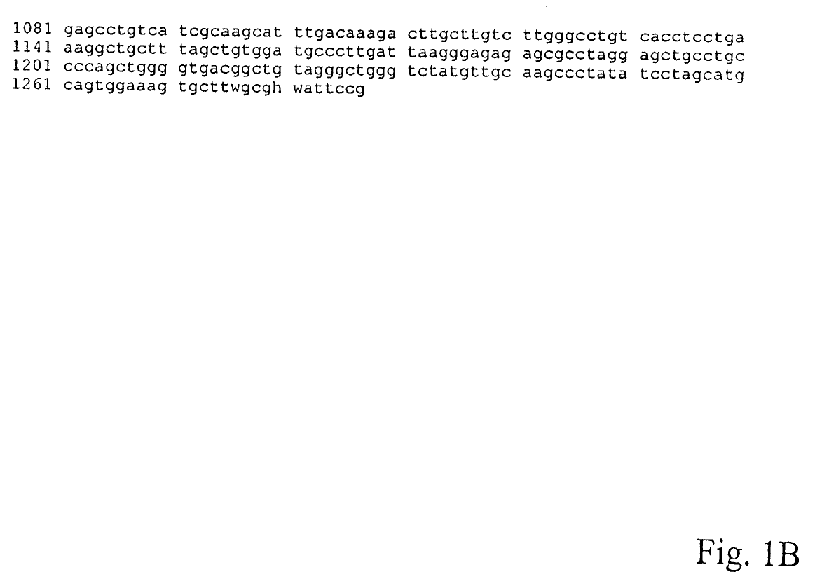

Barx2 cDNA sequence is publicly available from GenBank under Accession Nos NM003658 and AF031924. These sequences are also shown in FIGS. 1a and 1b. Further sequences for Barx2 in various species are available from GenBank under the following Accession Nos: AF265552 (sheep); NM_{—}013800 (Mus musculus); AH008405 (Homo sapiens); AF171222 (Homo sapiens, exon 4); AF171221 (Homo sapiens, exon 3); AF171220 (Homo sapiens, exon 2); AF171219 (Homo sapiens, exon 1); L77900 (Mus Musculus); AJ243512 (Homo sapiens); AI792204 (Homo sapiens); AI763040 (Homo sapiens).

In any event, a Barx2 cDNA may be readily obtained from a human cDNA library using well known techniques and portions of the genomic clones, or portions of the Barx2 cDNA sequence shown in FIGS. 1a and 1b, as a probe. A suitable human cDNA library is one prepared from mRNA isolated from a human ovary or human ovarian tissue or from a human ovarian cell line or from medullary thyroid carcinoma. Once a Barx2 cDNA or gene or fragment thereof has been identified as said, its nucleotide sequence may readily be determined, for example using Sanger dideoxy sequencing or other methods well known in the art.

It will be appreciated (and as is described in more detail in the Examples) is that the Barx2 gene may exist as a “wild-type” gene or it may exist as mutant alleles which differ in sequence to the wild-type gene. By “mutant alleles” is included not only sequences which lead to changes in function or expression of the Barx2 polypeptide, but allelic variants (or polymorphisms) which have no or only minor effect on the function or expression of the Barx2 polypeptide. Thus, the nucleic acids which selectively hybridise in the methods of the invention include those that selectively hybridise to the wild-type Barx2 gene sequence or to the wild-type Barx2 cDNA sequence (or mRNA sequence) as well as those which selectively hybridise to mutant alleles thereof. Also, it will readily be appreciated that, as is described in more detail herein, the skilled person can readily identify mutant alleles of the Barx2 gene and polymorphisms thereof. By “change in expression of the Barx2 polypeptide” is included any changes in the Barx2 gene which lead to changes in expression of the Barx2 polypeptide. For example, changes in the transcription of the Barx2 gene will lead to changes in the expression of the Barx2 polypeptide. Similarly, changes in the translation of Barx2 mRNA will lead to changes in the expression of the Barx2 polypeptide.

The amino acid sequences for Barx2 given in FIGS. 1(a) and 1(b) differ in that the FIG. 1(a) sequence extends at the N-terminus compared to that shown in FIG. 1(b). It is believed that either polypeptide may be useful in the invention (and is encoded by the Barx2 gene according to the invention) and that variants of either that retain useful activity, such as tumour suppressor activity, are useful in the practice of the invention. Both sequences are considered to be Barx2.

Mutation of the protein coding sequence of Barx2 may lead to a loss of function of the Barx2 protein; similarly, loss of function may be due to transcriptional silencing of the Barx2 gene or the presence of dominant negative mutations.

It will be appreciated that the methods of the invention defined above may involve either directly or indirectly comparing the results from the test sample with results from a control sample such as from a known non-cancerous (normal) sample or from a known cancerous sample. It will be appreciated that the nucleic acids which are useful in the method of the invention may readily be defined as those which selectively hybridise to the human-derived DNA of PAC1, or which selectively hybridise to Barx2 cDNA, or a mutant allele thereof, or their complement. In addition, the methods of the invention include the use of a nucleic acid which selectively hybridises to the Barx2 gene or cDNA, or mutant alleles thereof whatever the source of the gene or cDNA.

By “selectively hybridising” is meant that the nucleic acid has sufficient nucleotide sequence similarity with the said human DNA or cDNA that it can hybridise under moderately or highly stringent conditions. As is well known in the art, the stringency of nucleic acid hybridization depends on factors such as length of nucleic acid over which hybridisation occurs, degree of identity of the hybridizing sequences and on factors such as temperature, ionic strength and CG or AT content of the sequence. Thus, any nucleic acid which is capable of selectively hybridising as said is useful in the practice of the invention.

Nucleic acids which can selectively hybridise to the said human DNA or cDNA include nucleic acids which have >95% sequence identity, preferably those with >98%, more preferably those with >99% sequence identity, over at least a portion of the nucleic acid with the said human is DNA or cDNA. As is well known, human genes usually contain introns such that, for example, a mRNA or cDNA derived from a gene within the said human DNA would not match perfectly along its entire length with the said human DNA but would nevertheless be a nucleic acid capable of selectively hybridising to the said human DNA. Thus, the invention specifically includes nucleic acids which selectively hybridise to a Barx2 cDNA but may not hybridise to a Barx2 gene, or vice versa. For example, nucleic acids which span the intron-exon boundaries of the Barx2 gene may not be able to selectively hybridise to the Barx2 cDNA.

Typical moderately or highly stringent hybridisation conditions which lead to selective hybridisation are known in the art, for example those described in Molecular Cloning, a laboratory manual, 2nd edition, Sambrook et al (eds), Cold Spring Harbor Laboratory Press, Cold Spring Harbor, N.Y., USA, incorporated herein by reference.

An example of a typical hybridisation solution when a nucleic acid is immobilised on a nylon membrane and the probe nucleic acid is ≧500 bases or base pairs is:

6×SSC (saline sodium citrate)

0.5% sodium dodecyl sulphate (SDS)

100 μg/ml denatured, fragmented salmon sperm DNA

The hybridisation is performed at 68° C. The nylon membrane, with the nucleic acid immobilised, may be washed at 68° C. in 1×SSC or, for high stringency, 0.1×SSC.

20×SSC may be prepared in the following way. Dissolve 175.3 g of NaCl and 88.2 g of sodium citrate in 800 ml of H₂O. Adjust the pH to 7.0 with a few drops of a 10 N solution of NaOH. Adjust the volume to 1 liter with H₂O. Dispense into aliquots. Sterilize by autoclaving.

An example of a typical hybridisation solution when a nucleic acid is immobilised on a nylon membrane and the probe is an oligonucleotide of between 15 and 50 bases is:

3.0 M trimethylammonium chloride (TMACl)

0.01 M sodium phosphate (pH 6.8)

1 mm EDTA (pH 7.6)

0.5% SDS

100 μg/ml denatured, fragmented salmon sperm DNA

0.1% nonfat dried milk

The optimal temperature for hybridization is usually chosen to be 5° C. below the Tᵢ for the given chain length. Tᵢ is the irreversible melting temperature of the hybrid formed between the probe and its target sequence. Jacobs et al (1988) Nucl. Acids Res. 16, 4637 discusses the determination of Tᵢs. The recommended hybridization temperature for 17-mers in 3 M TMACl is 48-50° C.; for 19-mers, it is 55-57° C.; and for 20-mers, it is 58-66° C.

By “nucleic acid which selectively hybridises” is also included nucleic acids which will amplify DNA from the said region of human DNA by any of the well known amplification systems such as those described in more detail below, in particular the polymerase chain reaction (PCR). Suitable conditions for PCR amplification include amplification in a suitable 1×amplification buffer:

10×amplification buffer is 500 mM KCl; 100 mM Tris.Cl (pH 8.3 at room temperature); 15 mM MgCl₂; 0.1% gelatin.

A suitable denaturing agent or procedure (such as heating to 95° C.) is used in order to separate the strands of double-stranded DNA.

Suitably, the annealing part of the amplification is between 37° C. and 60° C., preferably 50° C.

Although the nucleic acid which is useful in the methods of the invention may be RNA or DNA, DNA is preferred. Although the nucleic acid which is useful in the methods of the invention may be double-stranded or single-stranded, single-stranded nucleic acid is preferred under some circumstances such as in nucleic acid amplification reactions.

The nucleic acid which is useful in the methods of the invention may be very large, such as 100 kb, if it is double stranded. For example, such large nucleic acids are useful as a template for making probes for use in FISH (fluorescence in situ hybridization) analysis. Typically, the labelled probes used in FISH are generally made by nick-translation or random priming from a genomic clone (such as an insert in a suitable PAC clone). Once made these probes are around 50-1000 nucleotides in length. The human DNA insert of PAC1, which may be a useful probe in its own lo right, contains exons 1 to 4 of Barx2. It is more preferably used as a template for nick-translation or random primer extension as described above. However, for certain diagnostic, probing or amplifying purposes, it is preferred if the nucleic acid has fewer than 10 000, more preferably fewer than 1000, more preferably still from 10 to 100, and in further preference from 15 to 30 base pairs (if the nucleic acid is double-stranded) or bases (if the nucleic acid is single stranded). As is described more fully below, single-stranded DNA primers, suitable for use in a polymerase chain reaction, are particularly preferred.

The nucleic acid for use in the methods of the invention is a nucleic acid capable of hybridising to the Barx2 gene or the Barx2 cDNA or mRNA or a mutant thereof. Fragments and variants of this gene, and cDNAs derivable from the mRNA encoded by the gene are also preferred nucleic acids for use in the methods of the invention.

Clearly nucleic acids which selectively hybridise to the gene itself or variants thereof are particularly useful. Fragments of the gene are preferred for use in the method of the invention. Fragments may be made by enzymatic or chemical degradation of a larger fragment, or may be chemically synthesised. By “gene” is included not only the introns and exons but also regulatory regions associated with, and physically close to, the introns and exons, particularly those 5′ to the 5′-most exon. By “physically close” is meant within 50 kb, preferably within 10 kb, more preferably within 5 kb and still more preferably within 2 kb. It is believed that the basic promoter and regulatory elements of the Barx2 gene probably lie up to 200-400 base pairs of the transcriptional start site or start of the coding region. However, tissue specific or inducible elements may be 50 kb in either direction of the coding regions (exons) or may be in the introns. Such elements of the Barx2 gene may be identified or located by DNAse hypersensitivity sites (detected on Southern blots) which indicate sites of regulatory protein binding. Alternatively, reporter constructs may be generated using the upstream genomic DNA (ie upstream of the 5′-most exon) and, for example, β-galactosidase as a reporter enzyme. Serial deletions and footprinting techniques may also be used to identify the regulatory regions.

By “fragment” of a gene is included any portion of the gene of at least 15 nucleotides in length (whether single stranded or double stranded) but more preferably the fragment is at least 20 nucleotides in length, most preferably at least 50 nucleotides in length and may be at least 100 nucleotides in length or may be at least 500 nucleotides in length. Preferably the fragment is no more than 50 kb and, more preferably, no more than 100 kb.

By “variant” of a gene is included specifically a cDNA, whether partial or full length, or whether copied from any splice variants of mRNA. We also include specifically a nucleic acid wherein, compared to the natural gene, nucleotide substitutions (including inversions), insertions and deletions are present whether in the gene or a fragment thereof or in a cDNA. Both variants and fragments will be selected according to their intended purposes; for probing, amplifying or diagnostic purposes, shorter fragments but with a greater degree of sequence identity (eg at least 80%, 90%, 95% or 99%) will generally be required.

It is particularly preferred if the nucleic acid for use in the methods of the invention is an oligonucleotide primer which can be used to amplify a portion of the gene or cDNA.

Preferred nucleic acids for use in the invention are those that selectively hybridise to the Barx2 gene or cDNA and do not hybridise to other genes or cDNAs. Such selectively hybridising nucleic acids can be readily obtained, for example, by reference to whether or not they hybridise to the Barx2 cDNA as described in FIGS. 1a and 1b.

The methods are suitable in respect of any cancer but it is preferred if the cancer is cancer of the ovary, colorectal, or other common adenocarcinomas such as cancer of the breast, lung and upper gastrointestinal tract. The methods are particularly suitable in respect of cancer of the ovary or colon; and the methods are most suitable in respect of ovarian cancer. It will be appreciated that the methods of the invention include methods of prognosis and methods which aid diagnosis. It will also be appreciated that the methods of the invention are useful to the physician or surgeon in determining a course of management or treatment of the patient.

Although it is believed that any sample containing nucleic acid derived from the patient is useful in the methods of the invention, since mutations in the Barx2 gene may occur in familial cancers and not just sporadic cancers, it is, however, preferred if the nucleic acid is derived from a sample of the tissue in which cancer is suspected or in which cancer may be or has been found. For example, if the tissue in which cancer is suspected or in which cancer may be or has been found is ovary, it is preferred if the sample containing nucleic acid is derived from the ovary of the patient. Samples of ovary may be obtained by surgical excision, laproscopy and biopsy, endoscopy and biopsy, and image-guided biopsy. The image may be generated by ultrasound or technetium-99-labelled antibodies or antibody fragments which bind or locate selectively at the ovary. The well known monoclonal antibody HMFG1 is a suitable antibody for imaging ovarian cancer. Ascites/peritoneal cavity fluid, and peritoneal samples, may be obtained by surgery or laproscopy. Similarly, if the tissue in which cancer is suspected or in which cancer may be or has been found is colon, it is preferred if the sample containing nucleic acid is derived from the colon of the patient; and so on. Colon samples may be obtained by colonoscopy.

Other samples in which it may be beneficial to analyse Barx2 include lymph nodes, blood, serum and potential or actual sites of metastasis, for example bone.

The sample may be directly derived from the patient, for example, by biopsy of the tissue, or it may be derived from the patient from a site remote from the tissue, for example because cells from the tissue have migrated from the tissue to other parts of the body. Alternatively, the sample may be indirectly derived from the patient in the sense that, for example, the tissue or cells therefrom may be cultivated in vitro, or cultivated in a xenograft model; or the nucleic acid sample may be one which has been replicated (whether in vitro or in vivo) from nucleic acid from the original source from the patient. Thus, although the nucleic acid derived from the patient may have been physically within the patient, it may alternatively have been copied from nucleic acid which was physically within the patient. The tumour tissue may be taken from the primary Tumour or from metastases.

It will be appreciated that a useful method of the invention includes the analysis of mutations in, or the detection of the presence or absence of, the Barx2 gene in any suitable sample. The sample may suitably be a freshly-obtained sample from the patient, or the sample may be an historic sample, for example a sample held in a library of samples.

Certain mutations in the Barx2 gene which are believed to be associated with cancer are described in the Examples.

Conveniently, the nucleic acid, capable of selectively hybridising to the said human DNA and which is used in the methods of the invention further comprises a detectable label.

By “detectable label” is included any convenient radioactive label such as ³²P, ³³P or ³⁵S which can readily be incorporated into a nucleic acid molecule using well known methods; any convenient fluorescent or chemiluminescent label which can readily be incorporated into a nucleic acid is also included. In addition the term “detectable label” also includes a moiety which can be detected by virtue of binding to another moiety (such as biotin which can be detected by binding to streptavidin); and a moiety, such as an enzyme, which can be detected by virtue of its ability to convert a colourless compound into a coloured compound, or vice versa (for example, alkaline phosphatase can convert colourless o-nitrophenylphosphate into coloured o-nitrophenol). Conveniently, the nucleic acid probe may occupy a certain position in a fixed assay and whether the nucleic acid hybridises to the said region of human DNA can be determined by reference to the position of hybridisation in the fixed assay. The detectable label may also be a fluorophore-quencher pair as described in Tyagi & Kramer (1996) Nature Biotechnology 14, 303-308.

It will be appreciated that the aforementioned methods may be used for presymptomatic screening of a patient who is in a risk group for cancer. High risk patients for screening include patients over 50 years of age or patients who carry a gene resulting in increased susceptibility (eg predisposing versions of BRCA1, BRCA2 or p53); patients with a family history of breast/ovarian cancer; patients with affected siblings; nulliparous women; and women who have a long interval between menarche and menopause. Similarly, the methods may be used for the pathological classification of tumours such as ovarian tumours or colon tumours.

Conveniently, in the methods of the first, second and third aspects of the invention the nucleic acid which is capable of the said selective hybridisation (whether labelled with a detectable label or not) is contacted with a nucleic acid derived from the patient under hybridising conditions. Suitable hybridising conditions include those described above.

It is preferred that if the sample containing nucleic acid derived from the patient is not a substantially pure sample of the tissue or cell type in question that the sample is enriched for the said tissue or cells. For example, enrichment for ovarian cells in a sample such as a blood sample may be achieved using, for example, cell sorting methods such as fluorescent activated cell sorting (FACS) using an ovary cell-selective antibody, or at least an antibody which is selective for an epithelial cell. For example, Cam 5.2, anticytokeratin ⅞, from Becton Dickinson, 2350 Qume Drive, San Jose, Calif., USA, may be useful. The source of the said sample also includes biopsy material as discussed above and tumour samples, also including fixed paraffin mounted specimens as well as fresh or frozen tissue. The nucleic acid sample from the patient may be processed prior to contact with the nucleic acid which selectively hybridises to Barx2. For example, the nucleic acid sample from the patient may be treated by selective amplification, reverse transcription, immobilisation (such as sequence specific immobilisation), or incorporation of a detectable marker.

It is particularly preferred if the methods of the invention include the determination of mutations in, or the detection of the presence or absence of, the Barx2 gene. Mutations in the Barx2 gene found in cancer cells are described in the Examples. In particular, missense mutations have been detected which introduce a Ser→Pro or an Ala→Pro change in the Barx2 polypeptide.

The methods of the first, second and third aspects of the invention may involve sequencing of DNA at one or more of the relevant positions within the relevant region, including direct sequencing; direct sequencing of PCR-amplified exons; differential hybridisation of an oligonucleotide probe designed to hybridise at the relevant positions within the relevant region (conveniently this uses immobilised oligonucleotide probes in, so-called, “chip” systems which are well known in the art); denaturing gel electrophoresis following digestion with an appropriate restriction enzyme, preferably following amplification of the relevant DNA regions; S1 nuclease sequence analysis; non-denaturing gel electrophoresis, preferably following amplification of the relevant DNA regions; conventional RFLP (restriction fragment length polymorphism) assays; heteroduplex analysis; selective DNA amplification using oligonucleotides; fluorescent in-situ hybridisation (FISH) of interphase chromosomes; ARMS-PCR (Amplification Refractory Mutation System-PCR) for specific mutations; cleavage at mismatch sites in hybridised nucleic acids (the cleavage being chemical or enzymic); SSCP single strand conformational polymorphism or DGGE (discontinuous or denaturing gradient gel electrophoresis); analysis to detect mismatch in annealed normal/mutant PCR-amplified DNA; and protein truncation assay (translation and transcription of exons —if a mutation introduces a stop codon a truncated protein product will result). Other methods may be employed such as detecting changes in the secondary structure of single-stranded DNA resulting from changes in the primary sequence, for example using the cleavase I enzyme. This system is commercially available from GibcoBRL, Life Technologies, 3 Fountain Drive, Inchinnan Business Park, Paisley PA4 9RF, Scotland.

It will be appreciated that the methods of the invention may also be carried out on “DNA chips”. Such “chips” are described in U.S. Pat. No. 5,445,934 (Affymetrix; probe arrays), WO 96/31622 (Oxford; probe array plus ligase or polymerase extension), and WO 95/22058 (Affymax; fluorescently marked targets bind to oligomer substrate, and location in array detected); all of these are incorporated herein by reference.

Detailed methods of mutation detection are described in “Laboratory Protocols for Mutation Detection” 1996, ed. Landegren, Oxford University Press on behalf of HUGO (Human Genome Organisation).

It is preferred if RFLP is used for the detection of fairly large (≧500 bp) deletions or insertions. Southern blots may be used for this method of the invention.

PCR amplification of smaller regions (maximum 300 bp) to detect small changes greater than 3-4 bp insertions or deletions may be preferred. Amplified sequence may be analysed on a sequencing gel, and small changes (minimum size 3-4 bp) can be visualised. Suitable primers are designed as herein described.

In addition, using either Southern blot analysis or PCR restriction enzyme variant sites may be detected. For example, for analysing variant sites in genomic DNA restriction enzyme digestion, gel electrophoresis, Southern blotting, and hybridisation specific probe (for example any suitable fragment derived from the Barx2 cDNA or gene).

For example, for analysing variant sites using PCR DNA amplification, restriction enzyme digestion, gel detection by ethidium bromide, silver staining or incorporation of radionucleotide or fluorescent primer in the PCR.

Other suitable methods include the development of allele specific oligonucleotides (ASOs) for specific mutational events. Similar methods are used on RNA and cDNA for the suitable tissue, such as ovarian or breast tissue.

Whilst it is useful to detect mutations in any part of the Barx2 gene, it is preferred if the mutations are detected in the exons of the gene and it is further preferred if the mutations are ones which change the coding sense. The detection of these mutations is a preferred aspect of the invention. Similarly, the invention also includes probes and primers and other means for detecting the specific mutations identified in the Examples, all of which can be designed, made and used by methods well known to the skilled person.

The methods of the invention also include checking for loss-of-heterozygosity (LOH; shows one copy lost). LOH may be a sufficient marker for diagnosis; looking for mutation/loss of the second allele may not be necessary. LOH of the gene may be detected using polymorphisms in the coding sequence, and introns, of the gene. LOH in a tumour cell, from whatever source, compared to blood is useful as a diagnostic tool, eg it may show that the tumour has progressed and requires more stringent treatment.

Particularly preferred nucleic acids for use in the aforementioned methods of the invention are those selected from the group consisting of primers suitable for amplifying nucleic acid.

Suitably, the primers are selected from the group consisting of primers which hybridise to the nucleotide sequences shown in any of the Figures which show Barx2 gene or cDNA sequences. It is particularly preferred if the primers hybridise to the introns of the Barx2 gene or if the primers are ones which will prime synthesis of DNA from the Barx2 gene or cDNA but not from other genes or cDNAs.

Primers which are suitable for use in a polymerase chain reaction (PCR; Saiki et al (1988) Science 239, 487491) are preferred. Suitable PCR primers may have the following properties:

It is well-known that the sequence at the 5′ end of the oligonucleotide need not match the target sequence to be amplified.

It is usual that the PCR primers do not contain any complementary structures with each other longer than 2 bases, especially at their 3′ ends, as this feature may promote the formation of an artifactual product called “primer dimer”. When the 3′ ends of the two primers hybridize, they form a “primed template” complex, and primer extension results in a short duplex product called “primer dimer”.

Internal secondary structure should be avoided in primers. For symmetric PCR, a 40-60% G+C content,is often recommended for both primers, with no long stretches of any one base. The classical melting temperature calculations used in conjunction with DNA probe hybridization studies often predict that a given primer should anneal at a specific temperature or that the 72° C. extension temperature will dissociate the primer/template hybrid prematurely. In practice, the hybrids are more effective in the PCR process than generally predicted by simple Tₘ calculations.

Optimum annealing temperatures may be determined empirically and may be higher than predicted. Taq DNA polymerase does have activity in the 37-55° C. region, so primer extension will occur during the annealing step and the hybrid will be stabilized. The concentrations of the primers are equal in conventional (symmetric) PCR and, typically, within 0.1- to 1-μM range.

Any of the nucleic acid amplification protocols can be used in the method of the invention including the polymerase chain reaction, QB replicase and ligase chain reaction. Also, NASBA (nucleic acid sequence based amplification), also called 3SR, can be used as described in Compton (1991) Nature 350, 91-92 and AIDS (1993), Vol 7 (Suppl 2), S108 or SDA (strand displacement amplification) can be used as described in Walker et al (1992) Nucl. Acids Res. 20, 1691-1696. The polymerase chain reaction is particularly preferred because of its simplicity.

When a pair of suitable nucleic acids of the invention are used in a PCR it is convenient to detect the product by gel electrophoresis and ethidium bromide staining. As an alternative to detecting the product of DNA amplification using agarose gel electrophoresis and ethidium bromide staining of the DNA, it is convenient to use a labelled oligonucleotide capable of hybridising to the amplified DNA as a probe. When the amplification is by a PCR the oligonucleotide probe hybridises to the interprimer sequence as defined by the two primers. The oligonucleotide probe is preferably between 10 and 50 nucleotides long, more preferably between 15 and 30 nucleotides long. The probe may be labelled with a radionuclide such as ³²P, ³³P and ³⁵S using standard techniques, or may be labelled with a fluorescent dye. When the oligonucleotide probe is fluorescently labelled, the amplified DNA product may be detected in solution (see for example Balaguer et al (1991) “Quantification of DNA sequences obtained by polymerase chain reaction using a bioluminescence adsorbent” Anal. Biochem. 195, 105-110 and Dilesare et al (1993) “A high-sensitivity electrocheniluminescence-based detection system for automated PCR product quantitation” BioTechniques 15, 152-157.

PCR products can also be detected using a probe which may have a fluorophore-quencher pair or may be attached to a solid support or may have a biotin tag or they may be detected using a combination of a capture probe and a detector probe.

Fluorophore-quencher pairs are particularly suited to quantitative measurements of PCR reactions (eg RT-PCR). Fluorescence polarisation using a suitable probe may also be used to detect PCR products.

Oligonucleotide primers can be synthesised using methods well known in the art, for example using solid-phase phosphoramidite chemistry.

The present invention provides the use of a nucleic acid which selectively hybridises to the human-derived DNA of PAC1 as described herein or to the Barx2 gene, or a mutant allele thereof, or a nucleic acid which selectively hybridises to Barx2 cDNA or a mutant allele thereof, or their complement in a method of diagnosing cancer or prognosing cancer or determining susceptibility to cancer; or in the manufacture of a reagent for carrying out these methods.

Also, the present invention provides a method of determining the presence or absence, or mutation in, the said Barx2 gene. Preferably, the method uses a suitable sample from a patient.

The methods of the invention include the detection of mutations in the Barx2 gene.

The methods of the invention may make use of a difference in restriction enzyme cleavage sites caused by mutation. A non-denaturing gel may be used to detect differing lengths of fragments resulting from digestion with an appropriate restriction enzyme.

An “appropriate restriction enzyme” is one which will recognise and cut the wild-type sequence and not the mutated sequence or vice versa. The sequence which is recognised and cut by the restriction enzyme (or not, as the case may be) can be present as a consequence of the mutation or it can be introduced into the normal or mutant allele using mismatched oligonucleotides in the PCR reaction. It is convenient if the enzyme cuts DNA only infrequently, in other words if it recognises a sequence which occurs only rarely.

In another method, a pair of PCR primers are used which match (ie hybridise to) either the wild-type genotype or the mutant genotype but not both. Whether amplified DNA is produced will then indicate the wild-type or mutant genotype (and hence phenotype). However, this method relies partly on a negative result (ie the absence of amplified DNA) which could be due to a technical failure. It therefore may be less reliable and/or requires additional control experiments.

A preferable method employs similar PCR primers but, as well as hybridising to only one of the wild-type or mutant sequences, they introduce a restriction site which is not otherwise there in either the wild-type or mutant sequences.

The nucleic acids which selectively hybridise to the Barx2 gene or cDNA, or which selectively hybridise to the genomic clones containing Barx2 as described in the Examples are useful for a number of purposes. They can be used in Southern hybridization to genomic DNA and in the RNase protection method for detecting point mutations already discussed above. The probes can be used to detect PCR amplification products. They may also be used to detect mismatches with the Barx2 gene or mRNA in a sample using other techniques. Mismatches can be detected using either enzymes (eg S1 nuclease or resolvase), chemicals (eg hydroxylamine or osmium tetroxide and piperidine), or changes in electrophoretic mobility of mismatched hybrids as compared to totally matched hybrids. These techniques are known in the art. Generally, the probes are complementary to the Barx2 gene coding sequences, although probes to certain introns are also contemplated. A battery of nucleic acid probes may be used to compose a kit for detecting loss of or mutation in the wild-type Barx2 gene. The kit allows for hybridization to the entire Barx2 gene. The probes may overlap with each other or be contiguous.

If a riboprobe is used to detect mismatches with mRNA, it is complementary to the mRNA of the human Barx2 gene. The riboprobe thus is an anti-sense probe in that it does not code for the protein encoded by the Barx2 gene because it is of the opposite polarity to the sense strand. The riboprobe generally will be labelled, for example, radioactively labelled which can be accomplished by any means known in the art. If the riboprobe is used to detect mismatches with DNA it can be of either polarity, sense or anti-sense. Similarly, DNA probes also may be used to detect mismatches.

Nucleic acid probes may also be complementary to mutant alleles of the Barx2 gene. These are useful to detect similar mutations in other patients on the basis of hybridization rather than mismatches. As mentioned above, the Barx2 gene probes can also be used in Southern hybridizations to genomic DNA to detect gross chromosomal changes such as deletions and insertions.

According to the diagnostic and prognostic method of the present invention, loss of, or modification of, the wild-type gene function may be detected. The loss may be due to either insertional, deletional or point mutational events. If only a single allele is mutated, an early neoplastic state may be indicated. However, if both alleles are mutated then a malignant state is indicated or an increased probability of malignancy is indicated. The finding of such mutations thus provides both diagnostic and prognostic information. A Barx2 gene allele which is not deleted (eg that on the sister chromosome to a chromosome carrying a gene deletion) can be screened for other mutations, such as insertions, small deletions, and point mutations. We believe that detecting a mutation in a single copy (allele) of the gene is useful. Loss of the second allele may be necessary for carcinogenesis. If the second copy was lost routinely by a gross mechanism, this could be a useful event to detect. Some mutations of the gene may have a dominant negative effect on the remaining allele. Mutations leading to non-functional gene products may also lead to a malignant state or an increased probability of malignancy. Mutational events (such as point mutations, deletions, insertions and the like) may occur in regulatory regions, such as in the promoter of the gene, leading to loss or diminution of expression of the mRNA. Point mutations may also abolish proper RNA processing, leading to loss of or alteration in the expression of the Barx2 gene product or to the Barx2 polypepide being non-functional or having an altered expression. It is preferred if the amount of Barx2 mRNA in a test sample is quantified and compared to that present in a control sample. It is also preferred if the splicing patterns or structure of Barx2 mRNA in a test sample is determined and compared to that present in a control sample. However, the detection of altered Barx2 expression is less preferred.

The gene has two alleles, and it will be appreciated that alterations to both alleles may have a greater effect on cell behaviour than alteration to one. It is expected that at least one mutant allele has mutations which result in an altered coding sequence. Modifications to the second allele, other than to the coding sequence, may include total or partial gene deletion, and loss or mutation of regulatory regions.

The amount of Barx2 mRNA is suitably determined per unit mass of sample tissue or per unit number of sample cells and compared this to the unit mass of known normal tissue or per unit number of normal cells. RNA may be quantitated using, for example, northern blotting or quantitative RT-PCR.

The invention also includes the following methods: in vitro transcription and translation of Barx2 gene to identify truncated gene products, or altered properties such as substrate binding; immunohistochemistry of tissue sections to identify cells in which expression of the protein is reduced/lost, or its distribution is altered within cells or on their surface; and the use of RT-PCR using random primers, prior to detection of mutations in the region as described above. It is preferred if altered distribution of the Barx2 polypeptide is screened for.

The methods of the inventions also include detection of inactivation of the Barx2 gene by investigating its DNA methylation status. DNA methylation of the Barx2 gene can be assessed using standard techniques such as those described in Herman et al (1996) Proc. Natl. Acad. Sci. USA 93, 9821-9826. Aberrant methylation of the Barx2 gene may be associated with its inactivation.

The Examples show that there is a correlation between the methylation status of the Barx2 gene and its level of expression: down-regulation of Barx2 expression correlates with Barx2 methylation. Thus, the invention includes methods of determining the level of expression of Barx2 by assessing the level or extent of methylation of the Barx2 gene, and of using this information to determining susceptibility, diagnose or predict the relative prospects of a particular outcome of a cancer in a patient.

Thus, a further aspect of the invention provides a method for determining the susceptibility of a patient to cancer comprising the steps of

(i) obtaining a sample containing the Barx2 gene from the patient;

(i) determining the level of methylation of the Barx2 gene;

(iii) comparing the level of methylation of the Barx2 gene from the patient sample with the level of methylation in a non-tumorous sample; and

(iv) if the patient sample has a higher degree of methylation of the Barx2 gene compared to the non-tumorous sample this is indicative of susceptibility to cancer.

A still further aspect of the invention provides a method of diagnosing cancer in a patient comprising the steps of

(i) obtaining a sample containing the Barx2 gene from the patient;

(i) determining the level of methylation of the Barx2 gene;

(iii) comparing the level of methylation of the Barx2 gene from the patient sample with the level of methylation in a non-tumorous sample; and

(iv) if the patient sample has a higher degree of methylation of the Barx2 gene compared to the non-tumorous sample this is indicative of cancer.

A yet still further aspect of the,invention provides a method of predicting the relative prospects of a particular outcome of a cancer patient comprising the steps of

(i) obtaining a sample containing the Barx2 gene from the patient;

(i) determining the level of methylation of the Barx2 gene;

(iii) comparing the level of methylation of the Barx2 gene from the patient sample with the level of methylation in a non-tumorous sample; and

(iv) if the patient sample has a higher degree of methylation of the Barx2 gene compared to the non-tumorous sample this is indicative of a lower chance of a successful outcome.

Methods for determining methylation differences between nucleic acids are well known in the art and include (a) the use of methylation sensitive single nucleotide primer extension (Ms-SNuPE); (b) digestion of genomic DNA with methylation sensitive restriction enzymes by Southern analysis; and (c) PCR-based methylation assays utilizing digestion of genomic DNA with methylation-sensitive restriction enzymes prior to PCR amplification. The above methods may be carried out following the digestion or bisulphite-converted DNA. Bisulphite treatment causes unmethylated cytosine in the nucleic acid sample to be converted to uracil.

Typically, methylation of the promoter region of the Barx2 gene is analysed. The 5′ region of the Barx2 gene is described in Hjalt & Murray (1999) Genomics 62, 456-459. We believe that the sequence disclosed therein includes a CpG island, and that it includes all or most of the promoter region.

A further aspect of the invention provides a system (or it could also be termed a kit of parts) for detecting the presence or absence of, or mutation in, the relevant region of human DNA, the system comprising a nucleic acid capable of selectively hybridising to the relevant region of human DNA and a nucleoside triphosphate or deoxynucleoside triphosphate or derivative thereof. Preferred nucleic acids capable of selectively hybridising to the relevant region of human DNA are the same as those preferred above.

The “relevant region of human DNA” includes the Barx2 gene, the Barx2 cDNA and the human-derived DNA present in the genomic clones containing Barx2. Preferably, the relevant region of human DNA is the Barx2 gene as herein defined.

By “mutation” is included insertions, substitutions and deletions.

By “nucleoside triphosphate or deoxynucleoside triphosphate or derivative thereof” is included any naturally occurring nucleoside triphosphate or deoxynucleoside triophosphate such as ATP, GTP, CTP, and UTP, dATP dGTP, dCTP, TTP as well as non-naturally derivatives such as those that include a phosphorothioate linkage (for example αS derivatives).

Conveniently the nucleoside triphosphate or deoxynucleoside triphosphosphate is radioactively labelled or derivative thereof, for example with ³²P, ³³P or ³⁵S, or is fluorescently labelled or labelled with a chemiluminescence compound or with digoxygenin.

Conveniently deoxynucleotides are at a concentration suitable for dilution to use in a PCR.

Thus, the invention includes a kit of parts which includes a nucleic acid capable of selectively hybridising to the said relevant region of human DNA and means for detecting the presence or absence of, or a mutation in, the said region. Means for detecting the presence or absence of, or a mutation in, the said region include, for example, a diagnostic restriction enzyme or a mutant-specific nucleic acid probe or the like.

A further aspect of the invention provides a system for detecting the presence or absence of, or mutation in, the relevant region of DNA, the system comprising a nucleic acid which selectively hybridises to the relevant region of human DNA and a nucleic acid modifying enzyme. Preferred nucleic acids capable of selectively hybridising to the relevant region of human DNA are the same as those preferred above.

By “mutation” is included insertions, substitutions (including transversions) and deletions.

By “nucleic acid modifying enzyme” is included any enzyme capable of modifying an RNA or DNA molecule.

Preferred enzymes are selected from the group consisting of DNA polymerases, DNA ligases, polynucleotide kinases or restriction endonucleases. A particularly preferred enzyme is a thermostable DNA polymerase such as Taq DNA polymerase. Nucleases such as Cleavase I which recognise secondary structure, for example mismatches; may also be useful.

Detecting mutations in the gene will be useful for determining the appropriate treatment for a patient, eg Barx2 gene therapy (see below). Detecting mutations in the gene may be useful to identify a subset of patients whose tumours have this shared characteristic, and can be analysed as a group for prognosis or response to various therapies.

As the gene appears to be a late event, detection of mutations in it may be useful for prognosis and determining what treatment may be most appropriate for the patient.

Mutations in the gene may be related to response or resistance to certain treatments, this may be investigated using cell lines with known sensitivity to various therapies, or by clinical correlation studies. As is described in detail in the Examples, the presence of a functional Barx2 gene appears to be associated with an increase in sensitivity (or reduction in resistance) to cisplatinum.

It is possible that the gene would be used as part of a panel of markers and tests, which the combined results of would direct therapy. Detecting mutations in the gene may be useful for monitoring disease spread and load.

Analysis of the gene may be useful for differential diagnosis in the case where mutations in the gene are common in one tumour, but not another. For example, secondary tumours of gastrointestinal origin are frequently found in the ovaries and are difficult to distinguish from tumours of true ovarian origin.

A further aspect of the invention provides a method for determining the susceptibility of a patient to cancer comprising the steps of (i) obtaining a sample containing protein derived from the patient; and (ii) determining the relative amount, or the intracellular location, or physical form, of the Barx2 polypeptide, or the relative activity of, or change in activity of, or altered activity of, the Barx2 polypeptide.

A still further aspect of the invention provides a method of diagnosing cancer in a patient comprising the steps of (i) obtaining a sample containing protein derived from the patient; and (ii) determining the relative amount, or the intracellular location, or physical form, of the Barx2 polypeptide, or the relative activity of, or change in activity of, or altered activity of, the Barx2 polypeptide.

A yet still further aspect of the invention provides a method of predicting the relative prospects of a particular outcome of a cancer in a patient comprising the steps of (i) obtaining a sample containing protein derived from the patient; and (ii) determining the relative amount, or the intracellular location, or physical form of the Barx2 polypeptide, or the relative activity of, or change in activity of, or altered activity of, the Barx2 polypeptide.

It is believed that down regulation of Barx2 expression indicates a poorer prognosis than when Barx2 expression is not down-regulated. Down-regulation of Barx2 expression is also believed to indicate resistance to platinum drugs.

The methods of the invention also include the measurement and detection of the Barx2 polypeptide or mutants thereof in test samples and their comparison in a control sample. It may also be useful to detect altered activity of the polypeptide. It will be appreciated that the measurements taken with respect to Barx2 polypeptide (or mutants thereof) in the test sample may be compared to the equivalent measurements in control samples which may be derived from known non-cancerous (normal) cells or derived from known cancerous cells.

The sample containing protein derived from the patient is conveniently a sample of the tissue in which cancer is suspected or in which cancer may be or has been found These methods may be used for any cancer, but they are particularly suitable in respect of cancer of the ovary, colorectal cancer, and other common adenocarcinomas such as cancer of the breast, lung or upper gastrointestinal tract; the methods are especially suitable in respect of cancer of the ovary or colorectal cancer; the methods are most suitable in respect of ovarian cancer. Methods of obtaining suitable samples are described in relation to earlier methods.

The methods of the invention involving detection of the Barx2 polypeptide are particularly useful in relation to historical samples such as those containing paraffin-embedded sections of tumour samples.

The relative amount of, or the intracellular location of, or the physical form of, the Barx2 polypeptide may be determined in any suitable way. The polypeptide sequence of Barx2 is given in the GenBank data library under Accession Nos NM 003658 and AF 031924 (See FIGS. 1a and 1b).

It is preferred if the relative amount of, or intracellular location of, or physical form of the Barx2 polypeptide is determined using a molecule which selectively binds to Barx2 polypeptide or which selectively binds to a mutant form of Barx2 polypeptide. Suitably, the molecule which selectively binds to Barx2 or which selectively binds to a mutant of Barx2 is an antibody. The antibody may also bind to a natural variant or fragment of Barx2 polypeptide.

Antibodies to Barx2 can be made by methods well known in the art.

It is preferred if the antibodies used are selective for Barx2. By “selective for Barx2” we mean that they bind Barx2 but do not bind substantially to other polypeptides. Preferably the antibody binds selectively only to Barx2 polypeptide.

Antibodies which can selectively bind to a mutant form of Barx2 can be made, for example, by using peptides which encompass the changed amino acid or otherwise modified region of Barx2, or by using fusion proteins which express a portion of the Barx2 polypeptide which includes the changed amino acid or otherwise modified region.

In any case, based on the genetic code, it is possible to deduce readily the change in the amino acid sequence. Antibodies which are selective for a mutant Barx2 polypeptide as herein disclosed form a further aspect of the invention.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

By “the relative amount of Barx2 polypeptide” is meant the amount of Barx2 polypeptide per unit mass of sample tissue or per unit number of sample cells compared to the amount of Barx2 polypeptide per unit mass of known normal tissue or per unit number of normal cells. The relative amount may be determined using any suitable protein quantitation method. In particular, it is preferred if antibodies are used and that the amount of Barx2 is determined using methods which include quantititative western blotting, enzyme-linked immunosorbent assays (ELISA) or quantitative immunohistochemistry.

The neoplastic condition of lesions can also be detected on the basis of the alteration of wildtype Barx2 polypeptide. Such alterations can be determined by sequence analysis in accordance with conventional techniques. More preferably, antibodies (polyclonal or monoclonal) are used to detect differences in, or the absence of Barx2 polypeptide or peptides derived therefrom. The antibodies may be prepared as discussed herein.

Other techniques for raising and purifying antibodies are well known in the art and any such techniques may be chosen to achieve the preparations claimed in this invention. In a preferred embodiment of the invention, antibodies will immunoprecipitate Barx2 proteins from solution as well as react with Barx2 protein on Western or immunoblots of polyacrylamide gels. In another preferred embodiment, antibodies will detect Barx2 proteins in paraffin or frozen tissue sections, using immunocytochemical techniques.

Preferred embodiments relating to methods for detecting Barx2 or its mutations include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference.

The intracellular location of Barx2 may readily be determined using methods known in the art such as immunocytochemistry in which a labelled antibody (for example, radioactively or fluorescently labelled antibody) is used to bind to Barx2 and its location within the cell is determined microscopically. For example, it is possible using this methodology to determine whether the Barx2 is located in the cytoplasm or in the nucleus or, if located in both compartments, the proportion of Barx2 which is located in each compartment. A change in the location of Barx2 in a test sample compared to a non-cancerous, normal control sample may be indicative of a cancerous state.

Methods for detecting altered cellular distribution include immunohistochemistry (IHC; for example, where the antibody or a secondary antibody which recognises the first, is labelled with an enzyme, a fluorescent tag, a radioisotope), computer-aided image analysis of IHC stained sections; and flow cytometric analysis of cell nuclei released from fresh tissue or from paraffin sections.

The relative activity of Barx2 can be determined by measuring the activity of the Barx2 polypeptide per unit mass of sample tissue or per unit number of sample cells and comparing this activity to the activity of the Barx2 polypeptide per unit mass of known normal tissue or per unit number of normal cells. The relative amount may be determined using any suitable assay of Barx2 activity. Preferably, the assay is selective for the Barx2 polypeptide activity.

The invention also provides an antibody which reacts with a mutant Barx2 polypeptide or fragment thereof, wherein said mutant Barx2 is a mutant found in a cancer cell. Preferably, the antibody does not react with wild-type Barx2 polypeptide. Such antibodies are useful in the diagnostic assays and methods of the invention and may be made, for example, by using peptides whose sequence is derived from mutant Barx2 polypeptide as immunogens.

The invention also provides a nucleic acid which selectively hybridises to a nucleic acid encoding a mutant Barx2 polypeptide, which mutant is one found in a cancer cell. Such nucleic acids are useful in the diagnostic assays and methods of the invention.

It will be appreciated that in respect of the certain nucleic acid-based methods of diagnosis, determination of susceptibility and prediction of relative prospects of outcome, the methods involve determining whether the status of Barx2 nucleic acid (whether DNA or mRNA) is altered in a sample being tested compared to a sample-from an equivalent tissue or other source which is known to be normal or disease free.

Peptides based on the mutant sequences may be useful in stimulating an immune response.

The invention includes the ability to predict response to platinum (eg cisplatin) for first line therapy or for relapsed disease. Typically, Barx2 expression or the methylation status of the Barx2 gene is investigated. An increase in Barx2 expression compared to normal or non-tumorous cells, or a decrease in methylation of the Barx2 gene compared to normal or non-tumorous cells, indicates that the cells may be sensitive to platinum chemotherapy such as with cisplatin.

The expression of Barx2 can be detected by any convenient method, for example by immunohistochemistry. The methylation status of the Barx2 gene can be determined by any suitable method, for example by methylation PCR. Bisulfite PCR methods allow for methylation analysis to be carried out, for example using fixed biopsy specimens.

A further aspect of the invention provides a method of treating cancer comprising the step of administering to the patient a nucleic acid which selectively hybridises to the Barx2 gene, or a nucleic acid which hybridises selectively to Barx2 cDNA.

A further aspect of the invention provides a method of treating cancer comprising the step of administering to the patient a nucleic acid which encodes the Barx2 polypeptide or a functional variant or portion or fusion thereof.

The invention also includes the administration of all or part of the Barx2 gene or cDNA to a patient with a cancer. Preferably, the cancer to be treated in ovarian cancer or colorectal cancer.

Suitably, the nucleic acid which is administered to the patient is a nucleic acid which encodes the Barx2 polypeptide or a functional variant or portion thereof. Preferably, the Barx2 polypeptide is a wild-type polypeptide or a variant polypeptide which has substantially wild-type activities. It is less preferred if the Barx2 polypeptide is a polypeptide with mutations which are found in cancer cells such as ovarian cancer cells; however, such polypeptides may be useful in provoking an anti-cancer cell immune response. Thus, according to the present invention, a method is also provided of supplying wild-type Barx2 function to a cell which carries mutant Barx2 alleles. Supplying such a function should suppress neoplastic growth of the recipient cells. The wild-type Barx2 gene or a part of the gene may be introduced into the cell in a vector such that the gene remains extrachromosomal. In such a situation, the gene will be expressed by the cell from the extrachromosomal location. If a gene fragment is introduced and expressed in a cell carrying a mutant Barx2 allele, the gene fragment should encode a part of the Barx2 protein which is required for non-neoplastic growth of the cell. More preferred is the situation where the wild-type Barx2 gene or a part thereof is introduced into the mutant cell in such a way that it recombines with the endogenous mutant Barx2 gene present in the cell. Such recombination requires a double recombination event which results in the correction of the Barx2 gene mutation. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the suitably skilled person. Cells transformed with the wild-type Barx2 gene can be used as model systems to study cancer remission and drug treatments which promote such remission.

As generally discussed above, the Barx2 gene or fragment, where applicable, may be employed in gene therapy methods in order to increase the amount of the expression products of such genes in cancer cells. Such gene therapy is particularly appropriate for use in both cancerous and pre-cancerous cells, in which the level of Barx2 polypeptide is absent or diminished or otherwise changed compared to normal cells. It may also be useful to increase the level of expression of a given Barx2 gene even in those tumour cells in which the mutant gene is expressed at a “normal” level, but the gene product is not fully functional or has an altered function.

Gene therapy may be carried out according to generally accepted methods, for example, as described by Friedman, 1991. Cells from a patient's tumour may be first analyzed by the diagnostic methods described herein, to ascertain the production of Barx2 polypeptide and its physical form (ie what mutations it contains) in the tumour cells. A virus or plasmid vector (see further details below), containing a copy of the Barx2 gene linked to expression control elements and capable of replicating inside the tumour cells, is prepared. Suitable vectors are known, such as disclosed in U.S. Pat. No. 5,252,479 and PCT published application WO 93/07282. The vector is then injected into the patient, either locally at the site of the tumour or systemically (in order to reach any tumour cells that may have metastasized to other sites). If the transfected gene is not permanently incorporated into the genome of each of the targeted tumour cells, the treatment may have to be repeated periodically.

Gene transfer systems known in the art may be useful in the practice of the gene therapy methods of the present invention. These include viral and nonviral transfer methods. A number of viruses have been used as gene transfer vectors, including papovaviruses, eg SV40 (Madzak et al, 1992), adenovirus (Berkner, 1992; Berkner et al, 1988; Gorziglia and Kapikian, 1992; Quantin et al, 1992; Rosenfeld et al, 1992; Wilkinson et al, 1992; Stratford-Perricaudet et al, 1990), vaccinia virus (Moss, 1992), adeno-associated virus (Muzyczka, 1992; Ohi et al, 1990), herpesviruses including HSV and EBV (Margolskee, 1992; Johnson et al, 1992; Fink et al, 1992; Breakfield and Geller, 1987; Freese et al, 1990), and retroviruses of avian (Brandyopadhyay and Temin, 1984; Petropoulos et al., 1992), murine (Miller, 1992; Miller et al, 1985; Sorge et al, 1984; Mann and Baltimore, 1985; Miller et al, 1988), and human origin (Shimada et al, 1991; Helseth et al, 1990; Page et al, 1990; Buchschacher and Panganiban, 1992). To date most human gene therapy protocols have been based on disabled murine retroviruses.

Nonviral gene transfer methods known in the art include chemical techniques such as calcium phosphate coprecipitation (Graham and van der Eb, 1973; Pellicer et al, 1980), mechanical techniques, for example microinjection (Anderson et al, 1980; Gordon et al, 1980; Brinster et al, 1981; Constantini and Lacy, 1981); membrane fusion-mediated transfer via liposomes (Felgner et al, 1987; Wang and Huang, 1989; Kaneda et al, 1989; Stewart et al, 1992; Nabel et al, 1990; Lim et al, 1992); and direct DNA uptake and receptor-mediated DNA transfer (Wolff et al, 1990; Wu et al, 1991; Zenke et al, 1990; Wu et al, 1989b; Wolff et al, 1991; Wagner et al, 1990; Wagner et al, 1991; Cotten et al, 1990; Curiel et al, 1991a; Curiel et al, 1991b). Viral-mediated gene transfer can be combined with direct in vivo gene transfer using liposome delivery, allowing one to direct the viral vectors to the tumour cells and not into the surrounding nondividing cells. Alternatively, the retroviral vector producer cell line can be injected into tumours (Culver et al, 1992). Injection of producer cells would then provide a continuous source of vector particles. This technique has been approved for use in humans with inoperable brain tumours.

Other suitable systems include the retroviral-adenoviral hybrid system described by Feng et al (1997) Nature Biotechnology 15, 866-870, or viral systems with targeting ligands such as suitable single chain Fv fragments.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

Liposome/DNA complexes have been shown to be capable of mediating direct in vivo gene transfer. While in standard liposome preparations the gene transfer process is nonspecific, localized in vivo uptake and expression have been reported in tumour deposits, for example, following direct in situ administration (Nabel, 1992).

Gene transfer techniques which target DNA directly to ovarian tissue, eg epithelial cells of the ovaries, is preferred. Receptor-mediated gene transfer, for example, is accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, coinfection with adenovirus can be included to disrupt endosome function.

In the case where replacement gene therapy using a functionally wild-type Barx2 is used, it may be useful to monitor the treatment by detecting the presence of Barx2 mRNA or polypeptide, or functional Barx2, at various sites in the body, including the targeted tumour, sites of metastasis, blood serum, and bodily secretions/excretions, for example urine.

A still further aspect of the invention provides a vector for expression in a mammalian cell, preferably in a human cell, of the Barx2 polypeptide or a functional fragment or variant or fusion thereof. Typically, the functional fragment or variant of the Barx2 polypeptide has the tumour-suppressing activities of wild-type Barx2.

Preferably, the vector is one which can replicate in a human cell. Preferably, the vector is one which has been described in more detail above in connection with the gene therapy aspects of the invention.

A further aspect of the invention provides a method of treating cancer comprising the step of administering to the patient an effective amount of Barx2 polypeptide or a fragment or variant or fusion thereof to ameliorate the cancer.

Peptides which have Barx2 activity can be supplied to cells which carry mutant or missing Barx2 alleles. The sequence of the Barx2 protein is disclosed in FIGS. 1a and 1b. Protein can be produced by expression of the cDNA sequence in bacteria, for example, using known expression vectors. Alternatively, Barx2 polypeptide can be extracted from Barx2-producing mammalian cells. In addition, the techniques of synthetic chemistry can be employed to synthesize Barx2 protein. Any of such techniques can provide the preparation of the present invention which comprises the Barx2 protein. The preparation is substantially free of other human proteins. This is most readily accomplished by synthesis in a microorganism or in vitro.

The Barx2 gene or cDNA can be expressed by any suitable method. Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells. An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps)

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

A still further aspect of the invention provides a method of treating cancer comprising the step of administering to the patient an effective amount of a compound which inhibits the function of a mutant Barx2 polypeptide found in a tumour cell, or which upregulates expression of wild-type Barx2 polypeptide.

Suitable compounds for use in this method of the invention include antibodies or fragments or variants thereof which inhibit the activity of the mutant Barx2, or antisense molecules which inhibit the expression of the mutant Barx2.

Alternatively, suitable compounds may be obtained by screening. Screening compounds by using the Barx2 polypeptide or binding fragment thereof in any of a variety of drug screening techniques may be used.

The Barx2 polypeptide or fragment or a mutant thereof found in a tumour cell employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, for the formation of complexes between a Barx2 polypeptide or fragment and the agent being tested, or examine the degree to which the formation of a complex between a Barx2 polypeptide, or fragment and a known ligand is interfered with by the agent being tested.

Thus, the present invention provides methods of screening for drugs comprising contacting such an agent with a Barx2 polypeptide or fragment thereof or a mutant thereof found in a tumour cell and assaying (i) for the presence of a complex between the agent and the Barx2 polypeptide or fragment or mutant, or (ii) for the presence of a complex between the Barx2 polypeptide or fragment or mutant and a ligand, by methods well known in the art. In such competitive binding assays the Barx2 polypeptide or fragment or mutant is typically labeled. Free Barx2 polypeptide or fragment or mutant is separated from that present in a protein:protein complex and the amount of free (ie uncomplexed) label is a measure of the binding of the agent being tested to Barx2 or its interference with Barx2:ligand binding, respectively.

Drugs which are able to correct mutant Barx2 function (so that the wild-type function is restored) are believed to be useful. Similarly, drugs which promote expression of wild-type Barx2 are believed to be useful.

Another technique for drug screening provides high throughput screening for compounds having suitable binding affinity to the Barx2 polypeptides and is described in detail in Geysen, PCT published application WO 84/03564, published on Sep. 13, 1984. Briefly stated, large numbers of different small peptide test compounds are synthesized on a solid substrate, such as plastic pins or some other surface. The peptide test compounds are reacted with Barx2 polypeptide and washed. Bound Barx2 polypeptide is then detected by methods well known in the art.

Purified Barx2 can be coated directly onto plates for use in the aforementioned drug screening techniques. However, non-neutralizing antibodies to the polypeptide can be used to capture antibodies to immobilize the Barx2 polypeptide on the solid phase.

This invention also contemplates the use of competitive drug screening assays in which neutralizing antibodies capable of specifically binding the Barx2 polypeptide compete with a test compound for binding to the Barx2 polypeptide or fragments thereof. In this manner, the antibodies can be used to detect the presence of any peptide which shares one or more antigenic determinants of the Barx2 polypeptide.

A further technique for drug screening involves the use of host eukaryotic cell lines or cells (such as described above) which have a mutant Barx2 gene. These host cell lines or cells are defective at the Barx2 polypeptide level. The host cell lines or cells are grown in the presence of drug compound. The rate of growth of the host cells is measured to determine if the compound is capable of regulating the growth of Barx2 defective cells.

Screens may also be derived which make use of the Barx2 promoter sequence operatively linked to a reporter gene. Compounds which selectively increase the expression of the reporter gene may be usefully selected.

Additionally or alternatively, rational drug design may be used. The goal of rational drug design is to produce structural analogs of biologically active polypeptides of interest or of small molecules with which they interact (eg agonists, antagonists, inhibitors) in order to fashion drugs which are, for example, more active or stable forms of the polypeptide, or which, eg enhance or interfere with the function of a polypeptide in vivo. See, eg Hodgson, 1991. In one approach, one first determines the three-dimensional structure of a protein of interest (eg Barx2 polypeptide) or, for example, of the Barx2 ligand complex, by x-ray crystallography, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al, 1990). In addition, peptides (eg Barx2 polypeptide) are analyzed by an alanine scan (Wells, 1991). In this technique; an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacophore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Thus, one may design drugs which have, for example, improved Barx2 polypeptide activity or stability or which act as inhibitors, agonists, antagonists, etc of Barx2 polypeptide activity. By virtue of the availability of cloned Barx2 sequences, sufficient amounts of the Barx2 polypeptide may be made available to perform such analytical studies as x-ray crystallography. In addition, the knowledge of the Barx2 protein sequence provided herein will guide those employing computer modeling techniques in place of, or in addition to x-ray crystallography.

Cells and animals which carry a mutant Barx2 allele can be used as model systems to study and test for substances which have potential as therapeutic agents. The cells are typically cultured epithelial cells. These may be isolated from individuals with Barx2 mutations, either somatic or germline. Alternatively, the cell line can be engineered to carry the mutation in the Barx2 allele, using methods well known in the art. After a test substance is applied to the cells, the neoplastically transformed phenotype of the cell is determined. Any trait of neoplastically transformed cells can be assessed, including anchorage-independent growth, tumourigenicity in nude mice, invasiveness of cells, and growth factor dependence. Assays for each of these traits are known in the art.

Animals for testing therapeutic agents can be selected after mutagenesis of whole animals or after treatment of germline cells or zygotes. Such treatments include insertion of mutant Barx2 alleles, usually from a second animal species, as well as insertion of disrupted homologous genes. Alternatively, the endogenous Barx2 gene(s) of the animals may be disrupted by insertion or deletion mutation or other genetic alterations using conventional techniques (Capecchi, 1989; Valancius and Smithies, 1991; Hasty et al, 1991; Shinkai et al, 1992; Mombaerts et al, 1992; Philpott et al, 1992; Snouwaert et al, 1992; Donehower et al, 1992). After test substances have been administered to the animals, the growth of tumours must be assessed. If the test substance prevents or suppresses the growth of tumours, then the test substance is a candidate therapeutic agent for the treatment of the cancers identified herein. These animal models provide an extremely important testing vehicle for potential therapeutic products.

Active Barx2 molecules can be introduced into cells by microinjection or by use of liposomes, for example. Alternatively, some active molecules may be taken up by cells, actively or by diffusion. Extracellular application of the Barx2 gene product may be sufficient to affect tumour growth. Supply of molecules with Barx2 activity should lead to partial reversal of the neoplastic state. Other molecules with Barx2 activity (for example, peptides, drugs or organic compounds) may also be used to effect such a reversal. Modified polypeptides having substantially similar function are also used for peptide therapy.

Further aspects of the invention provide pharmaceutical composition comprising a gene therapy vector including a nucleic acid which encodes the Barx2 polypeptide or a functional variant or portion or fusion thereof and pharmaceutically acceptable carrier; a pharmaceutical composition comprising a gene therapy vector including a nucleic acid which selectively hybridises to the Barx2 gene, or a mutant allele thereof, or a Barx2 cDNA, or a mutant allele thereof, and a pharmaceutically acceptable carrier; a pharmaceutical composition comprising Barx2 polypeptide or a fragment or variant or fusion thereof, and a pharmaceutically acceptable carrier.

Suitable gene therapy vectors are described above. Suitable Barx2 polypeptides are described above.

By “pharmaceutically acceptable” is included that the formulation is sterile and pyrogen free. Suitable pharmaceutical carriers are well known in the art of pharmacy.

The present invention will now be described in more detail with reference to the following Examples and Figures wherein

FIG. 1a shows the cDNA sequence derived from human Barx2 mRNA and the translated product. The position of two missense mutations is marked. A human Barx2 cDNA sequence is also shown in GenBank Accession No NM003658.2.

FIG. 1b shows the cDNA sequence derived from human Barx2 mRNA and the translated product as described in GenBank Accession No AF 031924.

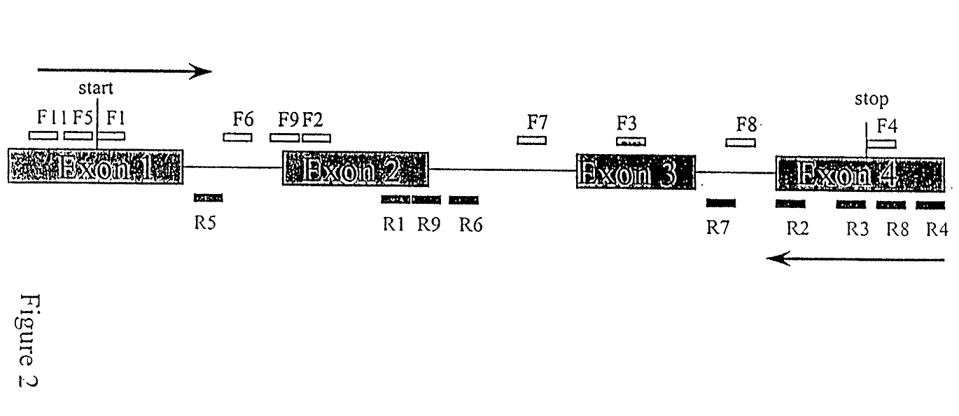

FIG. 2 shows a diagrammatic representation of the human Barx2 gene with PCR primers used in various studies marked.

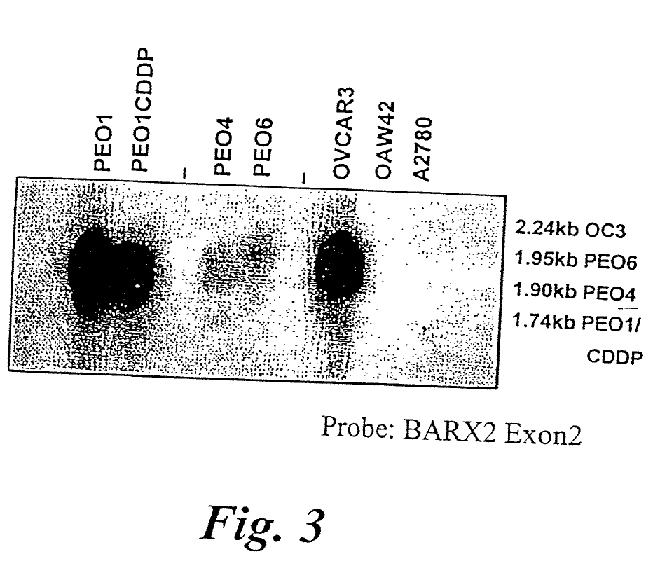

FIG. 3 shows a northern blot which indicates differential expression of Barx2 in ovarian cancer cell lines. Exon 2 of Barx2 was used as a probe.

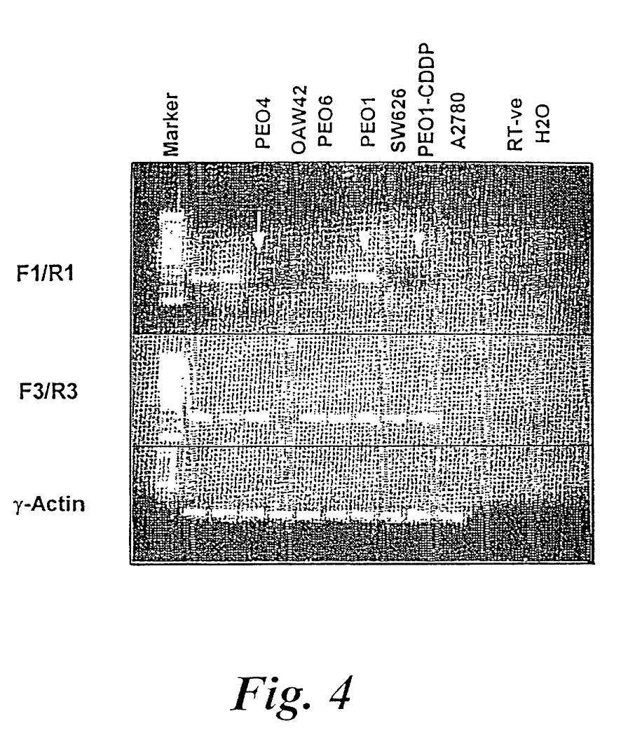

FIG. 4 shows the expression of Barx2 in various ovarian cell lines. Primers F1/R1 and F3/R3 were used in RT-PCR experiments. Transfection with Barx2 leads to a decrease in platinum resistance.

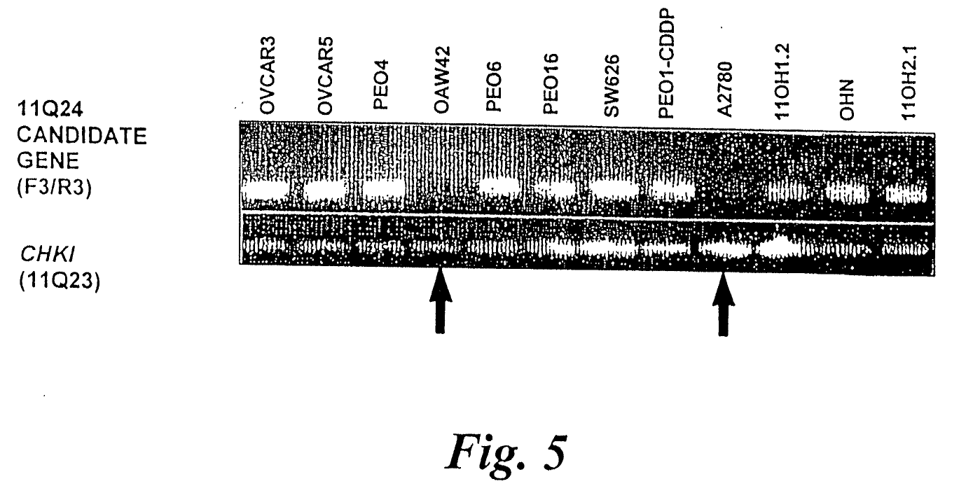

FIG. 5 shows that Barx2 shows essentially undetectable expression by RT-PC is two out of seven ovarian cancer cell lines (OAW 42 and A2780). This represents a similar experiment to FIG. 3. This experiment shows RT-PCR of the 3′ end of the BARX2 gene in ovarian cancer cell lines. BARX2 is expressed in all the ovarian cancer cell lines shown. BARX2 expression is essentially absent in OAW42 and A2780 (confirms northern blot). 11OH2.1 is a microcell hybrid containing a normal copy of chromosome 11 transferred into OVCAR3.

CHK1 is a cell cycle regulatory gene located on 11q23 nearby. Expression of this gene is ubiquitous in all lines tested so far.

FIG. 6 shows genomic PCRs of OVCAR3 cell line transfected with Barx2 cDNA.

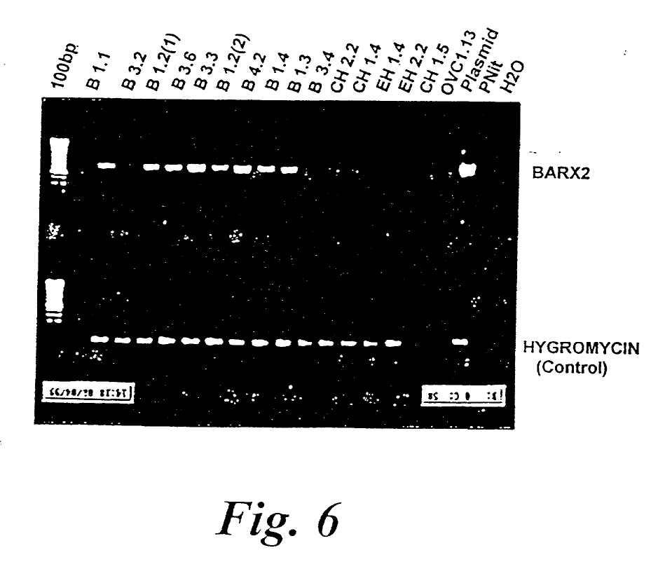

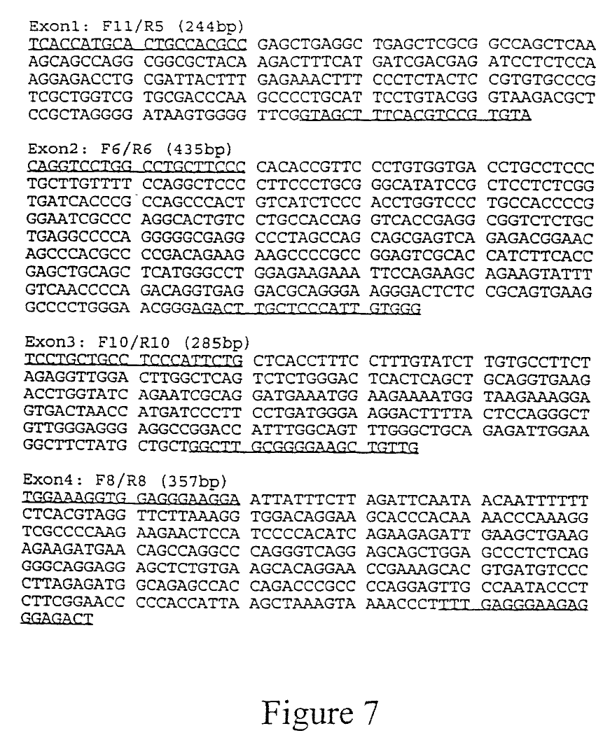

FIG. 7 shows the DNA sequence of the genomic Barx2 PCR products.

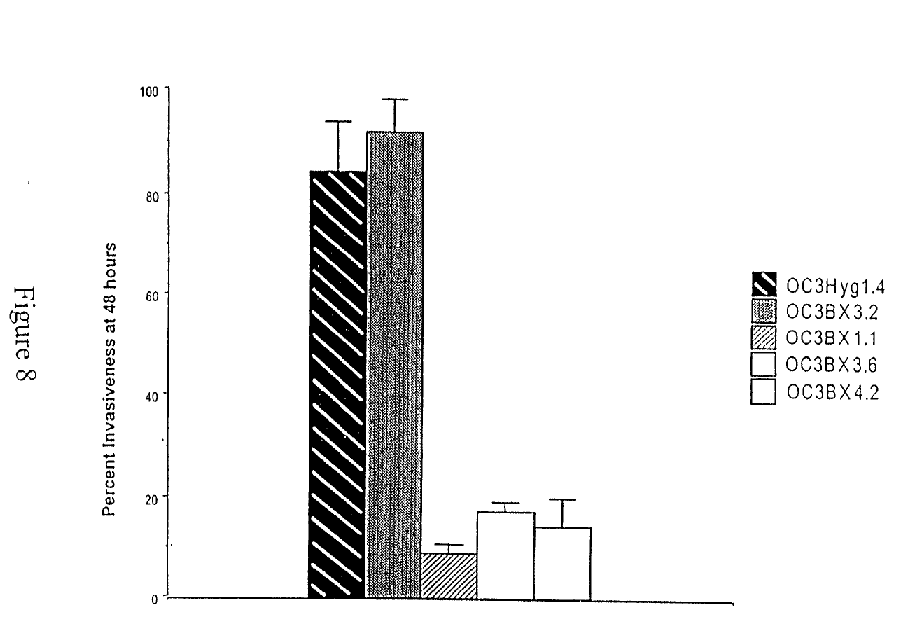

FIG. 8 shows data from “invasion Bx”. This assay is based on the principle that the basement membrane plays an important part as a barrier against tumour cell invasion.

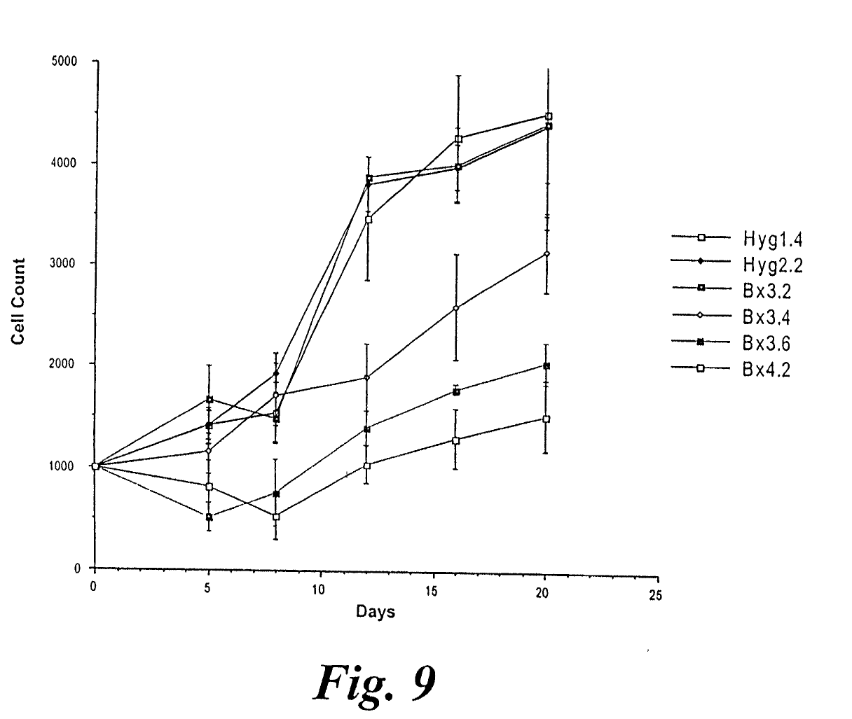

FIG. 9 shows OVCAR3 cell line transfections with Hyg and Barx2.

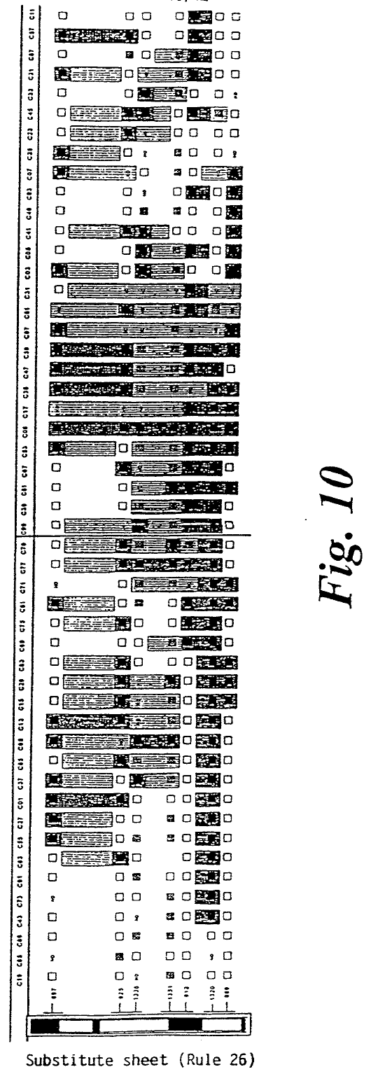

FIG. 10 shows schematic representation of regions of LOH in all of the tumor samples. Case numbers for patients' blood tumor pairs are shown at the top. Microsatellite loci, used to detect LOH are at the left of the diagram, and their approximate position is indicated on the chromosome 11q idiogram. Shaded areas correspond to regions of LOH. Dark shadowing, a region of secure LOH; Light shadowing, a region of uncertainty; Unshadowed, heterozygosity. Light boxes, loci maintaining heterozygosity without LOH; checked boxes, uninformative (homozygous) loci; dark boxes, LOH; ND, not determinable.

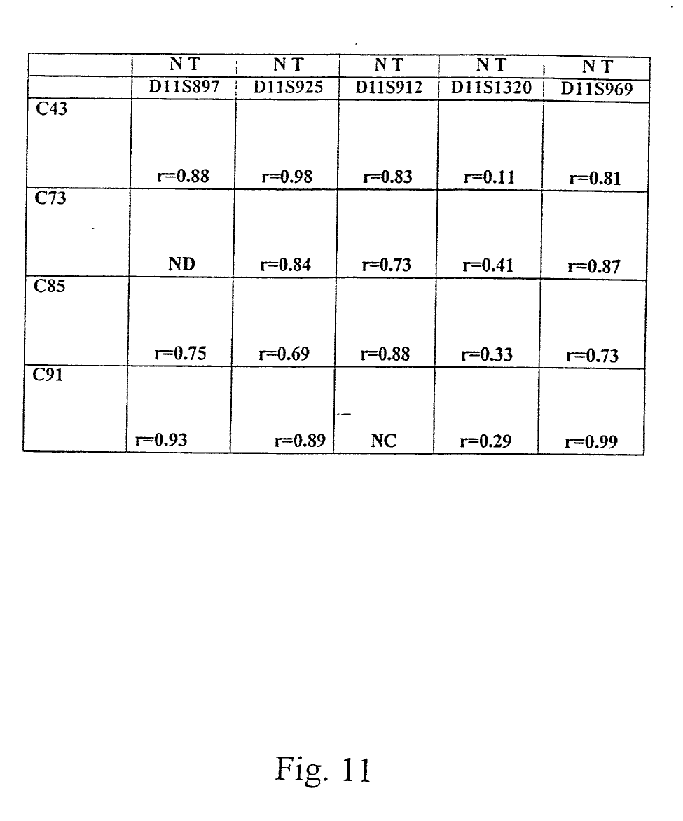

FIG. 11 shows primary LOH data from four cases critical to the definition of the 11q23.3-q24.3 locus. N, normal DNA; T, tumor DNA shown at top from patients C43, C73, C85 and C91 (left). Microsatellite s loci are shown from centromeric (top left) to telomeric (top right). Arrows, alleles showing LOH (allele imbalance). Densitometric ratios of allele intensity were calculated (shown at bottom) and values between 0.0 and 0.7 are taken to indicate LOH. U, uninformative (homozygous).

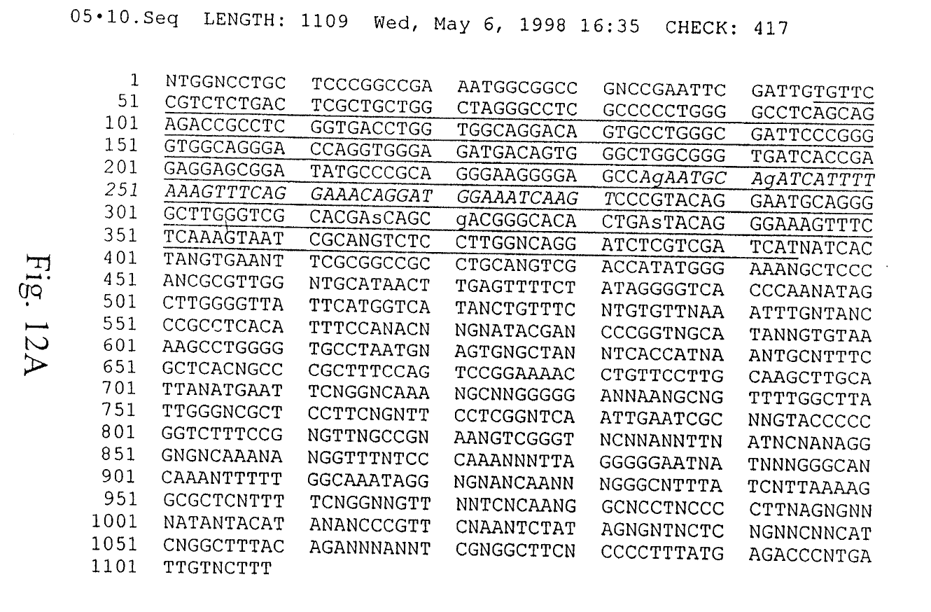

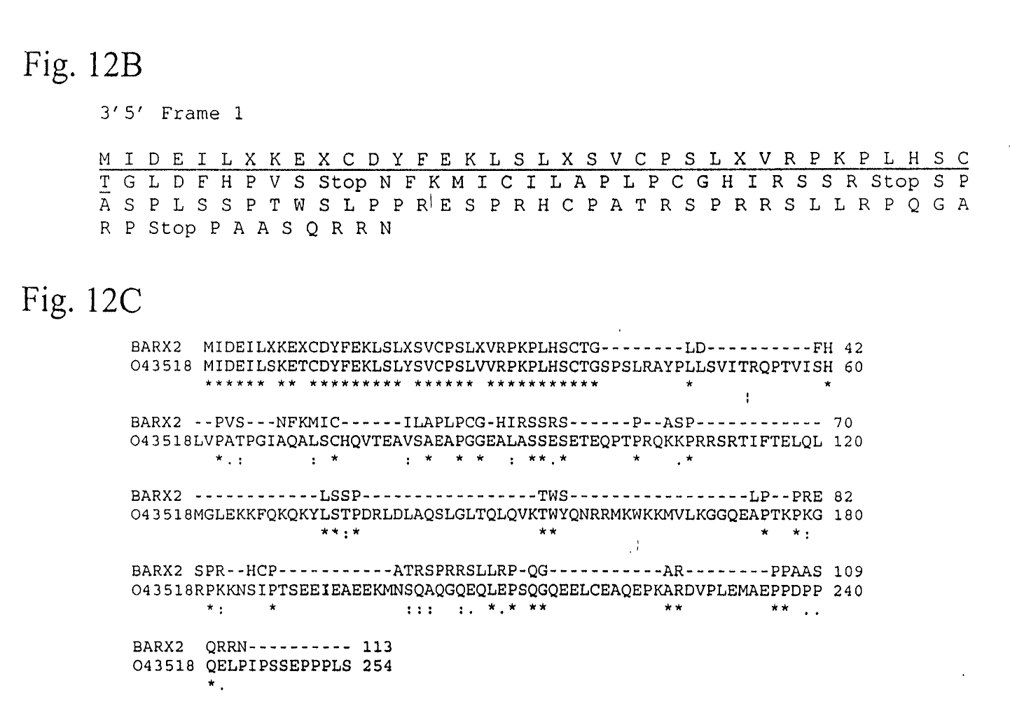

FIG. 12 shows the mutation in the Barx2 gene.

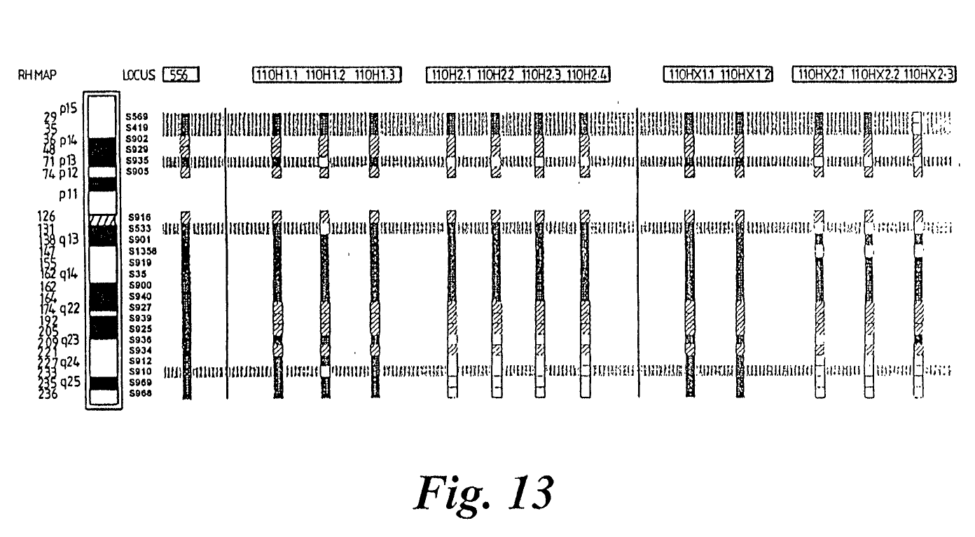

FIG. 13 shows schematic representation of the analysis of transferred alleles MCH 556.1.5's donor chromosome 11 to OVCAR3 sublines OH1 and OHX. Names of cell lines/microcell hybrids are shown at top. Microsatellite loci used for this analysis are shown at left of diagram. Their approximate position is indicated with respect to the chromosome 11 idiogram. The radiation hybrid map position of each of these polymorphic microsatellites is shown at extreme left. Horizontal shadowed bars show the regions where disruption of the donated chromosome are associated with functional reversion. Dark hatched boxes represent evidence of chromosome transfer at that locus. Diagonal hatched boxes represent non-informative (homozygous) markers at that locus. Open boxes represent evidence that the donor allele was not transferred at that locus.

FIG. 14 shows i) OHN control, ii) MHC 11OH2.4 (transfer of Chr 11 lacking q22-qter), iii) MHC 11OH1.2 (revertant hybrid with small deletion of 11q24), iv) MHC 11OH1.3 (transfer of whole Chr 11), A) Morphology of control cell line and microcell hybrids growing on tissue culture plastic showing tight packing and lack of spreading of MHC11OH1.3 compared with control, hybrids with either a small (MHC11OH1.2) or a large (MHC11OH2.1) deletion of 11q, all of which look similar. B) Morphology of control cell line and microcell hybrids after 24 hour attachment to laminin coated tissue culture plastic showing lack of spreading of MHC11OH1.3, and normal spreading of control, hybrids with either a small (MHC11OH1.2) or a large (MHC11OH2.1) deletion of 11q, all of which look similar.

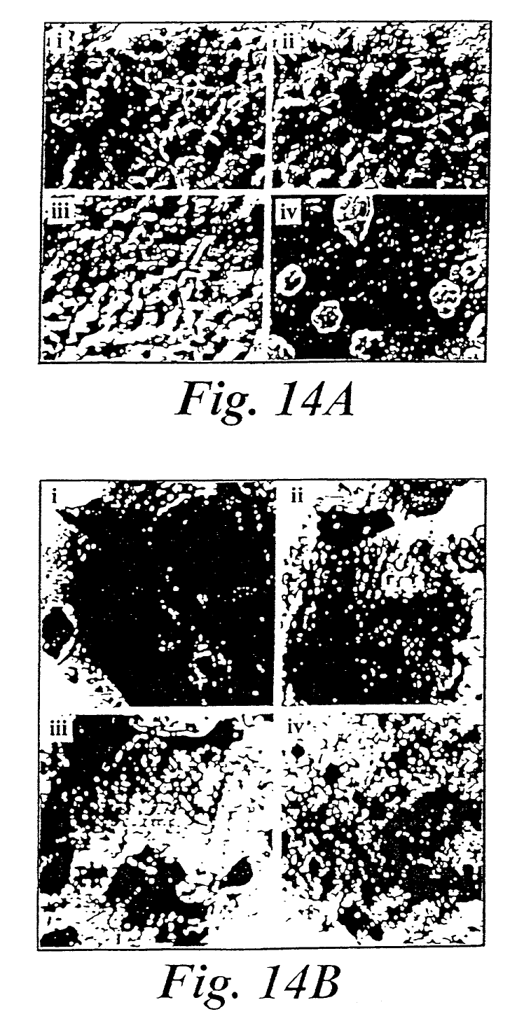

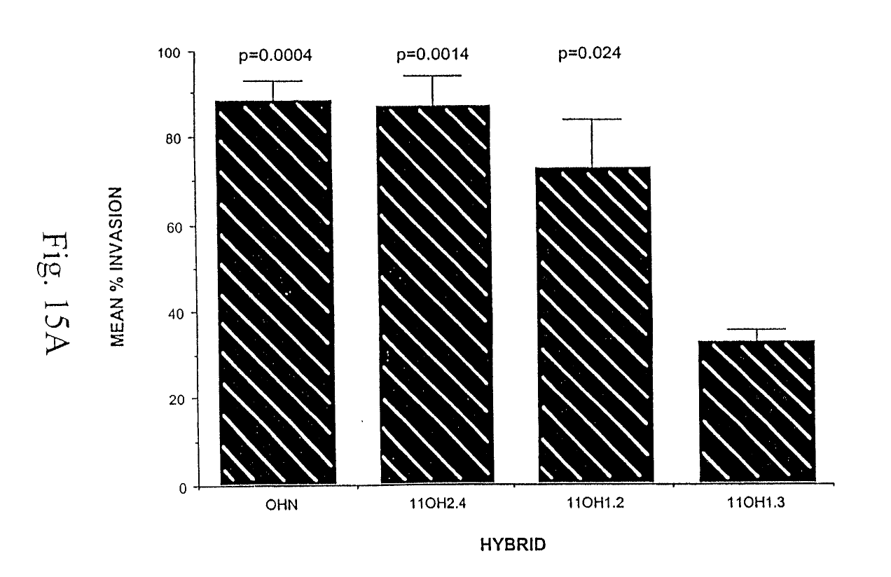

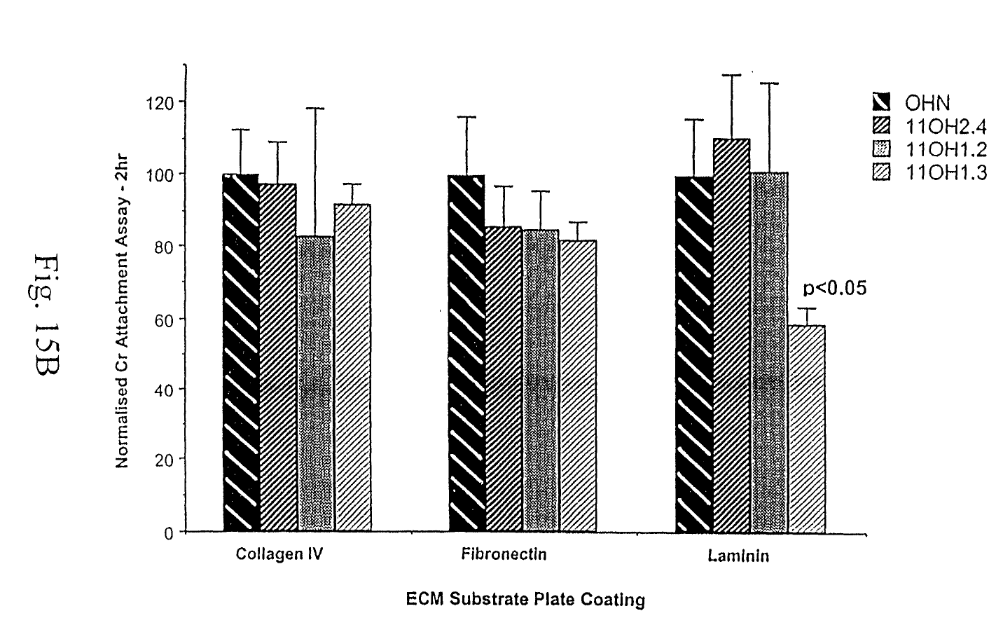

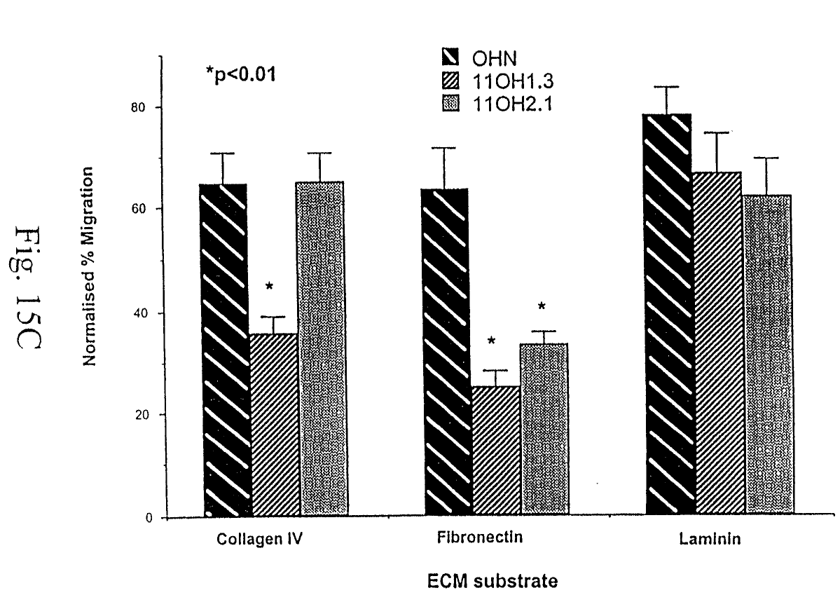

FIG. 15 shows a) 48 hour matrigel invasion assay with direct comparison of control with MHC 11OH1.3 (whole chr 11 transfer), MHC 11OH2.1 (del(11q22-qter)), and MHC 11OH1.2 (small deletion of 11q24). Samples are in quadruplicate and MTT derived quantitation is normalised for control cell line OHN. Error bars represent standard deviation. P value relates to significance of the difference between 11OH1.3 and the other cell line by Dunn's multiple comparison test. b) 2 hour radioactive chromium attachment assay comparing control with microcell hybrids. Attachment values are normalised for control OHN. Hybrid clones are expressed as percent attachment relative to OHN. A typical experiment with samples in quadruplicate. Error bars signify standard deviation. P value relates to Tukey-Kramer multiple comparison test. 11OH1.3 is significantly different from the others. c) Transwell migration assay comparing control and two hybrids with and without the transferred chr 11q22-qter for three ECM components. MTT assay derived quantitation of migration is normalised relative to OHN's migration. A typical experiment is shown. Mean of quadruplicate samples with standard deviations. P value relates to Dunn's multiple comparison test. 11OH1.3 significantly inhibited for migration towards a collagen IV haptotactic signal compared with the others.

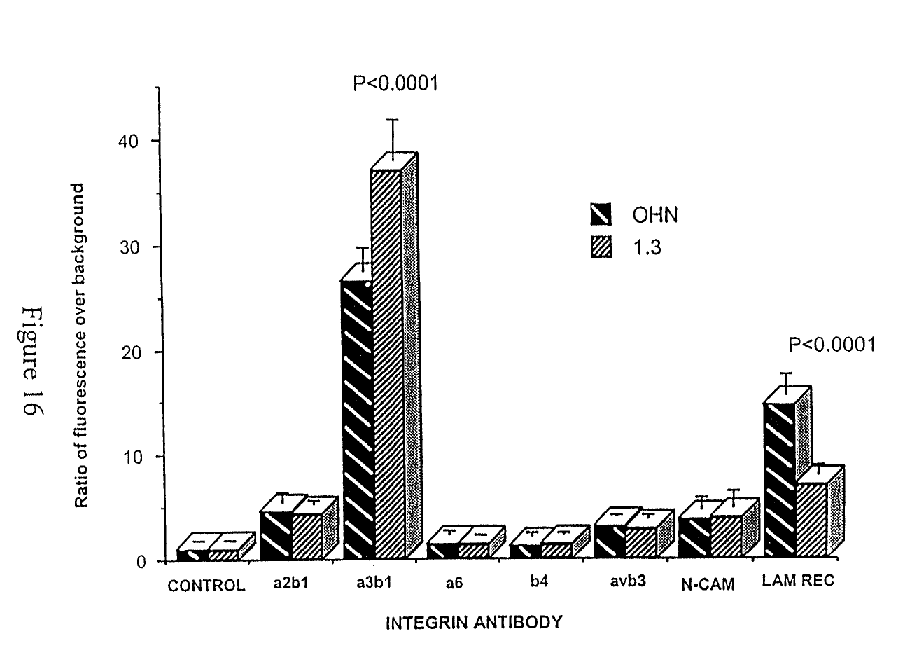

FIG. 16 shows flow-cytometric expression of integrin and non-integrin laminin receptor in OH1, OHX and derived MHCs. In a comparison of control cell line OHN with MHC 11OH1.3 (whole 11 transfer) for integrin expression, a significant decrease of laminin receptor and increase of alpha 3 beta 1 integrin is seen (Mann-Whitney U test p<0.0001).

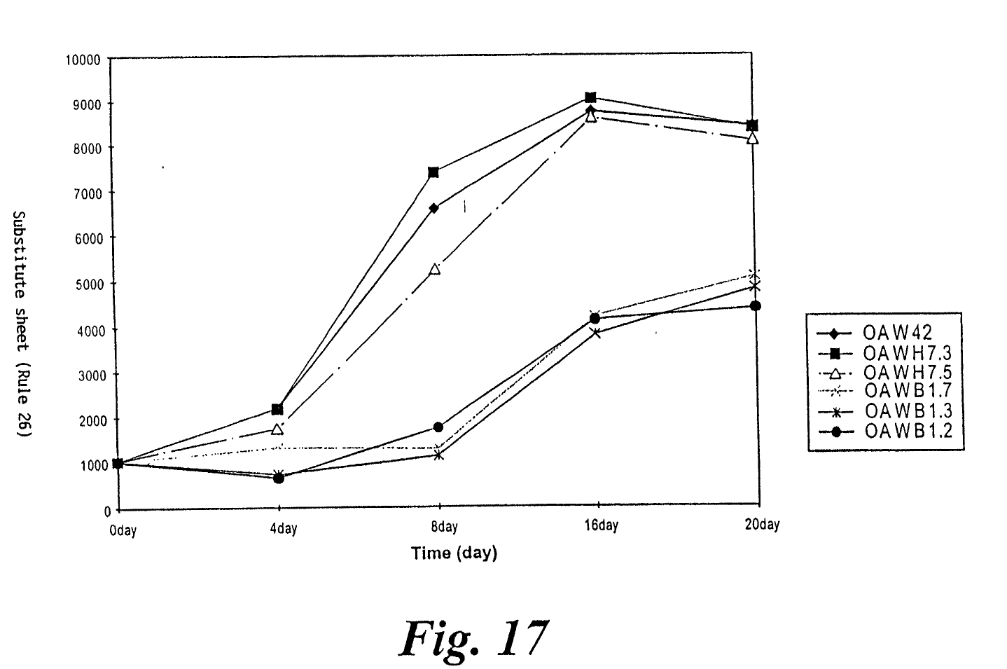

FIGS. 17 to 19 show that overexpression of Barx2 by transfection of pBabeBarx2 suppresses the growth of certain ovarian cancer cell lines.

FIG. 17: 10³ cells plated. OAW42 parent cell line; OAWH7.3 and OAWH7.5 pBabeHygro empty vector transfected controls; OAWB1.2, 1.3 and 1.7 are 3 pBabeBarx2 transfectants.

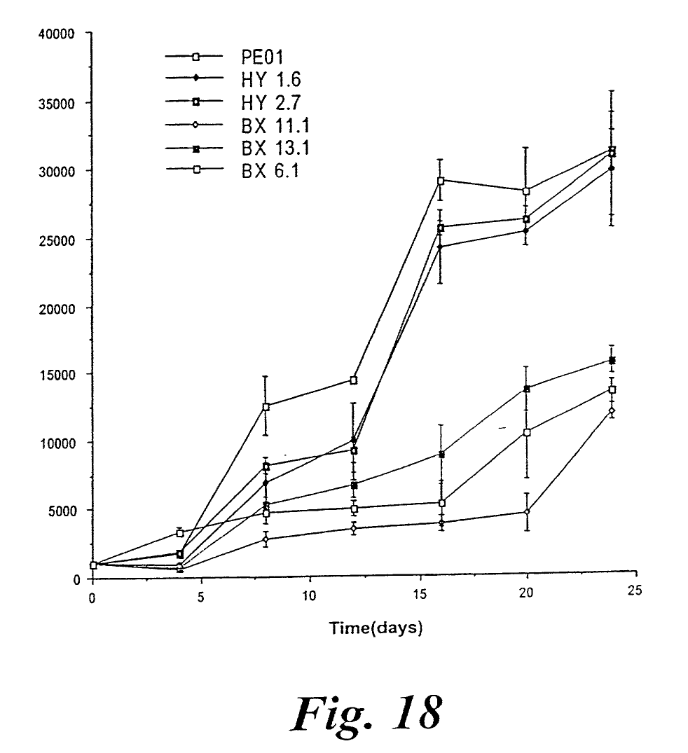

FIG. 18: 10³ cells plated. PEO1 parent cell line.Hy1.6 and Hy2.7 pBabeHygro empty vector transfected controls; BX11.1, 13.1 and 6.1 are 3 pBabeBarx2 transfectants.

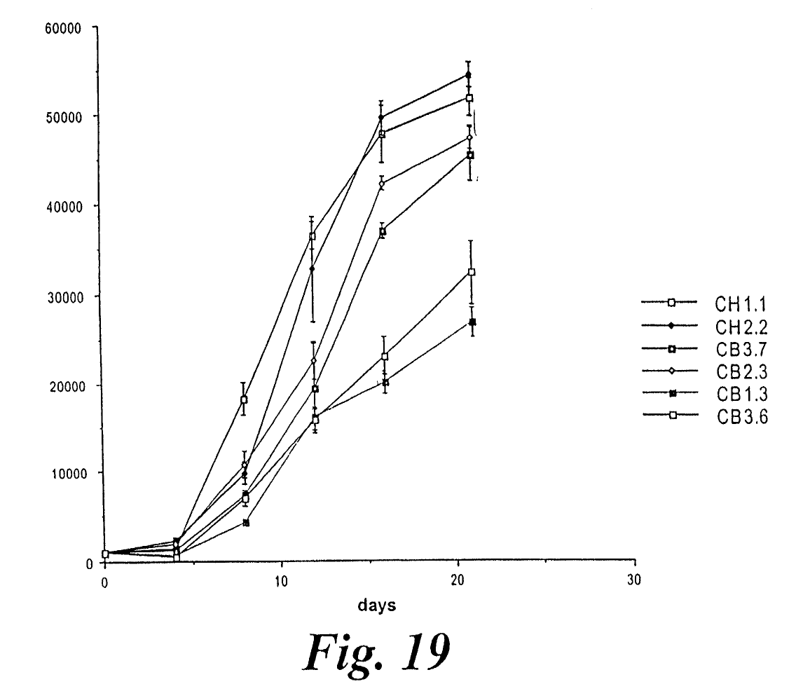

FIG. 19: 10³ cells plated. CH1.1 and CH2.2 are PEO1-CDDP pBabeHygro empty vector transfected controls. CB2.3 and CB3.7 are pBabeBarx2 transfectants exhibiting low copy number plasmid suggesting most cells have lost the Barx2 transfected cDNA. 1.3 and 3.6. CB1.3 and CB3.6 are pBabeBarx2 transfectants exhibiting high copy number plasmid.

FIG. 20 shows that Barx2 transfected clonal lines were generated by transfecting pBabeBarx2, and lines expressing non-endogenous Barx2 were obtained.

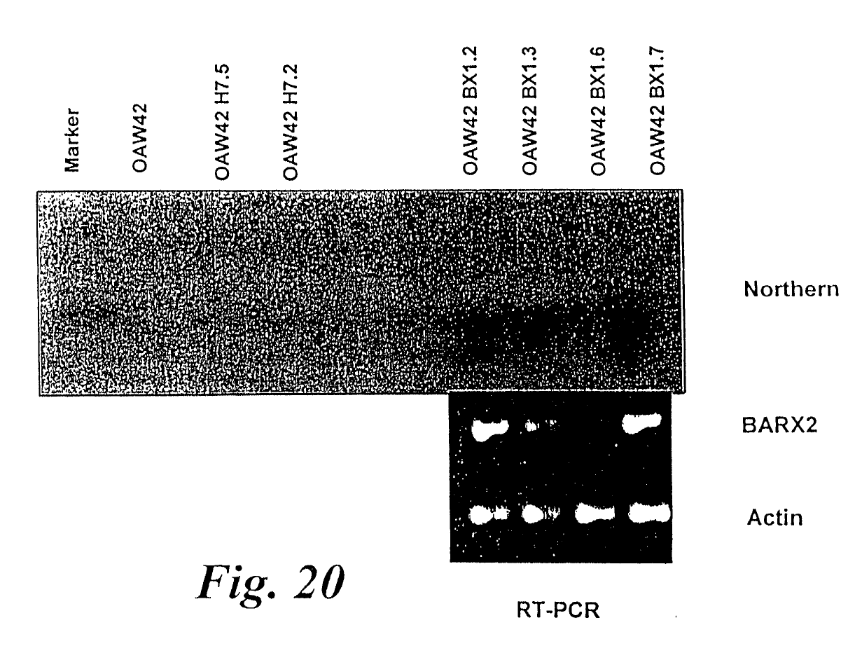

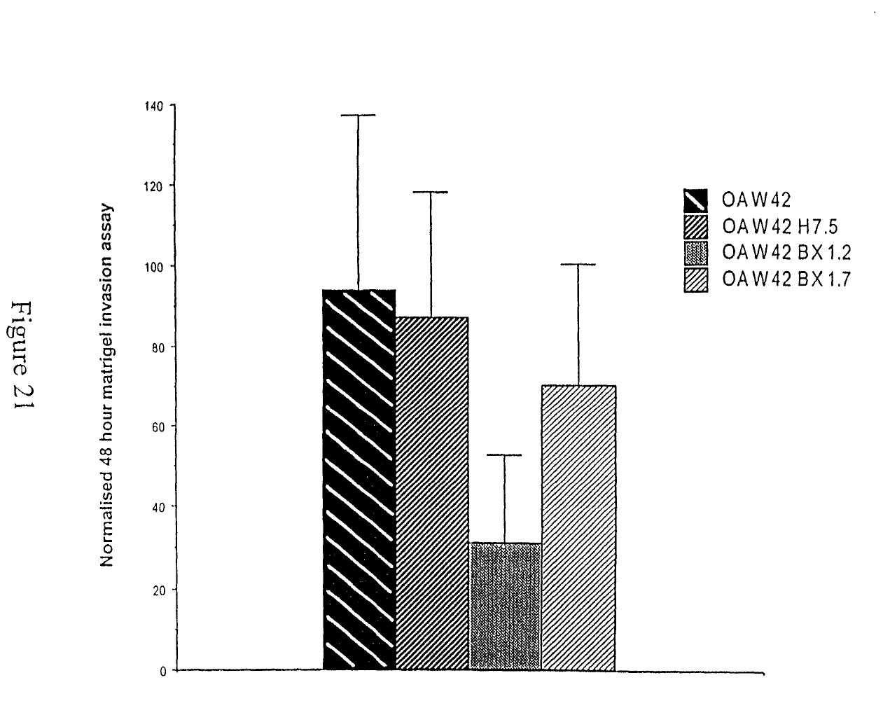

FIG. 21 shows that OAW42 transfected with Barx2 showed suppression of matrigel invasion.

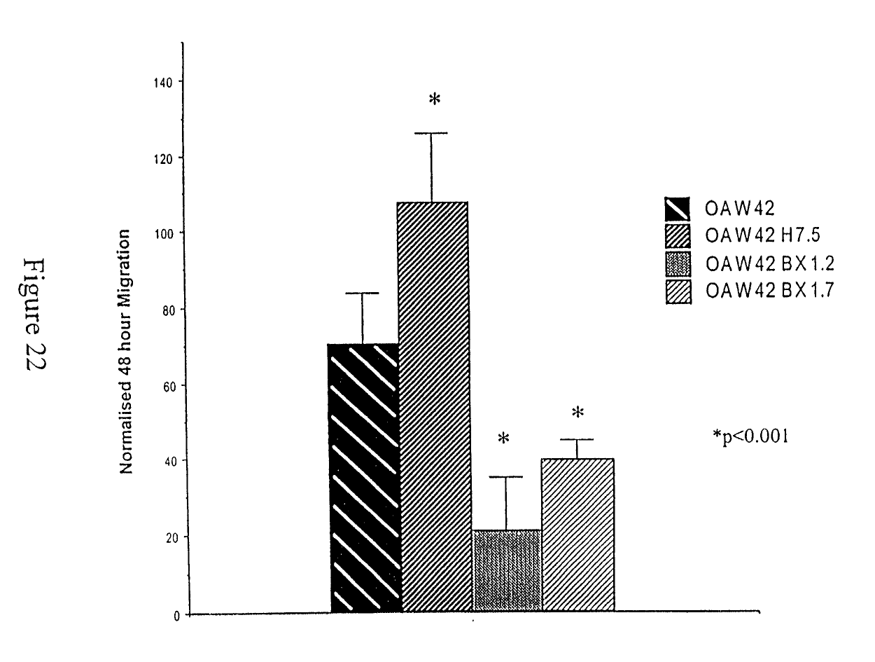

FIG. 22 shows that Barx2 overexpression in OAW42 suppressed cellular migration in response to a collagen haptotactic signal.

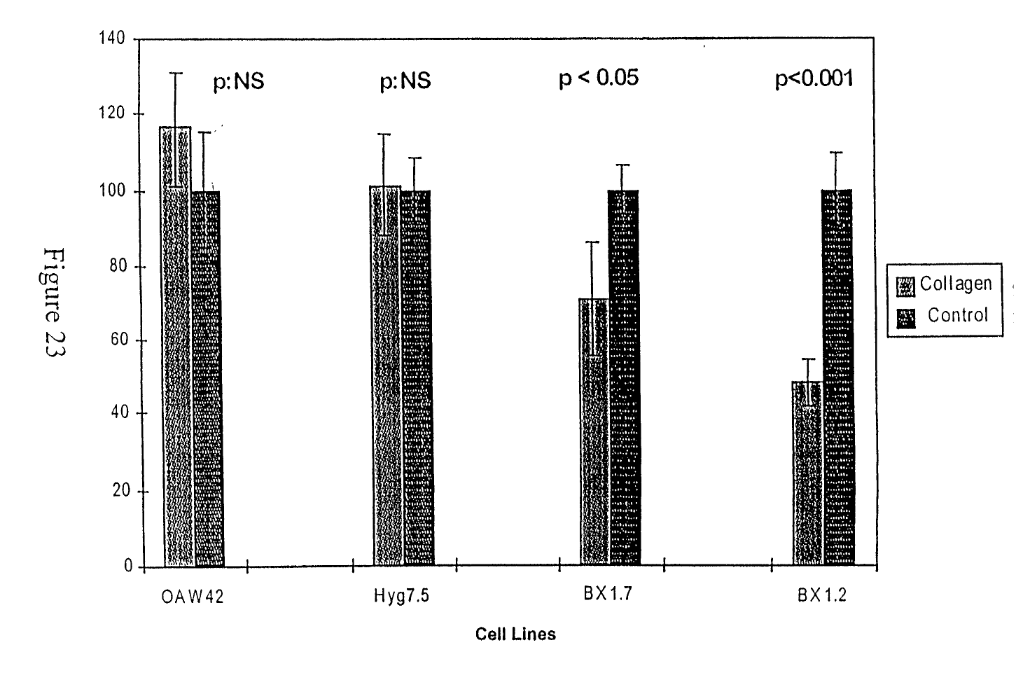

FIG. 23 shows that Barx2 overexpression in OAW42 resulted in suppression of cellular adhesion to collagen coated tissue culture plastic.

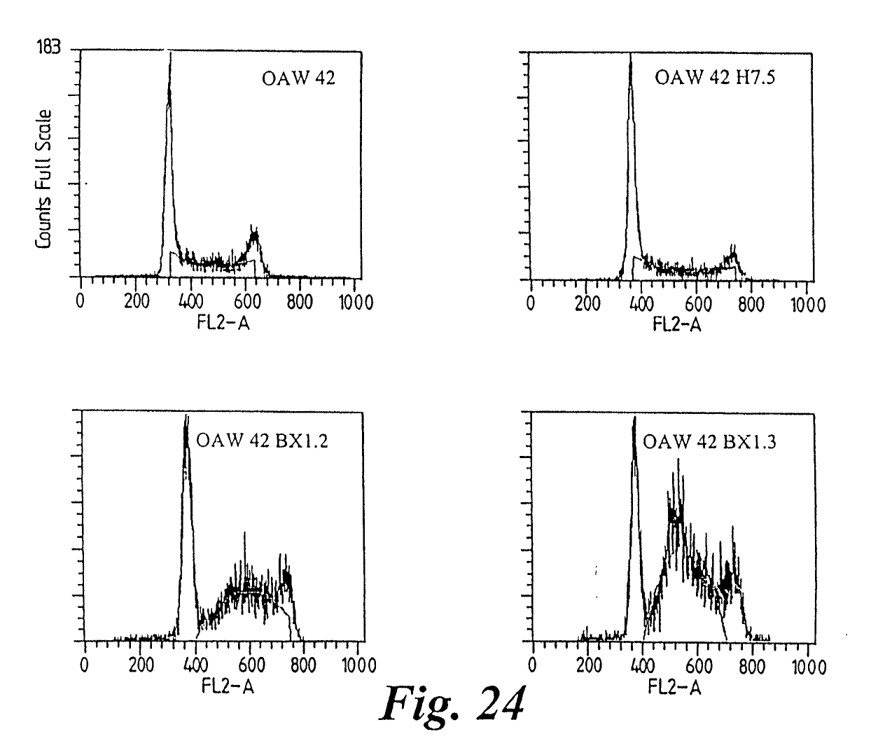

FIG. 24 shows cell cycle analysis of tansfectants reveals that Barx2 overexpression induces late G1/early S phase block.

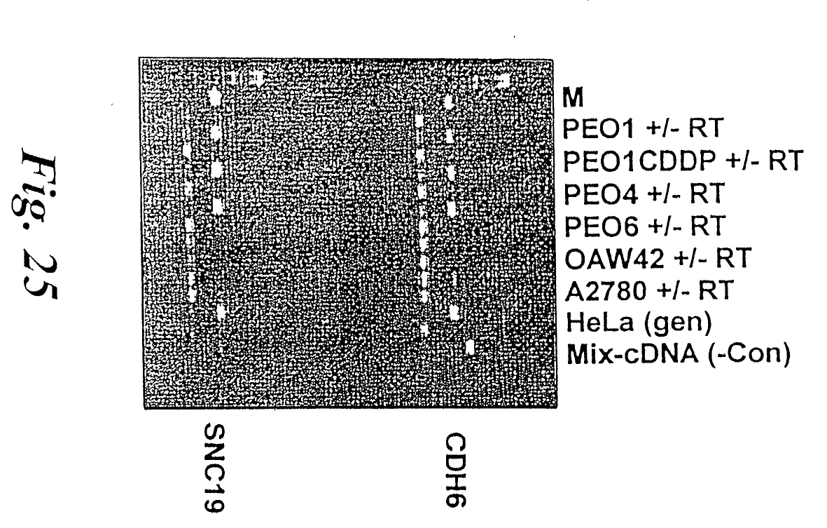

FIG. 25 shows that cadhesin-6 is not expressed in OAW42.

FIG. 26 shows that in BX1.2 and BX1.7 Barx2 expression is directly correlated with k-cadherin expression (CDH6) by RT-PCR.

FIG. 27 shows the effect of transfection of a dominant negative mutant p53 of A2780 (a2780mpa53) on Barx2 and CDH expression.

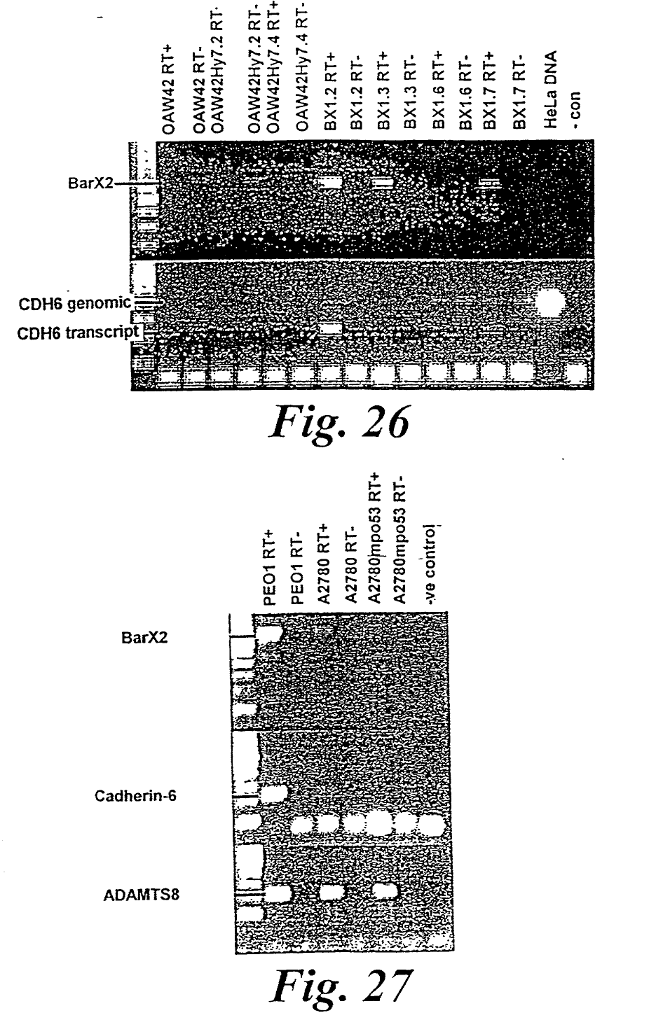

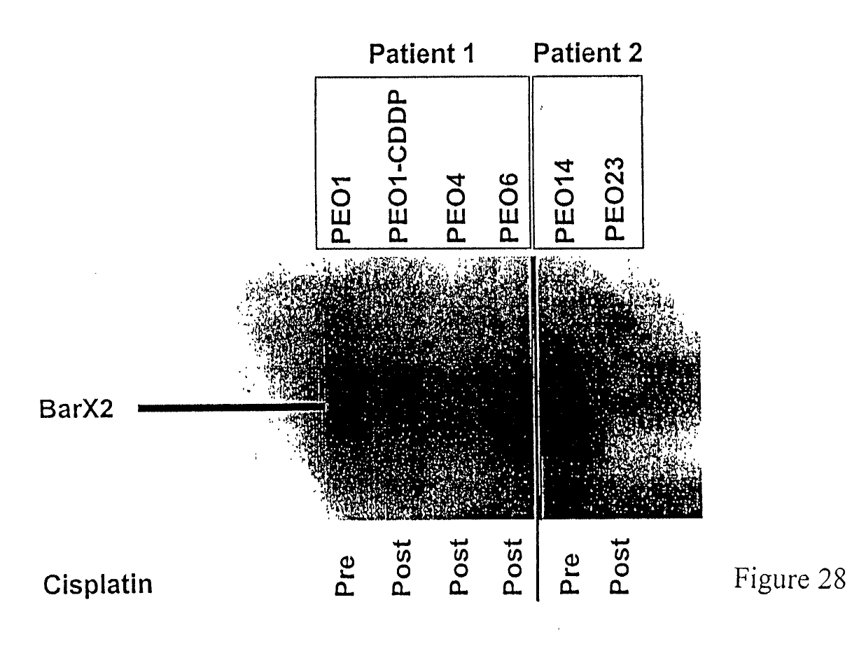

FIG. 28 shows down regulation of Barx2 in platinum resistant cells.

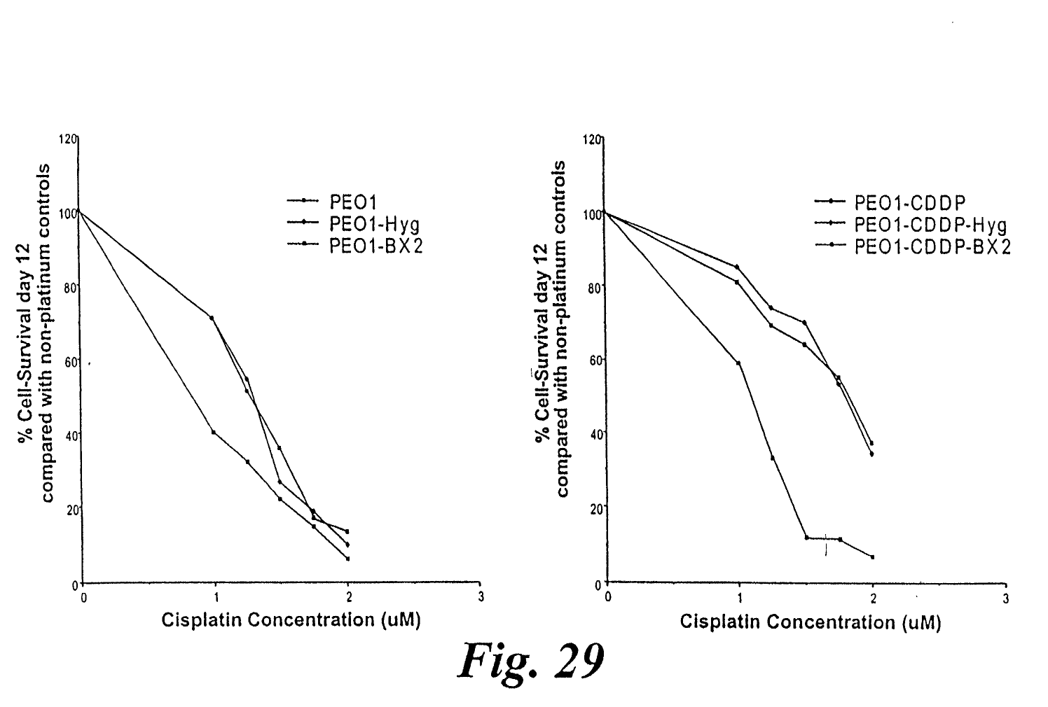

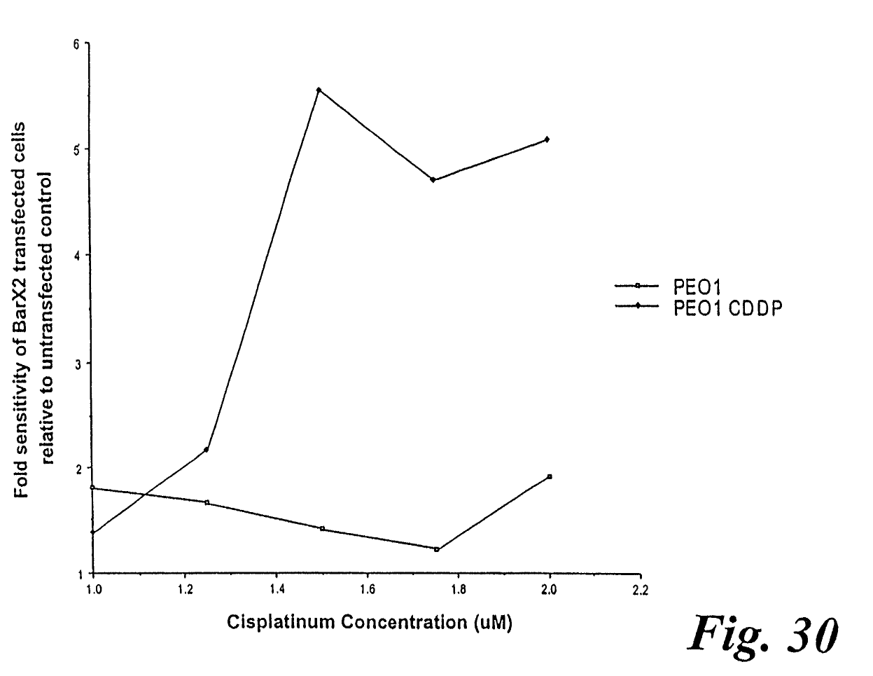

FIG. 29 and 30 show that introduction of Barx2 completely abrogates acquired platinum resistance in PEO1 cell line.

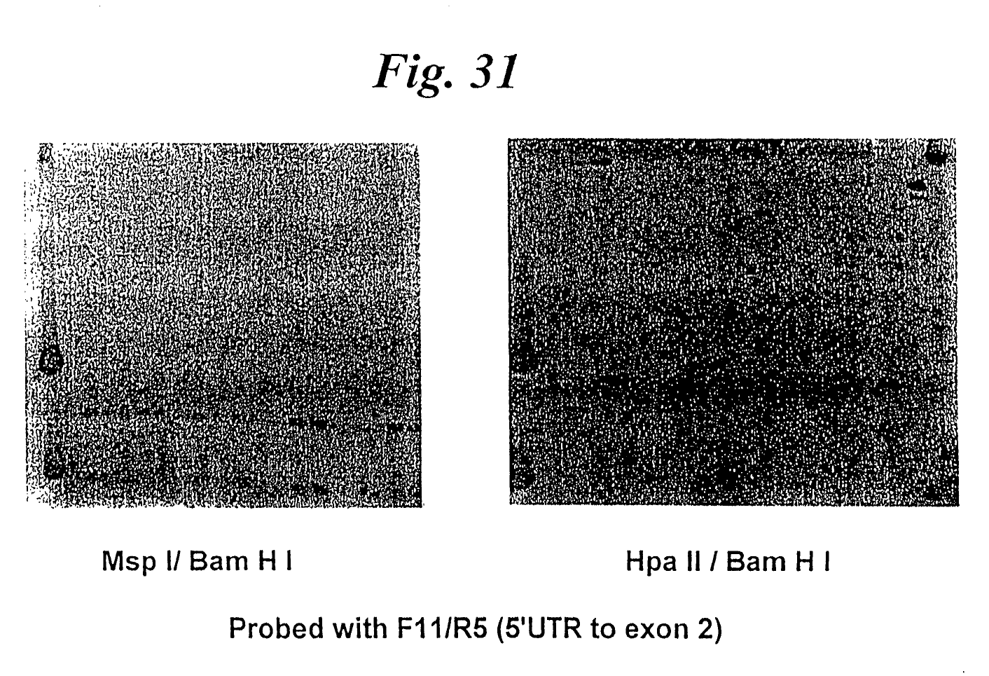

FIG. 31 shows Southern blots which indicate that the 5′ end of the Barx2 gene is methylated in ovarian cancer cell lines.

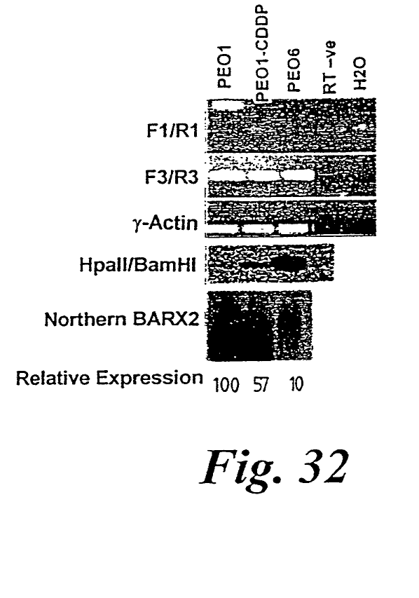

FIG. 32 shows that the extent of downregulation of Barx2 is proportional to HpaII methylation.

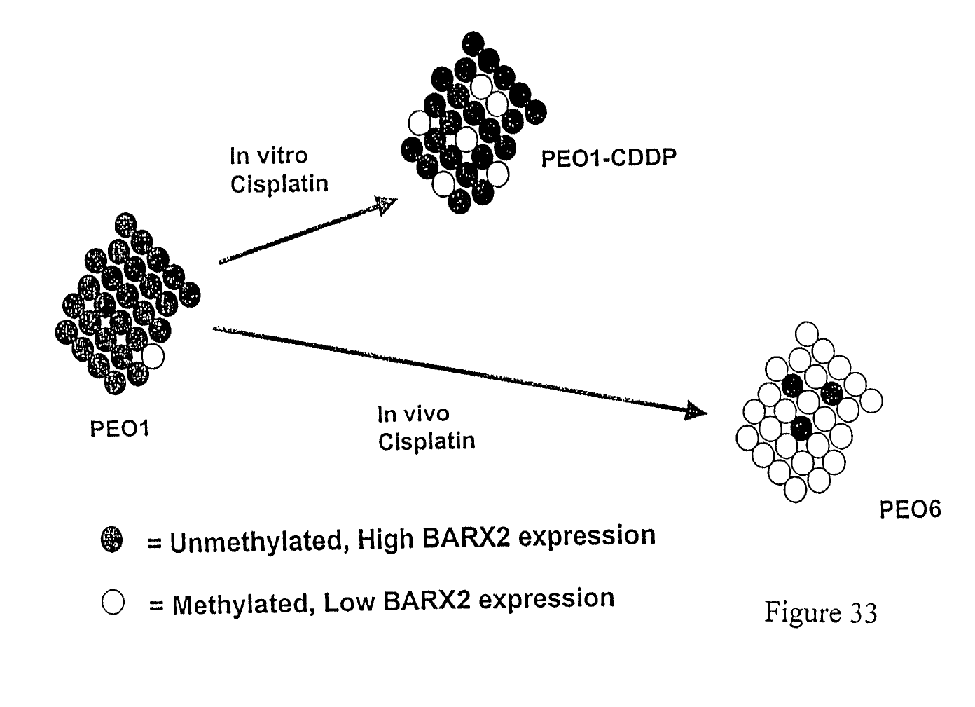

FIG. 33 proposes a clonal selection model to account for the accumulation of methylated, Barx2 down regulated, platinum resistant ovarian cancer clones.

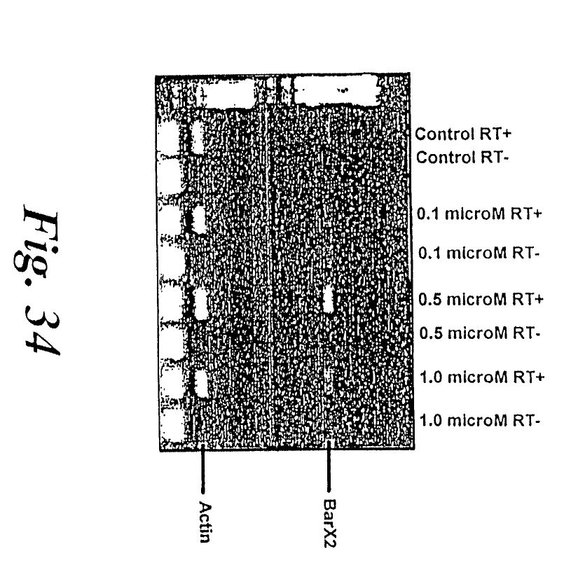

FIG. 34 shows that azacytidine demethylation of OAW42 re-expresses Barx2. OAW42 was exposed to 5-azacytidine for 96 hours at different concentrations. Clear induction of barx2 expression was observed at 0.5 μM azacytidine. This suggests that Barx2 is methylated, and that demethylation in OAW42 results in re-rexpression of barx”. Feint expression is seen for barx2 in OAW42 control, but strong induction of barx2 expression is seen at 0.5 μM azacytidine relative to actin signal.

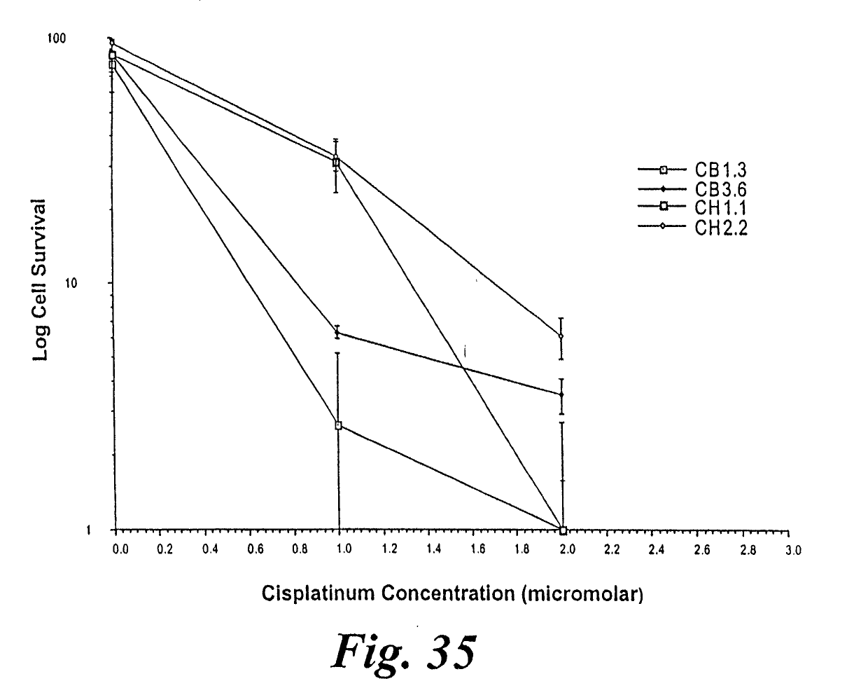

FIG. 35. The PEO1CCDP pBABE Hygromycin control transfectant cell lines CH1.1 and CH2.2, and the PEO1CDDP pBABE BARX2 transfectant cell lines CB1.3 and CB3.6, were assayed for survival at day 12 following 3 day exposure to 0 μM, 1.0 μM and 2.0 μM cisplatinum in a clonogenic assay. The increase in platinum sensitivity demonstrated following BARX2 transfection is statistically significant (p<0.0001) at 1.0 μM cisplatinum.

### EXAMPLE 1

### Functional Genetic Definition of a Chromosome 11 Tumour Suppressor Locus in Epithelial Ovarian Cancer

In this study, microcell mediated chromosome transfer of Chr 11 to a clonal subline of OVCAR3 ovarian cancer cells generated hybrids with whole or partial Chr 11 transfer. Transfer of whole Chr 11 exhibited morphological alteration, inhibition of Matrigel invasion, inhibition of attachment to a laminin coated surface and inhibition of Collagen IV mediated cell migration compared with controls. FACS analysis showed that Laminin Receptor levels were reduced. Chr 11q24 loss from this hybrid was associated with reversion of these phenotypes back to that of OVCAR3. The chromosomal region lost in this functional revertant overlapped the LOH region previously defined. Transfer of a fragmented Chr 11 with del (11) (q22-qter) was not associated with the above phenotypes although it was associated with inhibition of cell growth in vitro and in vivo, and inhibition of fibronectin-mediated cell migration, thus identifying a second functional locus.

The present studies attempt to define ovarian cancer chromosome 11 tumour-suppressors using a functional approach. Using the technique of microcell-mediated chromosome transfer (MMCT), we transferred a normal copy of human chr 11 from a murine somatic cell hybrid to OVCAR3, an ovarian cancer cell line with known disruption of chr 11. The fragments of transferred chr 11 retained within the resultant microcell hybrids were characterised using polymorphic microsatellites and fluorescence in situ hybridisation. The hybrids were then extensively analysed using functional assays: examining in vitro and in vivo growth; matrigel invasion, migration and attachment assays; annexin-V (apoptosis), cell cycle and integrin antibody analysis by FACS. “Suppressed” phenotypes were defined using these assays in the microcell hybrids with respect to the “malignant” state in OVCAR3 controls. Suppression of these malignant characteristics were correlated with retention of particular fragments of transferred exogenous chromosome 11 and demonstrate at least two functionally important loci, one within the 11q24 region mediating inhibition of invasiveness, and one outwith the 11q24 region, possibly within the 11p15 region, mediating growth suppression.

### Results

An overall summary of results from this study is presented in Table 1.

TABLE 1
Summary of phenotypes for controls and microcell hybrids in this study

| | Growth | Growth | | | Integrin | Migration | Migration | Migration |
|---|---|---|---|---|---|---|---|---|
| | in-vitro | in-vivo | Invasion | Adhesion | Analysis | (Collagen IV) | (Fibronectin) | (Laminin) |
| OH1 series | | | | | | | | |
| OH1 (Clonal hyg tagged line) | Control | Control | Control | Control | Control | Control | Control | Control |
| OHN (neo tagged OH1) | Control | Control | Control | Control | Control | Control | Control | Control |
| ONOH (OH1 MMCT using | Control | Control | Control | Control | NP | Control | Control | Control |
| ON1 donor) | | | | | | | | |
| 11OH1.1 (Whole 11 transfer) | suppressed | suppressed | NP | NP | NP | NP | NP | NP |
| 11OH1.3 (derived from | suppressed | suppressed | suppressed | Inhibited | ↑α3β1 | Inhibited | Inhibited | As Control |
| 11OH1.1 whole 11) | | | | (Laminin) | ↓LR-67 | | | |
| 11OH2.1 (del (11q22-qter)) | suppressed | suppressed | As | As | As | As Control | Inhibited | As Control |
| | | | Control | Control | Control | | | |
| 11OH2.4 | suppressed | suppressed | As | As | NP | NP | NP | NP |
| | | | Control | Control | | | | |
| 11OH1.2 (del (11q24), | NP | suppressed | As | As | As | As Control | As Control | As Control |
| revertant from 11OH1.1) | | | Control | Control | Control | | | |
| OHX series | | | | | | | | |
| OHXN (clonal neo tagged | Control | Control | NP | NP | Control | NP | NP | NP |
| OHX) | | | | | | | | |
| ONOHXN (MMCT from | Control | NP | NP | NP | NP | NP | NP | NP |
| ON1 to OHXN) | | | | | | | | |
| 11OHX1.1 (Whole 11 | suppressed | suppressed | NP | NP | α3β1 | NP | NP | NP |
| transfer) | | | | | unchanged | | | |
| | | | | | ↓LR-67 | | | |
| 11OHX2.1 | NP | NP | NP | NP | NP | NP | NP | NP |
| (del 11p13/11q13/11q22- | | | | | | | | |
| qter) | | | | | | | | |
| 11OHX2.2 (as 11OHX2.1) | suppressed | suppressed | NP | NP | As | NP | NP | NP |
| | | | | | Control | | | |
| 11OHX2.3 as 11OHX2.1 | As | As | NP | NP | NP | NP | NP | NP |
| with additional 11p15 loss | Control | Control | | | | | | |

### Microcell Fusion

For a full description of cell lines, including controls, see Methods.

Transfer of chromosome 11 was performed in order to assess its functional effects in OVCAR3. To minimise cell-line heterogeneity and artefacts of clonal selection, a hygromycin transfected clonal cell line, OH1, derived from OVCAR3, was used for microcell fusion. A second recipient OVCAR-3 derived cell line was generated by passage of OH1 as a murine subcutaneous xenograft, with subsequent in vitro rescue. This cell line was called OHX.

Neo-tagged, normal chr 11 was transferred by MMCT from MCH556.1.5 to OH1. Successful microcell mediated chromosomal transfer was confirmed by DNA in situ hybridisation using simultaneous Chr11p cosmid FISH and chr 11 paint in OHN, Hyg/Neo resistant clonal subline derived from OVCAR3. Both copy number abnormality and translocation/rearrangement involving both the short and long arms of chromosome 11 were seen. Microcell hybrid 11OH1.3 was seen to have a single additional copy of chromosome 11. Twelve microcell hybrid clones (MHCs) were obtained from 4 experiments. Twenty-three polymorphic microsatellites derived from the chr 11 radiation hybrid map (James et al, 1994) were used to map the extent of transferred chromosome in the hybrids. As can be seen, within each MMCT experiment, the derived MHCs seem largely similar by mapping, suggesting predominantly a single fusion event (FIG. 13).

Four MHCs contained an entire copy of the transferred chr 11 (eg 11OH1.3). Fortuitous partial transfers of chr 11 were observed in some MHCs with large deletions of distal 11q (11q22-qter); eg MHC 11OH2.1 and 11OH2.4. Additionally, a small deletion at 11q24 overlapping with the region defined by LOH (Gabra et al, 1996a) was noted in MHC 11 OH1.2, a revertant clone derived from MHC 11OH1.1.

### Growth-Analysis

### Morphology of Cells

Simple observation of the plating and growth characteristics of the control cells and microcell hybrids showed that the hybrids retaining the distal Chr 11 region (MHC11OH1.1 and MHC11OH1.3) grew as compact packed clusters and did riot spread to cover the surface of the plate rapidly, unlike the control lines and hybrids lacking distal Chr 11 region (FIG. 14a).

### In Vitro Cell-Growth

An important potential effect of a putative tumour suppressor is its capacity to inhibit growth, and a useful assay for this is an assessment of in vitro growth. The introduction of whole chr 11 into OH1 (MHC11 OH1.1) resulted in growth suppression in vitro. Hybrids containing Chr 11 lacking 11q22-qter (MHCs 11OH2.1-2.4) demonstrated equivalent growth suppression compared with the clone retaining whole Chr 11(OHN and ONOH, “neutral” chromosome transfer control).

Similarly, transfer of the donor Chr 11 to OHX demonstrated equal growth suppression for the hybrid retaining distal Chr 11 (MHC 11 OHX 1.1) and the hybrid which did not retain distal 11q (MHC 11OHX2.2). A hybrid with loss of the donated 11p15 region (MHC11OHX2.3) demonstrated accelerated growth suggesting that this could be the location of the growth suppressor.

This analysis suggested that the growth suppression effects were mediated by locus outwith 11q22-qter, possibly within 11p15.

### In vivo Subcutaneous Tumorigenicity

In vivo tumorigenicity assays were performed for two reasons. Firstly to see if the growth suppression effects in vitro correlated with suppression of tumorigenicity in vivo, and secondly to determine if the distal 11q region carried any differential suppression effects in vivo not observed by the growth assay in vitro.

For the microcell hybrids derived from OH1, transfer of chr 11 was associated with reduction in xenograft size relative to ONOH cell line but not with complete suppression of tumorigenicity and was similar between the hybrids, mirroring the in vitro growth findings. Histology of the xenografts by an experienced histopathologist suggested appearances of poorly differentiated adenocarcinoma and no histological differences were noted between the controls and the microcell hybrid xenografts. Introduction of chromosome 11 into the OHX cells also reduced their tumorigenicity. Interestingly, although whole chromosome 11 transfer (11OHX1.1) did retard growth of the OHX microcell hybrid xenografts, loss of chr 11 material from 11q22-qter (11q24) and 11p15 together (MHC 11OHX2.3) was associated with xenograft growth similar to controls potentially localising the ovarian cancer growth suppressor phenotype to 11p15 (ie, non-suppression of 11OHX2.3), a finding which correlates with in vitro assay.

In conclusion, the phenotypes of growth suppression and tumorigenicity suppression were not separable in this model system, and appear to map to the same locus, which possibly resides on distal 11p.

### Annexin-V and DNA Cell-Cycle FACS Analysis

In order to explain the growth inhibition observed above, further analysis was performed by several methods.

Cell-cycle analysis by FACS showed that chr 11 transfer was not associated with obvious alteration of the cell cycle compared to the OVCAR3 controls (data not shown).

Annexin-V FACS analysis (Vermes et al, 1995) was performed to assess if the growth delay was a function of apoptosis. The percentage of apoptotic cells noted for microcell hybrids (3-5 %) was the same as for control OHN (3%, p=n.s.) suggesting that apoptosis did not explain the observed phenomena(data not shown).

### Invasiveness Analysis

In view of the background observations leading to this work, ie that 11q24 LOH was frequently observed and was associated with poor prognosis in clinical ovarian cancer, further cell biological analysis was undertaken to demonstrate, if possible, differences between microcell hybrids retaining and losing distal 11q. One possible determinant of prognosis in ovarian cancer could be tumour cell invasiveness. Although it may correlate with tumorigenicity in some systems, this is not invariable, and the assays measure different endpoints.

### Matrigel Invasion Assay

The matrigel invasion assay measures the ability of cells to invade through a matrigel layer in vitro (Albini et al, 1987). MHC 11OH1.3 (containing whole transferred chr 11, and derived from MHC 11OH1.1) was shown to be significantly suppressed for invasiveness relative to the controls, MHC 11OH2.4 (large deletion of 11q) or MHC 11OH1.2 (small deletion of 11q) (FIG. 15a). In 4 separate experiments each in triplicate, MHC 11OH1.1(whole chromosome 11 transfer) was compared with MHC 11OH2.4 (small deletion of 11q24) and a highly significant abrogation of matrigel invasiveness was observed in the cell line retaining the 11q24 region (Mann-Whitney U-test p=0.0027, data not shown).

In conclusion, the matrigel invasiveness assay did allow the identification of significant differences in the capacity of the hybrids to invade, and this function mapped to the 11q24. Analysis of the hybrids shows that another region at 11p13 appeared to be co-deleted and its contribution to these phenotypes cannot be excluded.

### Cell Adhesion Assay

As mentioned above, the appearance of the MHC 11OH1.3 and MHC 11OH1.1 hybrids was different from the hybrids with deletions of 11q or the controls. Having identified differences in invasiveness, we considered the possibility that cellular adhesion could be a common factor underlying the differences noted by these two methods, and so the capacity of the controls and hybrids to adhere to tissue culture dishes coated with defined components of matrigel was assessed.

MHC 11OH1.1 and MHC 11OH1.3 (hybrids with whole chr 11 transfer) showed a rounded phenotype with a reduced propensity for spreading and migrating out to the edge of the tissue culture dish, particularly on laminin coated tissue culture plastic. This was in marked contrast to OHN (neo-tagged) and ONOH(neutral chromosome transfer) controls, MHC 11OH1.2 (with a small deletion of q24in the transferred chr 11), and MHC 11OH2.1 (with deletion of 11q22-qter ); all of which spread out quickly to cover the margins of the tissue culture dish (FIG. 14b).

The OH1-derived microcell hybrid clones were quantitatively analysed for their ability to attach to plastic surfaces coated with ECM proteins laminin, fibronectin and collagen IV. MHC 11OH1.3 (whole chr 11 transfer) was shown to be significantly inhibited in its ability to attach to laminin but not to fibronectin or to collagen. This was not the case for control cell lines or MHCs lacking a transferred 11q22-qter or the smaller 11q24 region (FIG. 15b).

### Integrin Analysis by Immuno-FACS

In view of the above observation that attachment to laminin but not fibronectin or collagen was altered in the hybrids retaining the 11q24 region, an analysis of expression of putative laminin receptors both integrin and non-integrin was performed. Antibodies to a range of integrins (Table 2) that had been observed to function as laminin receptors in other contexts were selected for FACS analysis to determine if the observed functional alterations correlated with integrin expression profiles of the controls and hybrids.

TABLE 2
Expression of Laminin Receptor by FACS analysis in
microcell hybrids compared with controls

| | LR-67 expression | P-value compared to |
|---|---|---|
| Cell Line | relative to control | control control |
| MHC 11OH1.3 | 50% of control | <0.0001 |
| MHC 11OH1.2 | As Control | Not significant |
| MHC 11OH2.1 | As Control | Not significant |
| MHC 11OHX1.1 | 75% of control | 0.047 |

Comparing the OHN control cell line with MHC 11OH1.3 hybrid (whole Chr 11 transfer) demonstrated that expression of the non-integrin laminin receptor LR-67 was significantly reduced in the 11OH1.3 hybrid (FIG. 16). There was no difference in laminin receptor expression between MHC 11OH2.1 (del 11q22-qter) and OHN. Comparison of MHC 11OH1.3 with MHC 11OH1.2 (revertant from 11OH1.1 lacking 11q24) showed that MHC 11OH1.2 had significant relative over-expression of LR-67, providing supportive evidence that a locus within 11q24 may be associated with downregulation of LR-67.

Further analysis of the OHX hybrid series in 4 independent experiments showed that MHC 11OHX1.1 (whole Chr 11 transfer to OHX) had significantly reduced expression of LR-67 compared with OHXN control, although the magnitude of reduction of expression was less than that seen in the OH1 series, and this may be attributable to in vivo passaging. This data is summarised in Table 2.

α3β1 expression was significantly elevated in 11OH1.1 relative to controls, but no such difference was found in the OHX hybrid series, suggesting that this may be a spurious observation.

In conclusion, the immuno-FACS experiments demonstrated reduced expression of LR-67 in hybrids retaining distal 11q and provided supportive evidence for the observation of reduced attachment to laminin associated with this region.

### Cell Migration Analysis

Having identified differences in the cytological appearance of the hybrids, we also noted that the 11OH1.1 and 11OH1.3 did not spread as efficiently as controls or hybrids with disruption of 11q.

The purpose of the cell migration assay was to quantify the haptotactic migratory response to purified ECM components in order to assess the effect of chr 11 introduction on this process. The OH1-derived MHCs were analysed for their ability to migrate in response to laminin, fibronectin or collagen IV. MHC 11OH1.3 was shown to be significantly inhibited in its ability to migrate in response to collagen compared with controls and other hybrids lacking the 11q24 region. Both 11OH1.3 and 11OH2.1 (with a large deletion of 11q) were inhibited in their capacity to migrate towards a fibronectin haptotactic signal equally, suggesting a second locus affecting cell migration located outwith the 11q22-qter region.

### Homozygous Deletion Mapping

The MMCT hybrid 11OH1.2 was shown by microsatellite analysis to have lost a discrete 4.5 Mb region at 11q24 in association with reversion of the invasiveness/attachment/migration inhibited phenotype observed for hybrids 11OH1.1 and 11OH1.3.

### Expression of Candidate Chr 11 Genes

We considered the possibility that other chromosome 11 candidate tumour suppressor genes could contribute to the neoplastic state in OVCAR3 and that the expression of these genes could produce suppression in the microcell hybrids.

Expression levels of TSG101at 11p15 (Li & Cohen, 1996; Matsuoka et al, 1995), WT-1 at 11 p13 (Call et al, 1990; Dowdy et al, 1991) and KAI-1 at 11p11 (Dong et al, 1995) were analysed by RT-PCR. Expression of these three genes was detected in the OHN cell line and all the microcell hybrids and did not correlate with the observed phenotypes (data not shown). CD44 (11p13) and N-CAM (11q22) protein expression was analysed using antibodies in FACS analysis (Table 3). Again, no significant differences were noted between the hybrids that correlated with the observed phenotypes.

### Discussion

Subcutaneous tumorigenicity assays in nude or SCID mice have by default become the standard assay to test for a functional tumour suppressor gene (Harris et al, 1969; Saxon et al, 1986) This assay, however, often identifies only powerful suppressors, and usually this is entirely out of context for the malignant disease process in question. In pursuit of novel tumour-suppressor genes, we have been directed by a clinical observation in which 11q24 LOH was associated with adverse 5 year survival for patients with ovarian cancer. We have investigated the effect of the re-introduction of chromosome 11 into a clonally derived subline of OVCAR3 (a human ovarian cancer cell line with observed rearrangement of the telomeric portions of 11q and 11p) on the phenotype of this cell line.

The use of a clonal subline and the derivation of multiple clonal controls derived by both transfection and microcell fusion reduce the chance that these observations are artefacts of clonal selection.

The functional analyses presented here suggest possible mechanisms of action for the putative tumour suppressor gene(s) which parallel the pathophysiological mechanisms associated with clinical ovarian cancer progression.

Others (Rimessi et al, 1994) have also previously identified suppression of growth of ovarian cancer cells associated with chromosome 11 transfer without alteration of tumorigenicity. However, in view of the initial clinical observation of poor prognosis for the ovarian cancer cohort exhibiting LOH at 11q24, we have performed a more detailed functional analysis.

In this study, microcell fusion has identified a region on chr 11 which suppressed growth in vitro and in vivo. However, microsatellite analysis showed that this region conferring growth inhibition was located outside

both the 11q22-qter and 11p13 region. Interestingly, microcell hybrid 11OHX2.3 showed accelerated growth and tumorigenicity in association with loss of the 11p15 region, raising the possibility that this could be the location of the growth/tumorigenicity suppressor. The growth inhibition phenotype was shown not to be due to an obvious cell-cycle or apoptosis effect.

Transfer of whole chr 11, but not chr 11 (del q22-qter) appears to confer a coordinated phenotype involving suppression of the capacity in vitro to invade into matrigel, reduction in the ability to attach to laminin, and reduction in the ability to migrate towards a collagen IV-mediated haptotactic signal.

Hybrid 11OH2.1 with loss of 11q22-qter exhibited inhibition of migration towards a fibronectin haptotactic signal, and this inhibition was of the same magnitude as that seen in 11OH1.1 and 11OH1.3 (retaining distal 11(q). This suggests a second inhibition of migration locus outwith distal 11q. Our analysis does not allow us to separate this migration-inhibition locus from the growth/tumorigenicity suppression loci discussed above, and further analysis is required in order to unify these phenotypes.

A microcell hybrid clone which had lost the invasion/migration/attachment suppressed phenotype despite apparent whole chr 11 transfer was found to have a small region of loss at 11q24 which directly overlapped the region defined both by our clinical LOH analysis (Gabra et al, 1996a) and that of others (Davis et al, 1996). Disruption of this putative locus in vivo could clearly be an important factor for intraperitoneal dissemination and formation of peritoneal metastases in ovarian cancer, and could be responsible for the previously observed phenotype of adverse survival.

Increased expression of the laminin receptor has been correlated with adverse prognosis (Basolo et al, 1996; Pellegrini et al, 1995). We have observed reduced expression of laminin receptor (located on Chr 3p21.3 (Jackers et al, 1996)) in association with microcell-mediated introduction of the distal 11q region and its concomitant suppressed phenotype, which is consistent with the above clinical observations and raises the possibility that the chr 11q suppressor may directly regulate laminin receptor-expression.

We have shown using RT-PCR analysis that none of 4 candidate TSGs located on chromosome 11 (WT1, TSG101, KAI-1, NCAM and CD44) showed alterations of expression associated with chr 11 re-introduction, suggesting that they are not involved in the malignant phenotype observed in OVCAR3.

Although the identification of a small homozygous deletion in this region would have assisted a positional approach to identify the putative 11q tumour suppressor, no such deletion was identified despite an extensive search using 15 markers from the region and 88 independent human tumour cell lines.

As is described in more detail in the following Examples, the tumour suppressor gene has been identified as Barx2.

### Methods

### Microcell Mediated Chromosome Transfer

MCH556.1.5 (obtained from Eric Stanbridge via A. G. Jochemsen (Leiden)) is a mouse-human somatic cell hybrid containing human chr 11, with neo insertion at 11q14-q22, maintained in DMEM/ 10% FCS/penicillin/streptomycin/G418 600 μg/ml.

Briefly, 60-70% confluent MCH556.1.5 cells were exposed to colcemid (Demecolcine, Sigma) 75 ng/ml in DMEM for 48 hr to micronucleate the cells, reuspended in Percoll (Pharmacia) /68 mM Na Cl/ pH 7.2 (22.5 mM HEPES):10% FCS/DMEM, 1:1 with 20μg/ml cytochalasin B (Aldrich).

Cells were centrifuged at 19,000 rpm for 70 minutes at 34° C., diluted in serum-free media and filtered through a 3 μm Nucleopore polycarbonate filter in a dual membrane stirred cell holder. The filtered microcells were pelleted at 1300 rpm for 15 minutes at room temperature and resuspended in 2.5 ml Hanks balanced salt solution/25 mM HEPES pH 7.2 and 100 μg/ml Phytohaemagglutinin-P. The microcells were layered on the recipient cells and left to attach for 20 minutes at room temperature, aspirated and 2ml pre-warmed polyethylene glycol in 75 mM HEPES (sterile, fusion tested, Boehringer) was added for 60 seconds to fuse the lipid membranes of the microcells with recipient cells. The recipient monolayer was washed 3 times with serum free DMEM and left in non-selective media for three days. The cells were re-seeded into a T175 flask for 24 hours in non-selective media and then dual selection with G418 at 325 μg/ml and hygromycin-B at 75 μg/ml was applied until no cells remained in the control flasks. MCH clones were picked at 3-6 weeks.

DNA was extracted for microsatellite analysis at the earliest possible passage, usually passage 3-4.

### Cell Lines

Cell lines were maintained in DMEM/10%FCS/Penicillin/Streptomycin with selective media (HygromycinB and G418 as appropriate).

OH1 is a clonal Hyg^{r} OVCAR3 subline derived by transfection of tgCMV/HgTK (Lupton et al, 1991) into the ovarian adenocarcinoma cell line OVCAR3 (Hamilton et al, 1983). OVCAR3 and OH1 have a hypertriploid karyotype with re-arrangement of chr 11.

OHX was a cell line derived by recovering OH1 cells grown once in a nude mouse as a subcutaneous xenograft tumour.

Controls were derived by transfecting pMC1neoPolyA (Thomas & Capecchi, 1987) into clonal OH1 and OHX cell lines thereby deriving Hyg/Neo-resistant clonal cell lines OHN and OHXN respectively.

OVCAR3 was transfected with pMC1neoPolyA and a clonal line ON1 was derived. ON1 was then used to transfer a neo-tagged chromosome to OH1 (ONOH) and OHX (ONOHX) by MMCT. Empirially these control cell lines behaved identically across the range of assays employed in this study (data not shown).

### Simultaneous Chromosome FISH/Paint

Metaphase spreads of cell lines and MHCs were prepared using essentially standard methods (Watson et al, 1995).

15 μl Chromosome paint (STAR*FISH, Cambio) ( pre-warmed, 42° C.) was aliquoted for each slide and denatured by incubating at 65° C. for 10 minutes. Pre-annealing was performed by incubating at 37° C. for 15-60 minutes.

Metaphase slides were denatured in 70% formamide/ 2×SSC at 70° C. for 2 minutes, quenched in ice cold 70% ethanol, dehydrated through 90% and 100% ethanol at room temperature for 3 minutes and air dried.

The paint was placed on a pre-warmed coverslip, sealed onto pre-warmed slides and incubated overnight at 42° C.

The slides were soaked in 2×SSC at 42° C. until the coverslip floated off, washed twice in 0.5×SSC: 50% formamide (1;1 mix of formamide+1×SSC) at 42° C. for 5 minutes, then washed twice in 2×SSC at 42° C. for 5 minutes. Finally the slides were mounted in 40 μl Vectashield with DAPI and PI (3.75 μl 100 &g/ml DAPI+3.75 μl 20 ug/ml PI in 100 μl Vectashield), visualised using a fluorescence microscope and analysed using a Mac-based software analysis system.

### Microsatellite Mapping of MMCT Hybrids

DNA was extracted using the QIAamp DNA extraction kit (Qiagen) according to manufacturer's instructions.

Oligonucleotides primers were selected from a high resolution radiation hybrid map (James et al, 1994). One primer of each pair was fluorescently labelled for analysis on the automated laser fluorescence system (ALF system, Pharmacia).

A standard PCR programme was used on an Omni block PCR machine (Hybaid): (94° C. for 3 min.)×1; (94° C. for 30 sec., 55° C. for 30 sec., 72° C. for 1 min)×35. 0.5-2 μl PCR products were separated on 6% acrylamide/7M urea /1×TBE gel at 50 watts for 180 minutes at 40° C.

### Cell-Growth Experiments

Log phase cultures were harvested and 10⁴ cells were seeded in 24 well trays. Cells were harvested every 2 or 3 days depending on the cell line for counting using a standard coulter counter protocol.

### Subcutaneous Tumorigenicity Assay

10⁷ cells were harvested, washed, pelleted and resuspended in 250 μl 10% FCS (protease stripped) media, mixed with 250 μl matrigel (Beckton Dickinson), sub-cutaneously injected into SCID mice. Tumour volumes were calculated weekly based on bi-dimensional tumour diameter measurements.

### DNA Cell-Cycle and Annexin-V FACS Analysis

A single cell suspension was prepared. Cells were fixed and stained with propidium iodide and analysed on a Becton Dickinson FACScan. Relative DNA content and distribution of cells with respect to the cell cycle was assessed.

Flow-cytometric detection of phosphatidylserine expression in cell lines and MHCs was performed according to the manufacturer's method using the Fluorescein labelled Annexin-V Apoptosis Detection Kit (R&D systems) (Vermes et al, 1995). The measure of early apoptosis derives from the assessment of the proportion of Annexin-V positives Propidium Iodide negative cells.

### Matrigel Invasion Assay

Pre-aliquoted Matrigel (Beckton Dickinson) was thawed on ice and diluted 1:5 in ice cold pre-treated culture medium.

140 μl of cold diluted matrigel was aliquoted into Transwell cell culture chambers (Costar) with inserts containing 12 μm pore polycarbonate membrane (Nucleopore). The matrigel was evenly distributed by tilting and allowed to gel by incubating at 37° C. for 30 minutes.

10⁵ cells (washed in protease inhibitor stripped 10% FCS DMEM) were dispensed into the upper compartment and incubated in a humidified incubator for 48 hours.

The number of cells on the upper and under-surface of the porous membrane were assessed using the MTT assay (Imamura et al, 1994).

Statistical analysis was performed using the In-Stat program (Graphpad 5 software), utilising the Mann-Whitney U test and Kruskal-Wallis nonparametric ANOVA test.

### Quantitative Adhesion Assay

Tissue culture plastic was pre-blocked using albumin. Extra-cellular matrix proteins (fibronectin, laminin, collagen IV) were diluted in PBS to between 2 and 50 μg/ml. 50 μl was added to wells and the 96-well tray was incubated overnight at 4° C. The plate was then washed twice with PBS to remove unbound protein. 200 μl 0.1% w/v BSA in PBS/0.1% azide was added to each well, and incubated at 37° C. for 2 hours.

70% confluent cells were harvested as a single cell suspension and resuspended in 10% FCS/DMEM. 90 μCi radioactive chromate (Sodium Chromate[51Cr], Amersham) in 100 μl 10X Hanks buffered-salt solution was added to the cells, incubated at 37° C. for 60 minutes. The cells were washed 3 times and resuspended at 2×10⁵ viable cells (trypan blue estimation) per ml in serum free media.

Plates were washed twice in PBS, placed on ice and the labelled cells were immediately added (10⁴ cells in 50 μl per well) and incubated for 2 hours at 37° C. Plates were gently washed to eject unbound cells and slowly immersed in PBS(1 mM Ca²⁺/0.5 mM Mg²⁺) The amount of radioactivity remaining was counted allowing a calculation of percentage adhesion.

The In-Stat program (Graphpad software) was used for statistical analysis, utilising a one-way analysis of variance followed by the Tukey-Kramer multiple comparisons test for the simultaneous adhesion assay using 4 different cell lines and three different ECM haptotactic signals.

### Integrin Analysis

Cells were trypsinised and resuspended at 10⁶/ml in complete medium and incubated at 37° C. to allow receptors to recover prior to analysis. Aliquots of 0.5×10⁶ cells were washed in PBS and again in PBS/5% FCS (FCS/PBS) before incubation at 4° C. for 60 minutes with the appropriate integrin antibody at the appropriate concentration (previously estimated empirically, Table 3). After a further wash in FCS/PBS cells were incubated at 4° C. for 60 minutes with 1:40 dilution of rabbit anti-mouse phycoerithrin conjugate (Dako) in FCS/PBS then washed again in FCS/PBS and resuspended in 1 ml PBS for analysis on a Becton Dickinson FACScan. Median values of red fluorescence were recorded and expressed as a ratio for each receptor over background fluorescence (measured by omitting the primary antibody).

Several independent samples were undertaken in each experiment using the OHN series. Statistical analysis was performed using the Mann-Whitney U test.

In the OHX series triplicate values were derived from single samples in each experiment, and four separate experiments were performed. In order to compare between experiments, controls were normalised to 100% and hybrids were given % expression values relative to this. Since no standard deviation could be derived from the controls between experiments (all 100%) a one sample t-test had to be performed.

TABLE 3
Antibodies used for Flow-cytometric analysis

| Integrin | Supplier | Clone | Dilution |
|---|---|---|---|
| α2β1 | Dako | P1E6 | 1:40 |
| α3β1 | Dako | P1B5 | 1:40 |
| α6 | Chemicon | NK1-GoH3 | 1:10 |
| β4 | Chemicon | 3E1 | 1:200 |
| αVβ3 | Chemicon | LM609 | 1:50 |
| β1 | Chemicon | JB1 | 1:50 |
| LCAM | Europath | 5H9 | 1:10 |
| NCAM | Europath | RNL-1 | 1:10 |
| Laminin Receptor | Genzyme | MLuC5 | 1:50 |
| CD44 | Dako | DF1485 | 1:40 |

### Transwell Migration Assay

ECM proteins were immobilised on the lower surface of the polycarbonate membrane by incubating the under-surface of 8.0 μm pore-size transwell cell culture inserts (Costar) in a 24 well plate with 250 μl of 10 μg/ml solution of the ECM component at 37° C. for 1 hour. The transwells were blocked by transferring the transwell to a 24 well plate with BSA 0.1% for 1 hour at room temperature. The transwell under-surface was washed twice by replacing BSA with PBS. 400 μl serum-free DMEM were applied to the lower compartment. 5×10⁴ cells in a final volume of 100 μl serum-free DMEM were applied to the top compartment of the transwell. The cells were allowed to migrate across the membrane at 37° C. for 72 hours. Non-adherent cells were removed by rinsing the upper chamber twice with PBS. MTT was added to the top and bottom compartments of the transwell according to a standard protocol (Imamura et al, 1994) and percentage migration was quantified and compared with control plates (transwells coated with 0.1% BSA only on their under-surface). Statistical analysis was performed using the In-Stat program (Graphpad software), using the Mann-Whitney U test, Kruskal-Wallis nonparametric ANOVA test with Dunn's multiple comparison test to. indicate the significance of the differences between the sample.

### Homozygous Deletion Mapping

Fluorescein labelled primers were used for PCR of 88 cancer cell lines using markers across the minimal 11q24 region defined by hybrid MHC 11OH1.2. The markers derived from various world wide web genome database resources. 1-2 μl of PCR product were loaded onto polyacrylamide gels for analysis using the ALF system (see microsatellite analysis above) using standard PCR protocols. The markers used (from centromeric to telomeric) were: WI-7244, D11S4131, D11S4126, NIB1699, WI-9552, D11S912, WI-9884, D11S669, D11S1884, D11S910, D11S1894, D11S874, D11S1320, D11S969.

### Reverse Transcriptase-PCR Analysis

Cell lines and MHCs were grown to 70% confluence and then harvested. Total RNA was extracted from 4×10⁵ cells using the Tri-reagent kit (Molecular Research Center, inc., Cincinnati). The method was according to manufacturer's instructions. Contaminating DNA was removed using RNase-free DNase-1. Oligo-dT primed cDNA was prepared using MMLV RT (Promega) and resuspended in 100 μl TE(10 mM Tris pH8.0, 1 mM EDTA). Aliquots of 10 μl were amplified using Taq polymerase (Perkin-Elmer-Cetus) and primers specific for KAI-1( ), TSG101( ), and WT-1 ( ), all using the following program: 35 cycles with denaturing at 94° C. for 1 min, annealing at 55° C. for 1 min and extension at 72° C. for 1 min. Controls with water only and without RT ensured that all bands observed were specific. Products were separated on 2% agarose and stained with ethidium bromide.

### References for Example 1

Albini, A., Iwamoto, Y., Kleinman, H. K., Martin, G. R., Aaronson, S. A., Kozlowski, J. M. & McEwan, R. N. (1987) Cancer Res, 47, 3239-45.

Basolo, F., Pollina, L., Pacini, F., Fontanini, G., Menard, S., Castronovo, V. & Bevilacqua, G. (1996) Clin Canc Res. Clinical Cancer Research, 2, 1777-1780.

Call, K. M., Glaser, T., Ito, C. Y., Buckler, A. J., Pelletier, J., Haber, D. A., Rose, E. A., Kral, A., Yeger, H., Lewis, W. H. & et, a. (1990) Cell, 60, 509-20.

Davis, M., Hitchcock, A., Foulkes, W. D. & Campbell, I. G. (1996) Cancer Res, 56, 741-744.

Dong, J. T., Lamb, P. W., Rinkerschaeffer, C. W., Vukanovic, J., Ichikawa, T., Isaacs, J. T. & Barrett, J. C. (1995) Science, 268, 884-886.

Dowdy, S. F., Lai, K. M., Weissman, B. E., Matsui, Y., Hogan, B. L. & Stanbridge, E. J. (1991) Nucleic Acids Res, 19, 5763-9.

Gabra, H., Watson, J. E. V., Taylor, K. J., MacKay, J., Leonard, R. C. F., Steel, C. M., Porteous, D. J. & Smyth, J. F. (1996a) Cancer Res, 56, 950-954.

Hamilton, T. C., Young, R. C., McKoy, W. M., Grotzinger, K. R., Green, J. A., Chu, E. W., Whang-Peng, J., Rogan, A. M., Green, W. R. & Ozols, R. F. (1983) Cancer Res, 43, 5379-89.

Harris, H., Miller, O. J., Klein, G., Worst, P. & Tachibana, T. (1969) Nature, 223, 363-8.

Imamura, H., Takao, S. & Aikou, T. (1994) Cancer Res, 54, 3620-4.

Jackers, P., Minoletti, F., Belotti, D., Clausse, N., Sozzi, G., Sobel, M. E. & Castronovo, V. (1996) Oncogene, 13, 495-503.

James, M. R., Richard III, C. W., Schott, J.-J., Youstry, C., Clark, K., Bell, J., Terwilliger, J. D., Hazan, J., Dubay, C., Vignal, A., Agrapart, M., Imai, T., Nakamura, Y., Polymeropoulos, M., Weissenbach, J., Cox, D. R. & Lathrop, G. M. (1994) Nature Genet., 8, 70-76.

Li, L. & Cohen, S. N. (1996) Cell. Cell., 85, 319-329.

Lupton, S. D., Brunton, L. L.,, Kalberg, V. A. & Overell, K. W. (1991) Molec Cell Biol, 11, 3374-3378.

Matsuoka, S., Edwards, M. C., Bai, C., Parker, S., Zhang, P., Baldini, A., Harper, J. W. & Elledge, S. J. (1995) Genes Dev, 9, 650-62.

Pellegrini, R., Martignone, S., Tagliabue, E., Belotti, D., Bufalino, R., Cascinelli, N., Menard, S. & Colnaghi, M. I. (1995) Breast Cancer Res Treat, 35, 195-9.

Saxon, P. J., Srivatsan, E. S. & Stanbridge, E. J. (1986) Embo J, 5, 3461-6.

Thomas, K. R. & Capecchi, M. R. (1987) Cell, 51, 503-512.

Vermes, I., Haanen, C., Steffens-Nakken, H. & Reutelingsperger, C. (1995) J Immunol Methods, 184, 39-51.

Watson, J. E. V., Slorach, E. M., Maule, J., Lawson, D., Porteous, D. J. & Brookes, A. J. (1995) Genome Res, 5, 444-452.

### EXAMPLE 2

### Expression of the Barx2 Gene shows Suppression of Growth in Certain Ovarian Cancer cell Lines, and Inhibition of Cellular Invasiveness into Matrigel

FIG. 6 shows two PCR primer reactions. The upper band is genomic PCR of the Barx2 cDNA (ie no introns), and therefore detects non-endogenous Barx2 copies. The lower band represents hygromycin PCR, and this reflects the presence of vector sequence conferring hygromycin resistance. The clones were selected for on hygromycin containing media (75 μg per ml final concentration hygromycin in DMEM with 10% serum, and antibiotics penicillin/streptomycin). At the top of FIG. 6 are listed the different clones derived from this experiment. “B” refers to OVCAR3 clones transfected with Barx2 plasmid (pBabe BARX2). “CH and EH” refer to OVCAR3 clones transfected by the control hygromycin plasmid pBabeHygro. As can be seen, all clones growing in hygromycin were positive for hygromycin transfected sequence. None of the control plasmid transfectants had evidence of Barx-2 cDNA. BX3.2 and 3.4 had very low copy number of the Barx-2 cDNA, suggesting that the clone had disrupted the Barx2 sequence in almost all the cells. The remaining clones had clear evidence of non-endogenous Barx2 cDNA by PCR suggesting successful transfection of the BARX2 plasmid into these clones.

Several of these clones were then tested in growth experiments (FIGS. 15 and 16). 10,000 cells were plated in 24 well plates and cells were counted every three days using a coulter counter. The Hyg transfected control lines grew rapidly and BX3.4, which did not have evidence of Barx2 sequence in FIG. 6, also grew with the same characteristics. BX3.4, with evidence of lower copy number transfection grew more slowly, but not significantly so. BX3.6 and 4.2 which had clear evidence of Barx2 plasmid transfection grew significantly more slowly than the other lines suggesting that Barx2 inhibits cell growth in OVCAR3. This oservation has been extended to PEO1CDDP cell line.

FIG. 8 shows data from “invasion Bx”. This assay is based on the principle that the basement membrane plays an important part as a barrier against tumour cell invasion.

Pre-aliquoted Matrigel (Beckton Dickinson) was thawed on ice and diluted 1:5 in ice cold pre-treated culture medium.

140 μl of cold diluted matrigel was aliquoted into Transwell cell culture chambers (Costar) with inserts containing 12 μm pore polycarbonate membrane (Nucleopore). The matrigel was evenly distributed by tilting and allowed to gel by incubating at 37° C. for 30 minutes.

10⁵ cells (washed in protease inhibitor stripped 10% FCS DMEM) were dispensed into the upper compartment and incubated in a humidified incubator for 48 hours.

The number of cells on the upper and under-surface of the porous membrane were assessed using the MTT assay.

Statistical analysis was performed using the In-Stat program (Graphpad software), utilising the Mann-Whitney U test and Kruskal-Wallis nonparametric ANOVA test.

Using this method, the Barx2 transfected clones were shown to be severely suppressed for matrigel invasiveness, and this result is consistent with the finding that the microcell hybrid MCH 11OH1.3 which retained the region that includes Barx2 was also suppressed for invasiveness (see microcell hybrid paper rejected by oncogene). The survival of patients with ovarian cancer as previously noted was worse when LOH of the 11q24 region was subject to LOH. Disruption of Barx2 could therefore accelerate the capacity of ovarian cancer cells to invade, and these invasion experiments suggest that this hypothesis is entirely plausible.

### EXAMPLE 3

### Northern Blot Analysis Shows that Several Ovarian Cancer Cell Lines do not Express Barx2

Northern Blot of ovarian cancer cell lines. 10 μg RNA for each sample. Probed with 32P dCTP random primed BARX2 cDNA. Shows abundant expression in PEO1 and downregulated expression in PEO4 and PEO6. An explanation of these cell lines is given above. PEO6 was derived a few months before this patient died, at a later time point to when PEO4 was obtained.

OVCAR3 expresses BARX2 transcript abundantly but OAW42 and A2780 do not apparently express BARX2 at all on Northern.

FIG. 3 shows a northern blot which indicates differential expression of Barx2 in ovarian cancer cell lines. See also FIG. 5.

### EXAMPLE 4

### A 50 bp Insertion Containing a Stop Codon is Found in a Barx2 Transcript

### Human Barx2 RT-PCR (Reverse Transcriptase-Polymerase Chain Reaction) and Mutation Detection by Single Strand Conformation Polymorphism (SSCPE).

Total RNA was isolated from the cell lines detailed in the FIG. 8 using TRI Reagent (Sigma) as per the manufacturer's protocol. First strand cDNA was then reverse transcribed using oligo dT as a primer and total RNA from the respective cell lines as template using the First Strand cDNA Synthesis Kit (Roche) as per standard protocols.

PCR reactions were performed on 1st strand cDNAs using combinations of primers that would permit generation of overlapping PCR products derived from the human Barx2 transcript. In addition, the full length coding region of the transcript was also analysed by PCR using the HBARX2F1 in combination with the HBARX2F3. Appropriate RT negative and water controls were included as appropriate in experiments.

| | | Nucleo- | |
|---|---|---|---|
| | | tides | |
| | | (AF031924 | |
| | | Number- | Ex- |
| Primer Name | Primer sequence | ing) | on |
| HBARX2 F1: | 5′-ATGATCGACGAGATCCTCTC-3′ | 170-189 | 1 |
| HBARX2 F2: | 5′-CACCGAGGCGGTCTCTGCTG-3′ | 400-419 | 2 |
| HBARX2 F3: | 5′-TGGTATCAGAATCGCAGGAT-3′ | 632-651 | 3 |
| HBARX2 R1: | 5′-GTGTTCCGTCTCTGACTCGC-3′ | 469-450 | 2 |
| HBARX2 R2: | 5′-GCTTCCTGTCCACCTTTAAG-3′ | 690-671 | 4 |
| HBARX2 R3: | 5′-GCTTAATGGTGGGGGTTCCG-3′ | 931-912 | 4 |

Exon 1 Primer: F11 which can substitute for F5 in
Exon 1 PCR

| | | | |
|---|---|---|---|
| HBARX2F11: | 5′-TCACCATGCACTGCCACG-3′ | 92-109 | 1 |

Human BARX2 Primer combinations for RT-PCR and SSCPE are given below:

| Primers | Product size |
|---|---|
| HBARX2F1/HBARX2R1 | 300 bp |
| HBARX2F2/HBARX2R2 | 291 bp |
| HBARX2F3/HBARX2R3 | 300 bp |
| HBARX2F1/HBARX2R3 | 762 bp |
| HBARX2F11/HBARX2R1 | 378 bp |

PCR products were then separated by size through agarose containing ethidium bromide and visualised under ultra-violet light following standard gel electrophoresis protocols.

FIG. 8 shows the result of RT-PCR of cell line RNAs using the primer combination HBARX2F11I/HBARX2R1 for the 5′-end of the BARX2 transcript. The product from cell lines PEO6, PEO16 and OHN are wild type in size. The F1/.R1 PCR product from OVCAR3 is larger than predicted, and the Microcell Hybrid (MCH) contains both the wild type and larger product. This larger than predicted product (a ˜50 bp insertion) was confirmed by separation on a denaturing polyacrylamide gel as for SSCPE. The nature of the larger product was characterised by sequencing.

The HBARX2F1/HBARX2R1 RT-PCR product from OVCAR3 was subcloned into the cloning vector pGEM-T and sequenced using the T7 sequencing primer. The sequence generated initiates from with the pGEM-T vector sequence, covers from the R1 primer to the F1 primer and then extends back into the cloning vector. With reference to FIG. 12(a) the pGEM-T cloning vector sequence is in plain text, the BARX2 sequence from HBARX2R1 to HBARX2F1 is underlined, and the 48 bp of inserted sequence with respect to the database sequence AF031924 is both underlined and italicised.

HBARX2 F1/R1 PCR Product from OVCAR3 subcloned into pGEM-T and sequenced with T7 primer.

Product is ⁻50 bp (48 bp) larger than predicted from AF031924 HBARX2 mRNA sequence.

The F1/R1 nucleotide sequence obtained from OVCAR3 was translated using using the ExPasy translation programme. The sequence encoded by exon 1 as detailed in the AF031924 sequence is underlined. The sequence then predicts a ‘stop’ codon downstream resulting either in the production of a shortened form of the BARX2 protein that lacks the homeodomain that is therefore non-functional or is altered in function. Alternatively, no protein product may be produced in the cell.

ExPasy Translation of HBARX2F1/R1 RT-PCR product from OVCAR3 is shown in FIG. 12(b).

A CLUSTAL W (1.74) multiple sequence alignment of OVCAR3 HBARX2 F1/R1 translation (BARX2) with protein database sequence of human BARX2 (043518) was performed. The protein sequence beyond that encoded by exon 1 breaks down in keeping with the insertion of additional nucleotides in the OVCAR3 Human BARX2 F1/R1 RT-PCR product (see FIG. 12(c)). Hence, the 48 bp insertion in the Barx2 cDNA from the OVCAR3 ovarian cancer cell line is at the exon 1/exon 2 boundary, which predicts a stop codon.

### EXAMPLE 5

### Identification of a Region of Frequent Loss of Heterozygosity in Colorectal Cancer

### Summary

Seven polymorphic microsatellite repeat loci were analysed by PCR between D11S897 and D11S969 in 50 colorectal tumors. Two distinct regions of loss were detected, suggesting possible sites for genes involved in colorectal neoplasia: a large centromeric region between D11S897 and D11S925 and a telomeric 4.9-Mb region between D11S912 and D11S969. There was no significant correlation with clinicopathological features. This analysis describes a region of LOH in the region 11q23.3-^{24.3} for the first time in colorectal cancer. Barx2 is located close to D11S4131 (LOD score 14 on GB4 radiation hybrid mapping panel) and is located within the interval D11S912-D11S910. This places the Barx2 gene in the centromeric half of the minimal region identified by the LOH studies.

### Introduction

Colorectal carcinogenesis may be explained in terms of activation of oncogenes coupled with inactivation of tumour suppressor genes. These genetic alterations often occur in a certain sequence such as that proposed by Fearon and Vogelstein (1). However, they propose that it is the total accumulation of changes which are responsible for the tumour's progression from adenoma to carcinoma.

The loss of specific chromosomal regions usually involves only one of the two parental chromosomes in normal cells. These allelic losses have been interpreted as evidence that the regions affected contain tumour suppressor genes whose products normally regulate growth and differentiation in a negative way, hence preventing neoplastic development.

At present in colorectal cancer, there are known regions of LOH in association with tumor suppressor genes on chromosomes 5q (APC) (2), 17p (p53) (3), 5q (MCC) (4) and 18q (DCC) (5) which occur frequently in association with somatic mutation of tumour suppressor genes but many other chromosomes also show areas of allelic loss.

Chromosome 11 was considered a candidate for harbouring tumour-suppressor genes because of cytogenetic analyses on colorectal cancers which have found frequent deletions of the long arm of chromosome 11 (6, 7).

There is evidence (8) that human colon carcinoma cells into which a normal copy of chromosome 11 had been transferred show a reduced tumour growth rate in vivo although there is no suppression of tumorigenicity. This suggests the presence of a gene on chromosome 11 which affects cell growth, although the position of this gene, if it exists, has not yet been determined. There are several candidate tumour suppressor genes on 11p which could be responsible for this effect such as WT1 (11p13) (9), so-called WT2 (11p15.5) (10), p57 (11p15.5) (11), TSG 101 (11p13) (12), and KAI-1 (11p13) (13).

The existing evidence for loss of heterozygosity (LOH) on chromosome 11q in-colorectal cancer is conflicting. Gustafson et al (14) analysed 101 samples for allelic loss at the DRD2 gene located at 11q22-23 where they found a significant association of LOH of this region with losses on chromosome 14. However; Keldysh et al (15) were able to map LOH to 11q22-23 and correlate the deletions to clinicopathological characteristics. Deletions of this region showed a trend to significance in association with rectal rather than colonic sites and with well differentiated tumours.

A region of LOH has recently been discovered (16-18) at 11q23.3-q24.3 in epithelial ovarian cancer which is associated with poor prognosis. In the light of this new region of loss of heterozygosity which has been mapped and linked to survival in ovarian cancer, the regions 11q22-q23.3 and 11q23.3-24.3 were examined in DNA derived from colorectal tumour samples. The aim of this study was to map the above region using oligonucleotide primers in a series of blood/tumour pairs from a population of patients with colorectal cancer.

### Materials and Methods

### Patient Population and Tumour Samples

Fresh primary colorectal tumour tissue from 50 patients was obtained at the time of operation and tumour was microdissected from surrounding normal tissue. DNA was extracted according to standard methods as previously described (19). Patient characteristics are outlined in Table 1. Patients are on continuing follow-up which ranges up to 2492 days.

TABLE 1
Clinicopathological characteristics of the study cohort

| Patients' Age | |
|---|---|
| Mean age | 67.3 years |
| Median | 68 years |
| Standard deviation | 10.31 |
| Sex | |
| Female | 23 |
| Male | 24 |
| Unknown | 3 |
| Anatomical location | |
| Ascending | 9 |
| Transverse | 4 |
| Descending | 2 |
| Sigmoid | 19 |
| Rectum | 12 |
| Unknown | 4 |
| Dukes' Stage | |
| A | 1 |
| B | 26 |
| C1 | 19 |
| Unknown | 4 |
| Differentiation | |
| Well | 3 |
| Moderate | 33 |
| Poor | 9 |
| Unknown | 5 |
| Vascular Invasion | |
| No | 41 |
| Yes | 3 |
| Unknown | 6 |
| Perineural Infiltration | |
| No | 42 |
| Yes | 2 |
| Unknown | 6 |
| Mucin Production | |
| No | 35 |
| Yes | 9 |
| Unknown | 6 |

### LOH Analysis

DNA samples were analysed as normal/tumour pairs by PCR using primers for the CA repeat polymorphic microsatellites in the region 11q23 to 11q24.3 covering a physical distance of 491 cRay. Primer sequences were obtained from the Genome Data Base and the location determined from the radiation hybrid map produced by James et al (20). Each primer was optimised with HeLa DNA to determine the ideal PCR reaction conditions.

The PCR products were resolved by electrophoresis on a 6% denaturing urea/polyacrylamide gel, passively transferred to Hybond nylon membrane and exposed to ultra-violet light to cross-link the DNA to the filter. The products were probed with g-32P end-labelled (CA)35 oligonucleotide and exposed to film.

The autoradiographs were analysed by visual reporting and by computer densitometric analysis. Autoradiographic data was acquired using GDS 7500 Gel Documentation System (UVP) and analysed using GelBase Pro software V3.11 (Synoptics Ltd). Each pair of samples was assigned to one of four groups; heterozygous with LOH, heterozygous without LOH, uninformative (homozygous) or not determinable. The relative ratio of alleles was determined, normalised and compared. Where the tumour allele ratio differed from the normal allele by 30% or more (r<0.7) LOH was assigned as previously described (21).

### Statistical Analysis

Fisher's exact test was used to look for associations between LOH regions and clinicopathological parameters.

### Results

Clinical and pathological characteristics of the cohort are outlined in Table 1.

LOH was detected somewhere on chromosome 11 in 47 (94%) of 50 tumours in this series.

FIG. 1a shows the regions of LOH in all of the tumour samples. A centromeric region of loss was defined by D11S897 and D11S925. There is a secure area of loss bordered by these two markers in 7 tumours. This area of loss may also be present in a further 26 tumours where only one or other of the markers is unambiguously deleted. This suggests a maximum LOH rate for this centromeric region of 66%. In order to confirm this region, a larger number of microsatellites should be used to accurately map this area.

A distal region defined by the 3 telomeric markers (D11S912, D 11S1320), D11S969) was deleted in 35 out of 50 tumours (70%). Of tumours with this deletion, several (11 out of 35, 31%) showed loss of D11S1320 only with retention of the adjacent centromeric and telomeric markers. D11S1320 demonstrated LOH in 30 out of 50 (60%) of cases. Clear examples of LOH selectively at this locus are shown in FIG. 1b.

There was no significant correlation between regional losses and any recorded clinicopathological features (survival, sex, site of tumor, differentiation, vascular invasion, perineural invasion, or mucin production).

### Discussion

The above data maps a region of LOH to 11q23.3-24.3 for the first time in colorectal cancer. Recent evidence for a late-acting tumour suppressor gene in the region 11q23.3-24.3 has been described for ovarian cancer (17) and the data presented here not only confirms the existence of an identical region in colorectal cancer but also reduces the likely region housing a tumour suppressor gene from 8.5 Mb to 4.9 Mb, lying between D11S912 and D11S969. No association with adverse clinicopathological features was noted in this series of colorectal tumors. This is consistent with the findings of Keldysh et al (15) which also did not associate LOH on 11q with adverse clinicopathological features.

A recent study (22) concluded that distal loss on chromosome 11 was not frequent in colorectal tumours. The regions examined were 11q22-q23.1 and 11q25-qterm; in particular their most telomeric marker (D11S969) was lost at a low rate (15%, 3 out of 20 cases). Although methodological differences preclude direct comparison, their analysis concluded that there was no significant LOH at 11q25 in contra-distinction to a parallel series of breast cancer cases reported concurrently by the same authors. Our study in contrast clearly demonstrates a region of LOH lying just centromeric to D11S969 in colorectal cancer. This region contains the Barx2 gene.

Human Barx2 was localised using the GeneBridge 4 Radiation Hybrid mapping panel as supplied by UK Human Gene Mapping Project (HGMP) Resource Centre, Hinxton, Cambridge, CB10 1SB UK. An exon 4 specific PCR for Barx2 was performed on the panel DNAs and the results analysed using the RHyME programme accessed through the UK HGMP Resource Centre web site (http://www.hgmp.mrc.ac.uk).

GeneBridge4 Radiation Hybrid Mapping Results from RHyME:

| Chromosome | FW | Marker 1 | Marker 2 | Theta | Lod |
|---|---|---|---|---|---|
| 11 | GM | F8R8 | AFMb002vd1 | 0.156 | 14.379 |
| 11 | GM | F8R8 | AFM321xe9 | 0.158 | 14.014 |
| 11 | GM | F8R8 | AFM248wf5 | 0.165 | 13.373 |
| 11 | GM | F8R8 | AFM200vg5 | 0.210 | 12.147 |
| 11 | GM | F8R8 | AFM324zh9 | 0.217 | 11.683 |

Marker 1 = HBARX2 (PCR used was HBARX2F8/HBARX2R8)
Marker 2 = AFMb002vd1 = D11S4131 = SHGC-20715

Top lod score is with the marker D11S4131 (also known as AFMb002vd1). Therefore, from this analysis, Barx2 is most likely to be located closest to the marker D11S4131, which from Gene Map99 (http://www.ncbi.nlm.nih.gov/genemap) is located between D11S912 and D11S910.

### References for Example 5

1. Fearon, E. R. and Vogelstein, B. A genetic model for colorectal tumorigenesis. (1990) Cell 61, 759-767.

2. Bodmer, W. F., Bailey, C. J., Bodmer, J., Bussey, H. J., Ellis, A., Gorman, P., Lucibello, F. C., Murday, V. A., Rider, S. H., and Scambler, P. Localisation of a gene for familial adenomatous polyposis on chromosome 5. (1987) Nature 328, 614-616.

3. Lane, D. P. and Crawford, L. V. T antigen is bound to host protein in SV40-transformed cells. (1979) Nature 278, 261-263.

4. Kinzler, K. W., Nilbert, M. C., Vogelstein, B., Bryan, T. M., Levy, D. B., Smith, K. J., Preisinger, A. C., Hamilton, S. R., Hedge, P., Markham, A., Carlson, M., Joslyn, G., Groden, J., White, R., Miki, Y., Miyoshi, Y., Nishisho, I., and Nakamura, Y. Identification of a gene located at chromosome 5q21 that is mutated in colorectal cancers. (1991) Science 251, 1366-1370.

5. Fearon, E. R., Cho, K. R., Nigro, J. M., Kern, S. E., Simons, J. W., Ruppert, J. M., Hamilton, S. R., Preisinger, A. C., Thomas, G., Kinzler, K. W., and Vogelstein, B. Identification of a chromosome 18q gene that is altered in colorectal cancers. (1990) Science 247, 49-56.

6. Mulleris, M., Salmon, R. J., and Dutrillaux, B. Cytogenetics of colorectal adenocarcinomas. (1990) Cytogenet Cell Genet. 46, 143-156.

7. Konstantinova, L. N., Fleischman, E. W., Knisch, V. I., Perevozchikov, A. G., and Kopnin, B. P. Karyotype pecularities of human colorectal adenocarcinomas. (1991) Hum Genet. 86, 491-496.

8. Tanaka, K., Oshimura, M., Kikuchi, R., Seki, M., Hayashi, T., and Miyaki, M. Suppression of tumorigenicity in human colon carcinoma cells by introduction of normal chromosome 5 or 18. (1991) Nature 349, 340-2.

9. Call, K. M., Glaser, T., Ito, C. Y., Buckler, A. J., Pelletier, J., Haber, D. A., Rose, E. A., Kral, A., Yeger, H., Lewis, W. H., Jones, C., and Housman, D. E. Isolation and characterization of a zinc finger polypeptide gene at the human chromosome 11 Wilms' tumor locus. (1990) Cell 60, 509-520.

10. Dowdy, S. F., Fasching, C. L., Araujo, D., Lai, K. M., Livanos, E., Weissman, B. E., and Stanbridge, E. J. Suppression of tumorigenicity in Wilms tumor by the p15.5-p14 region of chromosome 11. (1991) Science 254, 293-295.

11. Lee, M. H., Reynisdottir, I., and Massague, J. Cloning of p57KIP2, a cyclin dependent kinase inhibitor with unique domain structure and tissue distribution. (1995) Genes Dev. 9, 639-649.

12. Li, L. and Cohen, S. N. tsg101: A novel tumor susceptibility gene isolated by controlled homozygous functional knockout of allelic loci in mammalian cells. (1996) Cell 85, 319-329.

13. Dong, J. T., Lamb, P. W., Rinkerschaeffer, C. W., Vukanovic, J., Ichikawa, T., Isaacs, J. T., and Barrett, J. C. Kail, a metastasis suppressor gene for prostate-cancer on human-chromosome 11p11.2. (1995) Science 268, 884-886.

14. Gustafson, C. E., Young, J., Leggett, B., Searle, J., and Chenevix-Trench, G. Loss of heterozygosity on the long arm of chromosome 11 in colorectal tumours. (1994) Br J Cancer 70, 395-397.

15. Keldysh, P. L, Dragani, T. A., Fleischman, E. W., Konstantinova, L. N., Perevoschikov, A. G., Pierotti, M. A., Della-Porta, G., and Kopnin, B. P. 11q Deletions in human colorectal carcinomas: Cytogenetics and restriction fragment length polymorphism analysis. (1993) Genes Chromosomes Cancer 6, 45-50.

16. Gabra, H., Taylor, L., Cohen, B. B., Lessels, A., Eccles, D. M., Leonard, R. C. F., Smyth, J. F., and Steel, C. M. Chromosome 11 allele imbalance and clinicopathological correlates in ovarian tumours. (1995) Br J Cancer 72, 367-375.

17. Gabra, H., Watson, J. E. V., Taylor, K. J., MacKay, J., Leonard, R. C. F., Steel, C. M., Porteous, D. J., and Smyth, J. F. Definition and refinement of a region of loss heterozygosity at 11q23.3-q24.3 in epithelial ovarian-cancer associated with poor-prognosis. (1996a) Cancer Res. 56, 950-954.

18. Gabra, H., Watson-JEV, Eccles, D. M., Taylor, L., Taylor, K. J., Cohen, B. B., Leonard-RCF, Porteous, D. J., Smyth, J. F., and Steel, C. M. A statistical analysis of chromosome 11 and 17 loss of heterozygosity in epithelial ovarian cancer. (1996b) Int J Oncol. 8, 625-631.

19. Ashton-Rickardt, P. G., Dunlop, M. G., Nakamura, Y., Morris, R. G., Purdie, C. A., Steel, C. M., Evans, H. J., Bird, C. C., and Wyllie, A. H. High frequency of APC loss in sporadic colorectal carcinoma due to breaks clustered in 5q21-22. (1989) Oncogene 4, 1169-1174.

20. James, M. R., Richard III, C. W., Schott, J.-J., Youstry, C., Clark, K., Bell, J., Terwilliger, J. D., Hazan, J., Dubay, C., Vignal, A., Agrapart, M., Imai, T., Nakamura, Y., Polymeropoulos, M., Weissenbach, J., Cox, D. R., and Lathrop, G. M. A radiation hybrid map of 506 STS markers spanning human chromosome 11. (1994) Nature Genet. 8, 70-76.

21. Hampton, G. M., Mannermaa, A., Winquist, R., Alavaikko, M., Blanco, G., Taskinen, P. J., Kiviniemi, H., Newsham, I., Cavenee, W. K., and Evans, G. A. Loss of Heterozygosity in sporadic human breast carcinoma: a common region between 11q22 and 11q23.3. (1994) Cancer Res. 54, 4586-4589.

22. Koreth, J., Bakkenist, C. J., and McGee-JO'D. Allelic deletions at chromosome 11q22-q23.1 and 11q25-qterm are frequent in sporadic breast but not colorectal cancers. (1997) Oncogene 14, 431-437.

### EXAMPLE 6

### Analysis of Human Barx2 PAC Clones

PAC clones corresponding to BARX2 were isolated from a human PAC library obtained from the Resource Center/Primary Database (RZPD) of the German Human Genome Project at the Max-Planck-Institute for Molecular Genetics (former Reference Library Database), Heubnerweg 6, 14059 Berlin-Charlottenburg, Germany (WWW: http://www.rzpd.de).

Filters from library 709 (see below) were hybridised with a radioactively labelled probe for Barx2. The template for the probe labelling reaction was a full length coding region PCR product generated by HBARX2F1/HBARX2R3 RT-PCR of human Barx2. Identified clones were then requested from and supplied by RZPD.

The human Barx2 PAC clones are as follows:

Details are given of library filter, co-ordinates and clone identification. Library information and general information pertaining to RZPD are also included.

From RZPD (library no. 709 (RPCI6); Human PAC segment library)

### CLONES

BARX2 PAC1:

Filter 108-1-230

Co-ordinates: 209 10, 280 7

Spotted clone: LLNLP709O0720Q3

Picked clone: LLNLP709O0720Q2

BARX2 PAC2:

Filter 108-1-230

Co-ordinates: 124 127, 123 129

Spotted clone: LLNLP709G2466Q3

Picked clone: LLNLP709G2466Q2

BARX2 PAC3:

Filter 108-1-230

Co-ordinates: 172 133, 172 131

Spotted clone: LLNLP709F1436Q3

Picked clone: LLNLP709F1436Q2

BARX2 PAC4:

Filter 108-2-210

Co-ordinates: 55 159, 52 160

Spotted clone: LLNLP709A1497Q3

Picked clone: LLNLP709A1497Q2

BARX2 PAC5:

Filter 108-1-121

Co-ordinates: 151 230, 151 227

Spotted clone: LLNLP709C18219Q3

Picked clone: LLNLP709C18219Q2

RZPD Human PAC Library Information:

LibNo: 709

LibINFO

Name: RPCI6 Human PAC * Filter per set: 4 * Sets in stock: 0

Creator: Pieter de Jong

Description: Human Female PAC Library, see also

http://bacpac.med.buffalo.edu for more information

Condition: Use of the library should be acknowledged by library name (RPCI 6), originating institute (Roswell Park Cancer Institute) and the names of the creators

Condition: Clones from this library have also to be named in publications and database submissions using the following schema:

library name (e.g. RPCI-1 or RPCI-6) followed by plate number (1-n), row character (A-P) and column (1-24)

Library Information for Library No.: 709

Administrative Information

RZPD: Number 709

Name: Human PAC segment

Shortname: RPCI6

Prefix LLNLP

Library type: PAC

Current copy: Q2

384-well-plate

1-240

240 plates (⁻92160 clones)

Experiment Number/Spot Pattern(s) 108: STANDARD 5×5 dup

Filter spotted since March 1997: 256

Condition

Use of the library should be acknowledged by library name (RPCI 6), originating institute (Roswell Park Cancer Institute) and the names of the creators

Clones from this library have also to be named in publications and database submissions using the following schema: library name (e.g. RPCI-1 or RPCI-6) followed by plate number (1-n), row character (A-P) and column (1-24)

The library has a coverage of approx. 4

Creator Information

Dr. Pieter de Jong (Email: pieter@dejong.med.buffalo.edu)

Source Information

Organism: Human (Homo sapiens)

Sex: female

Cloning Information

Vector: pPAC4 (PAC)

Host: E. coli DH10B (DH10B)

Insert size (kb) 135

Empty Vector (%): 2

Picking Information

Antibiotic used for growth: kanamycin

Growth Temp. (° C.): 37

The extent of Barx2 gene represented in RZPD Barx2 PAC Clones was determined as follows.

PAC clone DNA was isolated from each of the Barx2 clones and analysed for Barx2 gene content by genomic PCR. PCR products were then separated by size through agarose containing ethidium bromide and visualised under ultra-violet light following standard gel electrophoresis protocols.

PCRs:

| Exon | PCR Primer Reaction | Product size |
|---|---|---|
| EXON1: | HBARX2F11/HBARX2R5 | 244 bp |
| EXON2: | HBARX2F6/HBARX2R6 | 426 bp |
| EXON3: | HBARX2F10/HBARX2R10 | 285 bp |
| EXON4: | HBARX2F8/HBARX2R8 | 349 bp |

| BARX2 PAC | Exon1 | Exon2 | Exon3 | Exon4 |
|---|---|---|---|---|
| PAC1 | + | + | + | + |
| PAC2 | − | + | + | + |
| PAC3 | − | + | + | + |
| PAC4 | ? | ? | ? | ? |
| PAC5 | + | − | − | − |
| PAC12583 | + | + | + | + |

Results (+ = present; − = absent in PAC clone; ? = ambiguous result)
PAC12583 was obtained from another source.

### EXAMPLE 7

### Fluorescent in situ Hybridisation (FISH) Localises Barx2 to 11q24

FISH was performed using a human Barx2 PAC clone (PAC12583) as the probe. Probes were biotinylated by nick translation using a commercial kit (Boehringer Mannheim). 1 μg of DNA was added to 4 μl translation mix and the mixture made up to 20 μl with distilled water. This was incubated at 15° C. for 90 min before being stopped by addition of 1 μl 0.5M EDTA and heating to 65° C. for 10 min. Unincorporated nucleotides were removed by gel filtration on a Sephadex G50 spin column, and the mixture eluted in 50 μl of TE buffer. Quality was assessed in a standard dot spot assay, and it was determined that 5 μl probe was required per slide.

Slides of OAW42 cell line metaphases were prepared by incubation in a 0.01 mg/ml solution in SSC buffer for 1 hour at 37° C. before being dehydrated through graded alcohols and dried under vacuum. Denaturation of slides was carried out in a 50% formamide/20% SCC soln. at 70% for 3 min before dehydrating through 70% -100% ethanol.

Biotinylated Barx2 PAC probe was prepared by adding 5 μl probe to 1 mg Cot-1 DNA and 5 mg sonicated salmon sperm DNA per slide to an Epperndorf tube, adding an equal volume of ethanol and drying under vacuum. This was then re-suspended in 15 μl per slide FITC labelled chromosome 11 paint and the resulting solution denatured at 70° C. for 10 min before being applied to the slide. After overnight incubation at 43° C. the coverslips were removed and the slides washed in 2×SSC/50% formamide 4 times over 15 min at 43° C., followed by similar washes in 2×SSC, and finally washed in 0.1×SSC at 60° C. before being placed in 4×SSC/Tween 20 buffer. The probe was visualised by incubating for 30 min in 1:200 avidin/TR (Vector Labs) followed by sinilar incubations in 1:200 biotinylated anti-avidin (Vector Labs), and another incubation in avidin/TR. After mounting in Vectashield (Vector Labs) containing 5 μl of a 20 μg/ml DAPI soln per 250 μl, the slides were viewed under a fluorescence microscope equipped with a camera to allow images to be edited before printing out.

### EXAMPLE 8

### Human Barx2 Genomic PCR

Detailed are PCR primers to permit amplification of exons from the human BARX2 gene from genomic DNA (see FIG. 2).

| | | Nucleo- | |
|---|---|---|---|
| tides | | | |
| | | (AF031924 | |
| | | Number- | Ex- |
| Primer Name | Primer sequence | ing) | on |
| HBARX2 F4: | 5′-CGGGCGAAGAGATCTACCCG-3′ | 1035-1054 | 4 |
| HBARX2 F5: | 5′-GAGCTCGCGGCCAGCTCAAA-3′ | 123-142 | 1 |
| | | (5′UTR) | |
| HBARX2 F6: | 5′-CAGGTCCTGGCCTGCTTCCC-3′ | intron 1 | |
| HBARX2 F7: | S′-TGTCAGCAGGATCCCATCTC-3′ | intron 2 | |
| HBARX2 F8: | 5′-TGGAGGGAAGGAATTATTTC-3′ | intron 3 | |
| HBARX2 R4: | 5′-ATGCTAGGATATAGGGCTTG-3′ | 1259-1240 | 4 |
| | | (3′UTR) | |
| HBARX2 R5: | S′-TACACGGACGTGAAAGCTAC-3′ | intron 1 | |
| HBARX2 R6: | 5′-CCCACAATGGGAGCAAGTCT-3′ | intron 2 | |
| HBARX2 R7: | S′-ATACAAGTCAGTACTCATTG-3′ | intron 3 | |
| HBARX2 R8: | 5′-AGTCTCCCTCTTCCCTCAAA-3′ | 965-946 | 4 |

Exon 1 Primer: P11 which can substitute for P5 in
Exon 1 PCR

| | | |
|---|---|---|
| HBARX2F11: | 5′-TCACCATGCACTGCCACG-3′ | nucs: |
| | | 92-109 |

Alternative Exon 2 (genomic) PCR Pimers: HBARX2F9/HBARX2R9 F9/R9-to PCR exon 2 from the intron/exon boundaries. First 4 bases of each oligo are from donor/acceptor splice sites.

| | |
|---|---|
| HBARX2F9: | 5′-CCAGGCTCCCCTTCCCTGCGGGCA-3′ |
| HBARX2R9: | 5′-TCACCTGTCTGGGGTTGACAAATA-3′ |

Product size from genomic DNA: 301 bp(exon2)+4 bp(intron1)+4 bp(intron2)=309 bp total

Alternative Exon3 Primers (based on new B. Nelkin sequence)

| | |
|---|---|
| HBARX2 F10: | 5′-TCCTGCTGCCTCCCATTCTG-3′ |
| HBARX2 R10: | 5′-CAACAGCTTCCCCGCAAGCC-3′ |

product sizes:

genomic DNA: 285 bp (intron 2: 54 bp +exon 3: 85 bp +intron 3: 146 bp)

Human BARX2 Primer combinations for Genomic PCR:

| Exon | PCR Primer Combination | Product size |
|---|---|---|
| EXON1: | HBARX2F5/HBARX2R5 | 213 bp |
| EXON1: | HBARX2F11/HBARX2R5 | 244 bp |
| EXON2: | HBARX2F6/HBARX2R6 | 426 bp |
| EXON2: | HBARX2F9/HBARX2R9 | 309 bp |
| EXON3: | HBARX2F7/HBARX2R7 | 256 bp |
| EXON3: | HBARX2F10/HBARX2R10 | 285 bp |
| EXON4: | HBARX2F8/HBARX2R8 | 349 bp |

Human Barx2 sequence of genomic PCR products is shown in FIG. 7. Sequences corresponding to primers are underlined.

The following gives details of genomic SSCPE primers.

In order to facilitate detection of mutations in the human BARX2 gene from patient and cell line material by single strand conformation polymorphism electrophoresis (SSCPE), primers have been designed to yield overlapping products following PCR of genomic DNA corresponding to each exon of human BARX2. The combinations of primers for each exon and the predicted product size are given.

| | | Nucleotide |
|---|---|---|
| | | numbering |
| | | from |
| Primer name | Sequence | AF031924 |
| Exon1: | | |
| HBARX2 R11: | 5′-ACACGGAGTAGAGGGAAAGT-3′ | 236-217 |
| HBARX2 F12: | 5′-ATGATCGACGAGATCCTCTC-3′ | 170-189 |
| Exon2: | | |
| HBARX2 R12: | 5′-AGGACAGTGCCTGGGCGATT-3′ | 389-370 |
| HBARX2 F13: | 5′-TCATCTCCCACCTGGTCCCT-3′ | 339-358 |
| HBARX2 R13: | 5′-CTCGGTGAAGATGGTGCGAC-3′ | 520-501 |
| HBARX2 F14: | 5′-CGAGTCAGAGACGGAACACC-3′ | 451-470 |
| Exon3: | | |
| HBARX2 R14: | 5′-TCTTCCATTTCATCCTGCGA-3′ | 662-643 |
| HBARX2 F15: | 5′-TCAGTCTCTGGGACTCACTC-3′ | 595-614 |
| HBARX2 R15: | 5′-CAAACTGCCAAATGGTCCGG-3′ | intron 3 |

(unpublished intronic sequence obtained from Barry
Nelkin)

| | | |
|---|---|---|
| Exon4: | | |
| HBARX2 R16: | 5′-CTGTTCATCTTCTCTTCAGC-3′ | 771-752 |
| HBARX2 F16: | 5′-GAACTCCATCCCCACATCAG-3′ | 721-740 |
| HBARX2 R17: | 5′-TGGTGGCTCTGCCATCTCTA-3′ | 880-861 |
| HBARX2 F17: | 5′-AGGAGGAGCTCTGTGAAGCA-3′ | 813-832 |

Primer Combinations for Genomic PCR and SSCPE:

| Primers | Product Size | |
|---|---|---|
| Exon1: | | |
| F11/R11 | 145 bp | |
| F1/R5 | 186 bp | 68 bp overlap with F11/R11 |
| Exon2: | | |
| F6/R12 | 165 bp | |
| F13/R13 | 182 bp | 51 bp overlap with F6/R12 |
| F14/R6 | 202 bp | 70 bp overlap with F13/R13 |
| Exon3: | | |
| F10/R14 | 134 bp | |
| F15/R15 | 167 bp | 68 bp overlap with F10/R14 |
| Exon4: | | |
| F8/R16 | 155 bp | |
| F16/R17 | 160 bp | 51 bp overlap withF8/R16 |
| F17/R8 | 153 bp | 68 bp overlap with F16/R17 |

### EXAMPLE 9

### Barx2 Alters Platinum Sensitivity in Cell Lines

Platinum chemotherapy is the mainstay of treatment of ovarian cancer, with high rates of response. Treatment failure is invariably associated with the development of platinum resistance, and understanding mechanisms of this process will potentially lead to improvements in treatment.

In FIG. 4, the 5′ end (F1R1) and the 3′ end (F3R3) of the BarX2 transcript are amplified by RT-PCR using the primers given. PEO1, PEO4, PEO6 and PEOCDDP are four cell lines derived from a patient with ovarian cancer. PEO1, taken from the patient whilst she was platinum sensitive clearly demonstrates expression of both the 5′ end (F1R1) and the 3′ end (F3R3) of the BARX2 transcript. A cell line developed by exposing PEO1 to cisplatinum in vitro resulted in the PEO1 CDDP cell line which is 18 fold resistant to cisplatin relative to PEO1. Another cell line PEO4 was derived from the same patient when she relapsed again after treatment with high dose cisplatin chemotherapy in vivo. This cell line is approx four-fold resistant relative to PEO1.

In both PEO1CDDP and PEO4, it can be seen that the F1R1 5′ transcript is not detected by RT-PCR, while the 3′ transcript (F3R3) is. A common factor between these two lines is cisplatinum resistance, and further functional analysis was therefore warranted.

The table summarises the Barx2 RT-PCR results on various cell lines.

BARX2

| Cell Line | F1/R1 | F2/R2 | F3/R3 | F1/R3 |
|---|---|---|---|---|
| OVCAR3 | + | + | + | + |
| OVCAR5 | + | + | + | + |
| PEO1 | + | + | + | + |
| PEO1CDDP | − | − | + | − |
| PEO4 | − | − | − | +/− |
| PEO6 | − | + | + | + |
| PEO16 | + | + | + | + |
| SW626 | − | + | + | + |
| A2780 | − | − | + | +/− |
| OAW42 | − | − | − | − |

We tested whether the introduction of Barx2 into PEO1CDDP altered its sensitivity to cisplatinum. 25000 cells were plated. Two days later, varying concentrations of cisplatinum were applied for 3 days, and then removed. Refeeding of the cells was performed every three days until day 13 with cell counts performed every three days.

CH1.1 is a control PEO1CDDP derivative clone that has been transfected with the pBabe Hygro empty vector (the same vector into which BarX2 was cloned) and behave as the parent line. CB3.6 is a BarX2 transfected growth suppressed clone. Both are exposed to increasing concentrations of cisplatinum.

The result of this experiment is seen particularly at the 1 μM cisplatinum concentration. Compared with no cisplatinum, the control cell line grows to 85% control at 1 μM cisplatinum at day 13. At the same timepoint and concentration of platinum, the BarX2 transfected platinum resistant cell line grows to only 69% of control. This increase in platinum sensitivity (or reduction in resistance) upon BarX2 transfection is consistent with the observed aberrant expression of the 5′ BarX2 transcript noted by RT-PCR in the PEO series of cell lines. Whether BarX2 induces sensitivity, reduces resistance, or both is not understood and the underlying mechanism is currently being explored.

It is of note that BARX2 transfected cells grow more slowly than controls, and clinically slower growing cancer cells tend to be less chemosensitive, not more. This result is therefore unexpected from the proliferative state of the cells.

### EXAMPLE 10

### Further Functional Analysis of Barx2

1. Further evidence that Barx2 suppresses growth rate

Over-expression of Barx2 by transfection of pBabeBarx2 suppresses the growth of ovarian cancer cell lines OAW42 (FIG. 17), PEO1 (FIG. 18), and PEO1-CDDP (FIG. 19). See legends for details.

2. Over-expression of Barx2 by transfection suppresses matrigel invasion, haptotactic cellular migration and cellular attachment in the non Barx2 expressing cell line OAW42.

In the ovarian cancer cell line OAW42, we had already shown that expression was undetectable by northern and RT-PCR. Barx2 transfected clonal lines were generated by transfecting pBabeBarx2, and lines expressing non-endogenous Barx2 were obtained (FIG. 20). BX1.2 and 1.7 had the highest relative expression of Barx2. BX1.3 had lower but detectable expression of Barx2. BX1.6 had no detectable expression of Barx2 and genomic PCR of the inserted plasmid showed that it did not contain full length cDNA insert.

Growth suppression of barx2 transfected OAW42 was observed (FIG. 17). There was a direct correlation between levels of non-endogenous expressed Barx2 and extent of functional suppression in the following assays:

Barx2 transfected OAW42 showed suppression of matrigel invasion (see FIG. 21);

Barx2 over-expression in OAW42 suppressed cellular migration in response to a collagen haptotactic signal (see FIG. 22); and

Barx2 over-expression in OAW42 resulted in suppression of cellular adhesion to collagen coated tissue culture plastic (see FIG. 23).

Clonogenic assays for OAW42 cell line transfected with Barx2 or control show no marked difference in clonogenic efficiency suggesting that the observed effects are not due to non-specific toxic effects associated with Barx2 over-expression.

Dunn's multiple comparisons test showed no significant differences between controls and Barx2 transfected OAW42 cells:

| | | median colonies |
|---|---|---|
| Cell Line | no. of plates | at 4 weeks |
| OAW42 | 3 | 131 |
| HYG7.5 | 3 | 84 |
| BX1.7 | 3 | 38 |
| BX1.3 | 3 | 59 |
| BX1.6 | 3 | 53 |

Cell cycle analysis of transfectants reveals that Barx2 over-expression induces late G1/early S phase block (FIG. 24).

3. Barx2 transfection regulates expression of K-cadherin but not E-cadherin.

FIG. 25 shows that K-cadherin (cadherin-6) is not expressed in OAW42 and weakly expressed in A2780. Snc19 is a gene that is closely physically linked to Barx2 (and also not expressed, but induced by demethylation)

OAW42, and pBabeHygro transfected clones (Hy7.5 and Hy7.2) do not express either Barx2 or K-cadherin. BX1.2 and BX1.7 are Barx2 transfected OAW42 clones with the highest barx2 expression level (see FIG. 20). BX1.3 showed barely detectable k-cadherin and Barx2 expression, and BX1.6 showed no expression of either Bar2 or K-cadherin. It is interesting that the functional assays described for OAW42 above show a perfect inverse correlation for the levels of Barx2 and K-cadherin (matrigel invasion, transwell migration and cellular adhesion).

FIG. 26 shows that in BX1.2 and BX1.7 Barx2 expression is directly correlated with K-cadherin expression (CDH6) by RT-PCR.

RT-PCR analysis of E-cadherin reveals that almost all cell lines express E-cadherin abundantly (including OAW42) (data not shown).

OAW42 barx2 transfectants show no alterations for E-cadherin or gamma actin (data not shown) suggesting that Barx2 is not a regulator of E-cadherin, but does specifically regulate K-cadherin in OAW42.

4. Transfection of ovarian cancer cell line A2780 with dominant negative p53 construct is associated with silencing of Barx2 and K-cadherin.

FIG. 27 shows the effect of transfection of a dominant negative mutant p53 of A2780 (a2780mpo53) on Barx2 and CDH6 expression. p53 inactivation is associated with silencing of Barx2 and CDH6 in A2780mpo53. This provides further evidence of a regulatory link between Barx2 and CDH6. AdamTS8, closely linked to Barx2, and located on the same ICI YAC within a few hundred kb physically, shows no such alterations. It therefore is a good positive control for cDNA integrity.

5. Sequential pre- and post-platinum chemotherapy cell lines from 2 patients with ovarian cancer both show down-regulation of Barx2 in association with platinum resistance.

Ovarian cancer cell lines had been established previously in our unit for two patients with ovarian cancer; prior to cisplatin chemotherapy (PEO1 for patient 1, PEO14 for patient 2) and following platinum resistant relapse (PEO4, PEO6 for patient 1; PEO23 for patient 2). For these patients, Barx2 was abundantly expressed prior to cisplatin chemotherapy, and following relapse after platinum therapy, Barx2 was markedly downregulated in the platinum resistant cells. FIG. 28 summarises this information.

6. Transfection of Barx2 reverses acquired cisplatin resistance rather than generally increasing cisplatin sensitivity in PEO1 cell line.

Initial studies demonstrated that introduction of Barx2 into the cisplatin resistant cell line PEO1CDDP reversed platinum resistance in that cell line. Transfection of Barx2 into PEO1 and PEO1CDDP showed that platinum resistance in PEO1CDDP was completely reversed (FIG. 29).

Further analysis of the data showed that sensitivity to cisplatin was not significantly altered in PEO1 as a result of Barx2 transfection across the cisplatin concentration range; however in PEO1CDDP platinum sensitivity was six-fold increased by Barx2 transfection across the same cisplatin range, suggesting that the effect of Barx2 was on acquired resistance rather than intrinsic sensitivity (FIG. 30).

In summary, expression of BARX2 is downregulated/absent in the ovarian cancer cell lines OAW42, A2780, PEO1-CDDP, PEO4, and PEO6. Transfection of BARX2 into OVCAR3, OAW42, PEO1 and PEO1-CDDP suppresses cell growth. In OAW42, this is accompanied by suppression of matrigel invasion, cell migration and adhesion and partial G1/S-phase block. In-vitro (PEO1-CDDP) and in-vivo (PEO6) cisplatin resistant cell lines had reduced expression with evidence of 5′ gene methylation compared with their cisplatin sensitive parent cell line (PEO1), suggesting a mechanism for the observed loss of expression. Transfection of BARX2 into PEO1-CDDP and PEO1 lines demonstrated complete reversal of acquired cisplatin resistance in PEO1-CDDP without significant increase in cisplatin sensitivity in PEO, as demonstrated by 12 day growth inhibition assays. Maximal differences were noted at 1.5 micromolar cisplatin exposure for three days. There were no significant differences by Annexin- V-FACS comparing PEO1 cisplatin sensitive and resistant cell lines at 1.5 or 5 micromolar cisplatin between 1 hr and 40 hr. Furthermore, BARX2 transfection of these cells showed no increase in apoptosis in response to cisplatin suggesting that this was not the mechanism of cell kill. This is consistent with recent thinking for molecular mechanisms of cytotoxicity in clinical cancer, and clonogenic assays are shown in FIG. 35. These data suggest that loss of BARX2 expression may be an important determinant of cisplatin resistance and clinical outcome.

### EXAMPLE 11

### Further Structural Analysis of Barx2

1. Mis-sense mutations have been identified in cell lines.

We have analysed 70 cancer cell lines by DHPLC. The analysis of the lines is ongoing, but we have identified the following mis-sense mutations set out in the Table below. We believe that for PEO14 and 23 (derived from the same patient), that the mutation is in fact germline, with LOH of the normal allele in PEO14 and 23. Expression of Barx2 is strong in PEO14 but markedly reduced in PEO23, suggesting that methylation constitutes a second hit after LOH. That the mis-sense allele was retained after LOH raises the posibility of selective growth advantage ascribable to the Ser→Pro alteration, and this hypothesis is being tested.

| Name | Tumour Type | Exon | Mutation |
|---|---|---|---|
| DX3 | Melanoma | 2 | Heterozygous |
| | | | Missense: Ser→Pro |
| | | | Creates HaeIII site |
| K562 | CML | 2 | Heterozygous |
| | | | Missense: Ala→Pro |
| SKOV3 | Ovarian Cancer | 2 | Heterozygous |
| | | | Missense: Ser→Pro |
| | | | Creates HaeIII site |
| PEO14 | Ovarian Cancer | 2 | Homozygous |
| | | | Missense: Ser→Pro |
| | | | Creates HaeIII site |
| PEO23 | Ovarian Cancer | 2 | Homozygous |
| | | | Missense: Ser→Pro |
| | | | Creates HaeIII site |

The Ser→Pro mutation results from a T to C change at nucleotide 286 with respect to the human Barx2 reference sequence NM003658.2 (updated Dec. 23, 1999). The Ala→Pro mutation results from a G to C change with respect to the human Barx2 reference sequence NM003658.2.

2. Southern analysis reveals that the 5′ end of the barx2 gene is methylated in ovarian cancer cell lines.

FIG. 31 shows identical Southern blots of ovarian cancer cell lines digested either with MspI/BamHI or HpaII/BamHI and probed with a probe encompassing the 5′UTR and first exon of Barx2. 10/19 cell lines exhibit methylation of the 5′ region of barx2 encompassing the distal CpG island and the first exon of Barx2. Methylated cell lines include PEO1, PEO1CDDP, PEO6, PEO14, PEO16, OVCAR3, MDA, HeLa, PEA2, 2780AD and 41M (although OAW42 was not tested in this series).

3. The PEO series of cell lines reveal that down-regulation of Barx2 expression correlates with Barx2 methylation suggesting a mechanism of platinum resistance.

Down-regulation of Barx2 is seen in PEO1CDDP and PEO6 relative to PEO1. The extent of downregulation is proportional to the HpaII methylation signal (FIG. 32). A clonal selection model is proposed to account for the accumulation of methylated, Barx2 downregulated, platinum resistant ovarian cancer clones ( FIG. 33)

4. Analysis of genomic DNA 5′ to barx2 from PAC identifies a CpG island for barx2.

The human PAC clone RZPD BARX2 PAC1 that was shown to contain the complete human BARX2 gene was fragmented by digestion to completion with the restriction endonuclease EcoR1. EcoR1 fragments were then subcloned into the cloning vector pBSIISK+ and transfected into the bacterial host strain JM109.

96 individual bacterial colonies containing EcoR1 inserts were selected and inoculated individually into 96×1.5 ml L-Broth (with ampicillin) in a 96 well culture plate. Bacteria were then grown overnight at 37° C. Clones containing specifically the 5′ end of the BARX2 gene were assayed for by screening 2 μl of each overnight culture sample by PCR with the primers BARX2 F11/BARX2 R5. 9 colonies were shown to be positive in this 96-well screen.

Plasmid DNA was isolated from positive clones and the size of the EcoR1 fragment was estimated by separation on agarose following release of the insert by EcoR1 digestion. The 5′ end of the BARX2 gene (as defined by the BARX2 F11/BARX2 R5 PCR assay) is contained within a 12 kb EcoR1 fragment.

The 12 kb EcoR1 fragment was purified and digested with Mse1 in order to produce smaller fragments that were then subcloned into the vector pGEM-T Easy (Promega) and transfected into the bacterial strain JM109.

96 individual bacterial colonies containing Mse1 inserts were inoculated into 1.5 ml L Broth (with ampicillin) in a 96 well plate and cultures grown overnight. Mse1 fragments corresponding to the 5′ end of the BARX2 gene were then identified in a 96-well PCR of 2 ul of each culture using the primers BARX2 F11/R5. 2 positive clones were identified and the plasmid DNA prepared from them using standard methods. The size of the BARX2 gene 5′ Mse1 fragment that therefore contains the CpG island was estimated to be 700 bp following release of the insert by EcoRI digestion of the plasmid DNA.

5.-Azacytidine demethylation of OAW42 re-expresses Barx2.

OAW42 was exposed to 5-azacytidine for 96 hours at different concentrations. Clear induction of Barx2 expression was observed at 0.5 micromolar azacytidine. This suggests that Barx2 is methylated, and that demethylation in OAW42 results in re-expression of Barx2.

In summary, two mis-sense mutations have been identified in a panel of cancer cell lines. Both are contained within exon 2, 5′ of the homeodomain. The 5′ end of the gene is methylated in a number of cancer cell lines as demonstrated in MspI/HpaII Southern blots, suggesting a possible mechanism for gene silencing.

Transfection of full length BARX2 cDNA into OAW42 confers in-vitro suppression of growth, migration, adhesion to collagen and matrigel invasion. Northern analysis demonstrates that transfected BARX2 is expressed at low levels; overexpression of the gene may be lethal to cells. FACS analysis demonstrates that transfection results in S-phase block. BARX2 transfection completely reverses acquired cisplatin resistance in PEO1-CDDP, with no such effect on the PEO1 sensitive parent line.

### LIST OF ADDITIONAL REFERENCES

Anderson et al (1980) Proc. Natl. Acad. Sci. USA 77, 5399-5403.

Berkner (1992) Curr. Top. Microbiol. Immunol. 158, 39-61.

Berkner et al (1988) BioTechniques 6, 616-629.

Brandyopadhyay and Temin (1984) Mol. Cell. Biol. 4, 749-754.

Breakfield and Geller (1987) Mol. Neurobiol. 1, 337-371.

Brinster et al (1981) Cell 27, 223-231.

Buchschacher and Panganiban (1992) J. Virol. 66, 2731-2739.

Capecchi, M. R. (1989) Science 244, 1288.

Constantini and Lacy (1981) Nature 294, 92-94.

Cotten et al (1990) Proc. Natl. Acad. Sci. USA 87, 4033-4037.

Culver et al (1992) Science 256, 1550-1552.

Curiel et al (1991a) Proc. Natl. Acad. Sci. USA 88, 8850-8854.

Curiel et al (1991b) Hum. Gene Ther. 3, 147-154.

Donehower, L. A. et al (1992) Nature 356, 215.

Erickson, J. et al (1990) Science 249, 527-533.

Felgner et al (1987) Proc. Natl. Acad. Sci. USA 84, 7413-7417.

Fink et al (1992) Hum. Gene Ther. 3, 11-19.

Freese et al (1990) Biochem. Pharmacol. 40, 2189-2199.

Friedman, T. (1991) In Therapy for Genetic Diseases, T. Friedman, ed., Oxford University Press, pp 105-121.

Gorziglia and Kapikian (1992) J. Virol. 66, 4407-4412.

Graham and van der Eb (1973) Virology 52, 456-467.

Hasty, P. K. et al (1991) Nature 350, 243.

Helseth et al (1990) J. Virol. 64, 2416-2420.

Hodgson, J. (1991) Bio/Technology 9, 19-21.

Johnson et al (1992) J. Virol. 66, 2952-2965.

Kaneda et al (1989) J. Biol. Chem. 264, 12126-12129.

Lim et al (1992) Circulation 83, 2007-2011.

Madzak et al (1992) J. Gen. Virol. 73, 1533-1536.

Mann and Baltimore (1985) J. Virol. 54, 401-407.

Margolskee (1992) Curr. Top. Microbiol. Immunol. 158, 67-90.

Miller (1992) Curr. Top. Microbiol. Immunol. 158, 1-24.

Miller et al (1985) Mol. Cell. Biol. 5, 431-437.

Miller et al (1988) J. Virol. 62, 4337-4345.

Mombaerts, P. et al (1992) Cell 68, 869.

Moss (1992) Curr. Top. Microbiol. Immunol. 158, 25-38.

Muzyczka (1992) Curr. Top. Microbiol. Immunol. 158, 97-123.

Nabel (1992) Hum. Gene Ther. 3, 399-410.

Ohi et al (1990) Gene 89, 279-282.

Page et al (1990) J. Virol. 64, 5370-5276.

Pellicer et al (1980) Science 209, 1414-1422.

Petropoulos et al (1992) J. Virol. 66, 3391-3397.

Philpott, K. L. et al (1992) Science 256, 1448.

Quantin et al (1992) Proc. Natl. Acad. Sci. USA 89, 2581-2584.

Rosenfeld et al (1992) Cell 68, 143-155.

Shimada et al (1991) J. Clin. Invest. 88, 1043-1047.

Shinkai, Y. et al (1992) Cell 68, 855.

Snouwaert, J. N. et al (1992) Science 257, 1083.

Sorge et al (1984) Mol. Cell. Biol. 4, 1730-1737.

Stewart et al (1992) Hum. Gene Ther. 3, 267-275.

Stratford-Perricaudet et al (1990) Hum. Gene Ther. 1, 241-256.

Valancius, V. & Smithies, O. (1991) Mol. Cell Biol. 11, 1402.

Wagner et al (1991) Proc. Natl. Acad. Sci. USA 88, 4255-4259.

Wang and Huang (1989) Biochemistry 28, 9508-9514.

Wells, J. A. (1991) Methods in Enzymol. 202, 390-411.

Wilkinson et al (1992) Nucleic Acids Res. 20, 2233-2239.

Wolff et al (1990) Science 247, 1465-1468.

Wolff et al (1991) BioTechniques 11, 474-485.

Wu et at (1989a) Genomics 4, 560-569.

Wu et al (1991) J. Biol. Chem. 266, 14338-14342.

Zenke et al (1990) Proc. Natl. Acad. Sci. USA 87, 3655-3659.

1. A method for determining the susceptibility of a patient to cancer comprising the steps of
(i) obtaining a sample containing nucleic acid from the patient; and
(ii) contacting the said nucleic acid with a nucleic acid which hybridises selectively to the Barx2 gene or a mutant allele thereof, or a nucleic acid which hybridises selectively to Barx2 cDNA, or a mutant allele thereof, or their complement.

2. A method of diagnosing cancer in a patient comprising the steps of
(i) obtaining a sample containing nucleic acid from the patient; and
(ii) contacting the said nucleic acid with a nucleic acid which hybridises selectively to the Barx2 gene, or a mutant allele thereof, or a nucleic acid which hybridises selectively to Barx2 cDNA, or a mutant allele thereof, or their complement.

3. A method of predicting the relative prospects of a particular outcome of a cancer in a patient comprising the steps of
(i) obtaining a sample containing nucleic acid from the patient; and
(ii) contacting the said nucleic acid with a nucleic acid which hybridises selectively to the Barx2 gene, or a mutant allele thereof, or a nucleic acid which hybridises selectively to Barx2 cDNA, or a mutant allele thereof, or their complement.

4. A method according to any one of the preceding claims wherein the cancer is ovarian cancer or colon cancer.

5. A method according to any one of the preceding claims wherein the sample is a sample of the tissue in which cancer is suspected or in which cancer may be or has been found.

6. A method according to any one of the preceding claims wherein the sample is a sample of ovary and the cancer is ovarian cancer.

7. A method according to any one of the preceding claims wherein the nucleic acid which selectively hybridises to the human-derived DNA of said Barx2 gene or the said Barx2 cDNA sequence, or a mutant allele thereof, or their complement, further comprises a detectable label.

8. A method according to any one of the preceding claims wherein the nucleic acid which selectively hybridises as said is single-stranded.

9. A method according to any one of the preceding claims wherein the nucleic acid which selectively hybridises as said has fewer than 10000 base pairs when the nucleic acid is double-stranded or bases when the nucleic acid is single-stranded.

10. A method according to any one of the preceding claims wherein the nucleic acid which selectively hybridises as said has fewer than 1000 base pairs when the nucleic acid is double-stranded or bases when the nucleic acid is single-stranded.

11. A method according to any one of the preceding claims wherein the nucleic acid which hybridises as said has from 10 to 100 base pairs when the nucleic acid is double-stranded or bases when the nucleic acid is single-stranded.

12. A method according to any one of the preceding claims wherein the nucleic acid which hybridises as said has from 15 to 30 base pairs when the nucleic acid is double-stranded or bases when the nucleic acid is single-stranded.

13. A method according to any one of claims 1 to 3 wherein the nucleic acid which hybridises as said comprises a portion of the human-derived DNA of PAC1, or a portion of Barx2 cDNA.

14. A method according to claim 13 wherein the portion is a single-stranded portion.

15. A method according to claim 14 wherein said portion is capable of amplifying a portion of the Barx2 gene or the Barx2 cDNA or mRNA in a nucleic acid amplification reaction.

16. A method for determining the susceptibility of a patient to cancer comprising the steps of
(i) obtaining a sample containing protein derived from the patient; and
(ii) determining the relative amount, or the intracellular location, or physical form, of the Barx2 polypeptide, or the relative activity of, or change in activity of, or altered activity of, the Barx2 polypeptide.

17. A method of diagnosing cancer in a patient comprising the steps of
(i) obtaining a sample containing protein derived from the. patient; and
(ii) determining the relative amount, or the intracellular location, or physical form, of the Barx2 polypeptide, or the relative activity of, or change in activity of, or altered activity of, the Barx2 polypeptide.

18. A method of predicting the relative prospects of a particular outcome of a cancer in a patient comprising the steps of
(i) obtaining a sample containing protein derived from the patient; and
(ii) determining the relative amount, or the intracellular location, or physical form of the Barx2 polypeptide, or the relative activity of, or change in activity of, or altered activity of, the Barx2 polypeptide.

19. A method according to any one of claims 16 to 18 wherein the cancer is ovarian cancer or colon cancer.

20. A method according to any one of claims 16 to 19 wherein the sample is a sample of the tissue in which cancer is suspected or in which cancer may be or has been found.

21. A method according to any one of claims 16 to 20 wherein the sample is a sample of ovary and the cancer is ovarian cancer.

22. A method according to any one of claims 16 to 21 wherein the relevant amount, or intracellular location, of the Barx2 polypeptide is determined using a molecule which selectively binds to Barx2 polypeptide or a natural variant or fragment thereof.

23. A method according to claim 22 wherein the molecule which selectively binds Barx2 polypeptide or a natural variant or fragment thereof is an anti-Barx2 antibody.

24. A method according to any one of claims 16 to 21 wherein the relevant amount, or intracellular location, of the Barx2 polypeptide is determined by assaying or detecting the activity of the Barx2 polypeptide.

25. A method according to claim 22 or claim 23 wherein the molecule which selectively binds to Barx2 comprises a detectable label.

26. Use of a nucleic acid which selectively hybridises to the Barx2 gene, or a mutant allele thereof, or a nucleic acid which hybridises selectively to Barx2 cDNA, or a mutant allele thereof, or their complement, in the manufacture of a reagent for diagnosing cancer.

27. Use of a molecule which selectively binds to Barx2 polypeptide or a natural fragment or variant thereof in the manufacture of a reagent for diagnosing cancer.

28. Use of a nucleic acid as defined in claim 26 in a method of diagnosing cancer.

29. Use of a molecule which selectively binds to Barx2 polypeptide or a natural fragment or variant thereof in a method of diagnosing to cancer.

30. A method of determining loss of heterozygosity in a tissue sample, the method comprising the steps of (i) obtaining a sample containing nucleic acid derived from the tissue and (ii) comparing a microsatellite profile of the said nucleic acid with that of a reference (homozygous) tissue, the microsatellite(s) being chosen by reference to the Barx2 gene.

31. A method of treating cancer comprising the step of administering to the patient a nucleic acid which selectively hybridises to the Barx2 gene or a nucleic acid which hybridises selectively to Barx2 cDNA.

32. A method of treating cancer comprising the step of administering to the patient a nucleic acid which encodes the Barx2 polypeptide or a functional variant or portion or fusion thereof.

33. Use of a nucleic acid as defined in claim 26 in the manufacture of a medicament for treating cancer.

34. A method of treating cancer comprising the step of administering to the patient an effective amount of Barx2 polypeptide or a fragment or variant or fusion thereof to ameliorate the cancer.

35. Use of Barx2 polypeptide or a fragment or variant or fusion thereof in the manufacture of a medicament for treating cancer.

36. A method of treating cancer comprising the step of administering to the patient an effective amount of a compound which inhibits the function of a mutant Barx2 polypeptide found in a tumour cell, or which upregulates expression of wild-type Barx2 polypeptide.

37. Use of a compound which inhibits the function of a mutant Barx2 polypeptide, or which upregulates expression of wild-type Barx2 polypeptide, in the manufacture of a medicament for treating cancer.

38. An antibody which reacts with a mutant Barx2 polypeptide of fragment thereof, wherein said mutant Barx2 is a mutant found in a cancer cell.

39. A nucleic acid which selectively hybridises to a nucleic acid encoding a mutant Barx2 polypeptide, wherein said mutant Barx2 is a mutant found in a cancer cell.

40. An antibody according to claim 38 or a nucleic acid according to claim 39 wherein said mutant Barx2 is a mutant found in a cancer cell as disclosed in any of the Examples.

41. A kit of parts comprising a nucleic acid which hybridises selectively to the Barx2 gene or a mutant allele thereof, or a nucleic acid which hybridises selectively to Barx2 cDNA or a mutant allele thereof, and means for detecting a mutation in the Barx2 gene wherein said mutation is a mutation in Barx2 found in a cancer cell.

42. A vector which is capable of expressing the Barx2 polypeptide or a functional fragment or variant or fusion thereof in a mammalian cell.

43. A pharmaceutical composition comprising a gene therapy vector including a nucleic acid which encodes the Barx2 polypeptide or a functional variant or portion or fusion thereof and pharmaceutically acceptable carrier.

44. A pharmaceutical composition comprising a gene therapy vector including a nucleic acid which selectively hybridises to the Barx2 gene, or a mutant allele thereof, or a Barx2 cDNA, or a mutant allele thereof, and a pharmaceutically acceptable carrier.

45. A pharmaceutical composition comprising Barx2 polypeptide or a fragment or variant or fusion thereof, and a pharmaceutically acceptable carrier.

46. A nucleic acid as defined in any one of claims 42 or 44 for use in medicine.

47. Barx2 polypeptide or a fragment or variant or fusion thereof, for use in medicine.

48. A method of identifying a compound which modulates Barx2 function the method comprising contacting Barx2 gene or cDNA or polypeptide or a portion thereof with a test compound and determining its effect.

49. A method of identifying a compound which may be useful in treating cancer the method comprising the steps of claim 48.

50. A method for determining the susceptibility of a patient, to cancer comprising the steps of
(i) obtaining a sample containing the Barx2 gene from the patient;
(ii) determining the degree of methylation of the Barx2 gene;
(iii) comparing the level of methylation of the Barx2 gene from the patient sample with the level of methylation in a non-tumorous sample; and
(iv) if the patient sample has a higher degree of methylation of the Barx2 gene compared to the non-tumorous sample this is indicative of susceptibility to cancer.

51. A method of diagnosing cancer in a patient comprising the steps of
(i) obtaining a sample containing the Barx2 gene from the patient;
(ii) determining the degree of methylation of the Barx2 gene;
(iii) comparing the level of methylation of the Barx2 gene from the patient sample with the level of methylation in a non-tumorous sample; and
(iv) if the patient sample has a higher degree of methylation of the Barx2 gene compared to the non-tumorous sample this is indicative of cancer.

52. A method of predicting the relative prospect of a particular outcome of a cancer patient comprising the steps of
(i) obtaining a sample containing the Barx2 gene from the patient;
(ii) determining the degree of methylation of the Barx2 gene;
(iii) comparing the level of methylation of the Barx2 gene from the patient sample with the level of methylation in a non-tumorous sample; and
(iv) if the patient sample has a higher degree of methylation of the Barx2 gene compared to the non-tumorous sample this is indicative of a lower chance of a successful outcome.

53. A method according to any one of claims 50 to 52 wherein methylation of the Barx2 gene promoter is analysed.

54. A method of determining whether a tumour cell is likely to be sensitive to platinum chemotherapy comprising the steps of
(i) obtaining a tumour cell;
(ii) determining the level of expression of Barx2 in the cell or the degree of methylation of the Barx2 gene;
(iii) comparing the level of expression of Barx2 or the degree of methylation of the Barx2 gene in the tumour cell with the level of expression or degree of methylation in a non-tumour cell; and
(iv) if the tumour cell has increased expression of Barx2 or a decreased degree of methylation of the Barx2 gene the cell is likely to be sensitive to platinum chemotherapy.

55. Any novel method of diagnosis, prognosis or treatment of cancer as herein disclosed.


SEQUENCE LISTING (54 sequences)

SEQ ID NO: 1 - DNA, 20 bp, Artificial Sequence
  Description of Artificial SequencePCR primer
atgatcgacgagatcctctc

SEQ ID NO: 2 - DNA, 20 bp, Artificial Sequence
  Description of Artificial SequencePCR primer
caccgaggcggtctctgctg

SEQ ID NO: 3 - DNA, 20 bp, Artificial Sequence
  Description of Artificial SequencePCR primer
tggtatcagaatcgcaggat

SEQ ID NO: 4 - DNA, 20 bp, Artificial Sequence
  Description of Artificial SequencePCR primer
gtgttccgtctctgactcgc

SEQ ID NO: 5 - DNA, 20 bp, Artificial Sequence
  Description of Artificial SequencePCR primer
gcttcctgtccacctttaag

SEQ ID NO: 6 - DNA, 20 bp, Artificial Sequence
  Description of Artificial SequencePCR primer
gcttaatggtgggggttccg

SEQ ID NO: 7 - DNA, 18 bp, Artificial Sequence
  Description of Artificial SequencePCR primer
tcaccatgcactgccacg

SEQ ID NO: 8 - DNA, 20 bp, Artificial Sequence
  Description of Artificial SequencePCR primer
cgggcgaagagatctacccg

SEQ ID NO: 9 - DNA, 20 bp, Artificial Sequence
  Description of Artificial SequencePCR primer
gagctcgcggccagctcaaa

SEQ ID NO: 10 - DNA, 20 bp, Artificial Sequence
  Description of Artificial SequencePCR primer
caggtcctggcctgcttccc

SEQ ID NO: 11 - DNA, 20 bp, Artificial Sequence
  Description of Artificial SequencePCR primer
tgtcagcaggatcccatctc

SEQ ID NO: 12 - DNA, 20 bp, Artificial Sequence
  Description of Artificial SequencePCR primer
tggagggaaggaattatttc

SEQ ID NO: 13 - DNA, 20 bp, Artificial Sequence
  Description of Artificial SequencePCR primer
atgctaggatatagggcttg

SEQ ID NO: 14 - DNA, 20 bp, Artificial Sequence
  Description of Artificial SequencePCR primer
tacacggacgtgaaagctac

SEQ ID NO: 15 - DNA, 20 bp, Artificial Sequence
  Description of Artificial SequencePCR primer
cccacaatgggagcaagtct

SEQ ID NO: 16 - DNA, 20 bp, Artificial Sequence
  Description of Artificial SequencePCR primer
atacaagtcagtactcattg

SEQ ID NO: 17 - DNA, 20 bp, Artificial Sequence
  Description of Artificial SequencePCR primer
agtctccctcttccctcaaa

SEQ ID NO: 18 - DNA, 24 bp, Artificial Sequence
  Description of Artificial SequencePCR primer
ccaggctccccttccctgcgggca

SEQ ID NO: 19 - DNA, 24 bp, Artificial Sequence
  Description of Artificial SequencePCR primer
tcacctgtctggggttgacaaata

SEQ ID NO: 20 - DNA, 20 bp, Artificial Sequence
  Description of Artificial SequencePCR primer
tcctgctgcctcccattctg

SEQ ID NO: 21 - DNA, 20 bp, Artificial Sequence
  Description of Artificial SequencePCR primer
caacagcttccccgcaagcc

SEQ ID NO: 22 - DNA, 20 bp, Artificial Sequence
  Description of Artificial SequencePCR primer
acacggagtagagggaaagt

SEQ ID NO: 23 - DNA, 20 bp, Artificial Sequence
  Description of Artificial SequencePCR primer
atgatcgacgagatcctctc

SEQ ID NO: 24 - DNA, 20 bp, Artificial Sequence
  Description of Artificial SequencePCR primer
aggacagtgcctgggcgatt

SEQ ID NO: 25 - DNA, 20 bp, Artificial Sequence
  Description of Artificial SequencePCR primer
tcatctcccacctggtccct

SEQ ID NO: 26 - DNA, 20 bp, Artificial Sequence
  Description of Artificial SequencePCR primer
ctcggtgaagatggtgcgac

SEQ ID NO: 27 - DNA, 20 bp, Artificial Sequence
  Description of Artificial SequencePCR primer
cgagtcagagacggaacacc

SEQ ID NO: 28 - DNA, 20 bp, Artificial Sequence
  Description of Artificial SequencePCR primer
tcttccatttcatcctgcga

SEQ ID NO: 29 - DNA, 20 bp, Artificial Sequence
  Description of Artificial SequencePCR primer
tcagtctctgggactcactc

SEQ ID NO: 30 - DNA, 20 bp, Artificial Sequence
  Description of Artificial SequencePCR primer
caaactgccaaatggtccgg

SEQ ID NO: 31 - DNA, 20 bp, Artificial Sequence
  Description of Artificial SequencePCR primer
ctgttcatcttctcttcagc

SEQ ID NO: 32 - DNA, 20 bp, Artificial Sequence
  Description of Artificial SequencePCR primer
gaactccatccccacatcag

SEQ ID NO: 33 - DNA, 20 bp, Artificial Sequence
  Description of Artificial SequencePCR primer
tggtggctctgccatctcta

SEQ ID NO: 34 - DNA, 20 bp, Artificial Sequence
  Description of Artificial SequencePCR primer
aggaggagctctgtgaagca

SEQ ID NO: 35 - DNA, 1109 bp, Homo sapiens
  misc_feature (1): A,C,T or G
ntggncctgctcccggccgaaatggcggccgnccgaattcgattgtgttccgtctctgactcgctgctggctagggcctcgccccctggggcctcagcagagaccgcctcggtgacctggtggcaggacagtgcctgggcgattcccggggtggcagggaccaggtgggagatgacagtgggctggcgggtgatcaccgagaggagcggatatgcccgcagggaaggggagccagaatgcagatcattttaaagtttcaggaaacaggatggaaatcaagtcccgtacaggaatgcaggggcttgggtcgcacgascagcgacgggcacactgastacagggaaagtttctcaaagtaatcgcangtctccttggncaggatctcgtcgatcatnatcactangtgaanttcgcggccgcctgcangtcgaccatatgggaaangctcccancgcgttggntgcataacttgagttttctataggggtcacccaanatagcttggggttattcatggtcatanctgtttcntgtgttnaaatttgntancccgcctcacatttccanacnngnatacgancccggtngcatanngtgtaaaagcctggggtgcctaatgnagtgngctanntcaccatnaantgcntttcgctcacngcccgctttccagtccggaaaacctgttccttgcaagcttgcattanatgaattcnggncaaangcnngggggannaangcngttttggcttattgggncgctccttcngnttcctcggntcaattgaatcgcnngtacccccggtctttccgngttngccgnaangtcgggtncnnannttnatncnanagggngncaaananggtttntcccaaannnttagggggaatnatnnngggcancaaantttttggcaaataggngnancaannngggcntttatcnttaaaaggcgctcnttttcnggnngttnntcncaanggcncctnccccttnagngnnnatantacatanancccgttcnaantctatagngntnctcngnncnncatcnggctttacagannnanntcgnggcttcncccctttatgagacccntgattgtncttt

SEQ ID NO: 36 - protein, 116 aa, Homo sapiens
  VARIANT (46): any amino acid
MIDEILXKEXCDYFEKLSLXSVCPSLXVRPKPLHSCTGLDFHPVSXNFKMICILAPLPCGHIRSSRXSPASPLSSPTWSLPPRESPRHCPATRSPRRSLLRPQGARPXPAASQRRN

SEQ ID NO: 37 - DNA, 1454 bp, Homo sapiens
  CDS (1)..(1452)
  VARIANT (286): any amino acid
aggggcccagnccccgccgtcgcgccagccccgcggccccagcgggccgggcactcgcaaccgcgctcgggcggcggacgcgagtgccggctcaccatgcactgccacgccgagctgaggctgagctcgcccggccagctcaaagcagccaggcggcgctacaagactttcatgatcgacgagatcctctccaaggagacctgcgattactttgagaaactttccctctactccgtgtgcccgtcgctggtcgtgcgacccaagcccctgcattcctgtacgggctccccttccctgcgggcatatccgctcctctcggtgatcacccgccagcccactgtcatctcccacctggtccctgccaccccgggaatcgcccaggcactgtcctgccaccaggtcaccgaggcggtctctgctgaggccccagggggcgaggccctagccagcagcgagtcagagacggaacagcccacgccccgacagaagaagccccgccggagtcgcaccatcttcaccgagctgcagctcatgggcctggagaagaaattccagaagcagaagtatttgtcaaccccagacaggttggacttggctcagtctctgggactcactcagctgcaggtgaagacctggtatcagaatcgcaggatgaaatggaagaaaatggttcttaaaggtggacaggaagcacccacaaaacccaaaggtcgccccaagaagaactccatccccacatcagaagagattgaagctgaagagaagatgaacagccaggcccagggtcaggagcagctggagccctctcaggggcaggaggagctctgtgaagcacaggaaccgaaagcacgtgatgtccccttagagatggcagagccaccagacccgccccaggagttgccaataccctcttcggaacccccaccattaagctaagtaaaacccttttgagggaagagggagactggggagaagggaaaagagagaaggcagggagagtagggagagaaaaccttccagcagcccagtaaactgcgggcgaagagatctacccgtctccctccctcccacagttaccattggccttgtcatcgcaagcatttgacaaagacttgcttgtcttgggcctgtcacctcctgaaaggctgctttagctgtggatgcccttgattaagggagagagcgcctaggagctgcctgccccagctggggtgacggctgtagggctgggtctatgttgcaagccctatatcctagcatgcagtggaaagtgcttagctctctccctcctgacctctgggcagccagtcatcaaagcagagagacgtggcggcatgtgggcagcatgcccaggttccttgctgactcagcacttatttctgtagttttaaaaaagaatttaatgtttttggttgtatttttttgggggggtgagggtgggcaaaaacatggga

SEQ ID NO: 38 - protein, 311 aa, Homo sapiens
  VARIANT (4): any amino acid
RGPXPAVAPAPRPQRAGHSQPRSGGGRECRLTMHCHAELRLSSPGQLKAARRRYKTFMIDEILSKETCDYFEKLSLYSVCPSLVVRPKPLHSCTGSPSLRAYPLLSVITRQPTVISHLVPATPGIAQALSCHQVTEAVSAEAPGGEALASSESETEQPTPRQKKPRRSRTIFTELQLMGLEKKFQKQKYLSTPDRLDLAQSLGLTQLQVKTWYQNRRMKWKKMVLKGGQEAPTKPKGRPKKNSIPTSEEIEAEEKMNSQAQGQEQLEPSQGQEELCEAQEPKARDVPLEMAEPPDPPQELPIPSSEPPPLS

SEQ ID NO: 39 - protein, 4 aa, Homo sapiens
VKPF

SEQ ID NO: 40 - protein, 16 aa, Homo sapiens
GKRETGEKGKERRQGE

SEQ ID NO: 41 - protein, 55 aa, Homo sapiens
GEKTFQQPSKLRAKRSTRLPPSHSYHWPCHRKHLTKTCLSWACHLLKGCFSCGCP

SEQ ID NO: 42 - protein, 14 aa, Homo sapiens
LRERAPRSCLPQLG

SEQ ID NO: 43 - protein, 10 aa, Homo sapiens
GWVYVASPIS

SEQ ID NO: 44 - protein, 6 aa, Homo sapiens
HAVESA

SEQ ID NO: 45 - protein, 4 aa, Homo sapiens
LSPS

SEQ ID NO: 46 - protein, 23 aa, Homo sapiens
PLGSQSSKQRDVAACGQHAQVPC

SEQ ID NO: 47 - protein, 22 aa, Homo sapiens
LSTYFCSFKKEFNVFGCIFLGG

SEQ ID NO: 48 - protein, 6 aa, Homo sapiens
GWAKTW

SEQ ID NO: 49 - DNA, 1287 bp, Homo sapiens
  misc_feature (11): A, T, C or G
aggggcccagnccccgccgtcgcgccagccccgcggccccagcgggccgggcactcgcaaccgcgctcgggcggcggacgctcgcgccggctcaccatgcactgccacgccgagctgaggctgagctcgcggccagctcaaagcagccaggcggcgctacaagactttcatgatcgacgagatcctctccaaggagacctgcgattactttgagaaactttccctctactccgtgtgcccgtcgctggtcgtgcgacccaagcccctgcattcctgtacgggctccccttccctgcgggcatatccgctcctctcggtgatcacccgccagcccactgtcatctcccacctggtccctgccaccccgggaatcgcccaggcactgtcctgccaccaggtcaccgaggcggtctctgctgaggccccagggggcgaggccctagccagcagcgagtcagagacggaacagcccacgccccgacagaagaagccccgccggagtcgcaccatcttcaccgagctgcagctcatgggcctggagaagaaattccagaagcagaagtatttgtcaaccccagacaggttggacttggctcagtctctgggactcactcagctgcaggtgaagacctggtatcagaatcgcaggatgaaatggaagaaaatggttcttaaaggtggacaggaagcacccacaaaacccaaaggtcgccccaagaagaactccatccccacatcagaagagattgaagctgaagagaagatgaacagccaggcccagggtcaggagcagctggagccctctcaggggcaggaggagctctgtgaagcacaggaaccgaaagcacgtgatgtccccttagagatggcagagccaccagacccgccccaggagttgccaataccctcttcggaacccccaccattaagctaaagtaaaacccttttgagggaagagggagactggggagaagggaaaagagagaaggcagggagagtagggagagaaaaccttccagcagcccagtaaactgcgggcgaagagatctacccgtctccctccctcccacagttaccattgagcctgtcatcgcaagcatttgacaaagacttgcttgtcttgggcctgtcacctcctgaaaggctgctttagctgtggatgcccttgattaagggagagagcgcctaggagctgcctgccccagctggggtgacggctgtagggctgggtctatgttgcaagccctatatcctagcatgcagtggaaagtgcttwgcghwattccg

SEQ ID NO: 50 - DNA, 244 bp, Homo sapiens
tcaccatgcactgccacgccgagctgaggctgagctcgcggccagctcaaagcagccaggcggcgctacaagactttcatgatcgacgagatcctctccaaggagacctgcgattactttgagaaactttccctctactccgtgtgcccgtcgctggtcgtgcgacccaagcccctgcattcctgtacgggtaagacgctccgctaggggataagtggggttcggtagctttcacgtccgtgta

SEQ ID NO: 51 - DNA, 435 bp, Homo sapiens
caggtcctggcctgcttccccacaccgttccctgtggtgacctgcctccctgcttgttttccaggctccccttccctgcgggcatatccgctcctctcggtgatcacccgccagcccactgtcatctcccacctggtccctgccaccccgggaatcgcccaggcactgtcctgccaccaggtcaccgaggcggtctctgctgaggccccagggggcgaggccctagccagcagcgagtcagagacggaacagcccacgccccgacagaagaagccccgccggagtcgcaccatcttcaccgagctgcagctcatgggcctggagaagaaattccagaagcagaagtatttgtcaaccccagacaggtgaggacgcagggaagggactctccgcagtgaaggcccctgggaacgggagacttgctcccattgtggg

SEQ ID NO: 52 - DNA, 285 bp, Homo sapiens
tcctgctgcctcccattctgctcacctttcctttgtatcttgtgccttctagaggttggacttggctcagtctctgggactcactcagctgcaggtgaagacctggtatcagaatcgcaggatgaaatggaagaaaatggtaagaaaggagtgactaaccatgatcccttcctgatgggaaggacttttactccagggctgttgggagggaggccggaccatttggcagtttgggctgcagagattggaaggcttctatgctgctggcttgcggggaagctgttg

SEQ ID NO: 53 - DNA, 357 bp, Homo sapiens
tggaaaggtggagggaaggaattatttcttagattcaataacaattttttctcacgtaggttcttaaaggtggacaggaagcacccacaaaacccaaaggtcgccccaagaagaactccatccccacatcagaagagattgaagctgaagagaagatgaacagccaggcccagggtcaggagcagctggagccctctcaggggcaggaggagctctgtgaagcacaggaaccgaaagcacgtgatgtccccttagagatggcagagccaccagacccgccccaggagttgccaataccctcttcggaacccccaccattaagctaaagtaaaacccttttgagggaagagggagact

SEQ ID NO: 54 - protein, 254 aa, Homo sapiens
MIDEILSKETCDYFEKLSLYSVCPSLVVRPKPLHSCTGSPSLRAYPLLSVITRQPTVISHLVPATPGIAQALSCHQVTEAVSAEAPGGEALASSESETEQPTPRQKKPRRSRTIFTELQLMGLEKKFQKQKYLSTPDRLDLAQSLGLTQLQVKTWYQNRRMKWKKMVLKGGQEAPTKPKGRPKKNSIPTSEEIEAEEKMNSQAQGQEQLEPSQGQEELCEAQEPKARDVPLEMAEPPDPPQELPIPSSEPPPLS